(19) United States
(12) Patent Application Publication
(10) Pub. No.: US 2011/0136792 A1
(43) Pub. Date: Jun. 9, 2011

(54) NOVEL CARBOXYLIC ACID ANALOGS AS GLYCOGEN SYNTHASE ACTIVATORS

(76) Inventors: David Robert Bolin, Montclair, NJ (US); Stuart Hayden, Manalapan, NJ (US); Yimin Qian, Wayne, NJ (US); Kshitij Chhabilbhai Thakkar, Clifton, NJ (US)

(21) Appl. No.: 12/908,199

(22) Filed: Oct. 20, 2010

Related U.S. Application Data
(60) Provisional application No. 61/266,680, filed on Dec. 4, 2009.

(51) Int. Cl. A61K 31/198; C07C 233/48; C07D 295/185; C07C 271/22; C07C 311/19; C07D 233/90; C07D 213/82; C07D 213/81; C07D 307/24; C07D 279/02; A61K 31/5377; A61K 31/27; A61K 31/4164; A61K 31/455; A61K 31/4458; A61K 31/341; A61P 3/00; A61K 31/54; A61P 3/10

(52) U.S. Cl. 514/222.2; 562/444; 544/169; 560/27; 562/430; 548/333.5; 546/316; 546/323; 549/487; 544/3; 514/563; 514/237.5; 514/486; 514/562; 514/400; 514/355; 514/354; 514/471

(57) ABSTRACT

Provided herein are compounds of the formula (I):

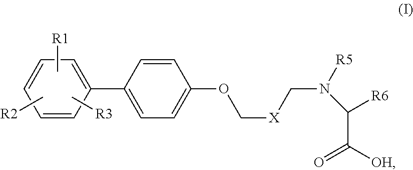

as well as pharmaceutically acceptable salts thereof, wherein the substituents are as those disclosed in the specification. These compounds, and the pharmaceutical compositions containing them, are useful for the treatment of metabolic diseases and disorders such as, for example, type II diabetes mellitus.

### PRIORITY TO RELATED APPLICATION(S)

This application claims the benefit of U.S. Provisional Application No. 61/266,680, filed Dec. 4, 2009, which is hereby incorporated by reference in its entirety.

### FIELD OF THE INVENTION

The invention is directed to compounds, salts and pharmaceutical compositions useful as activators of glycogen synthase for the treatment of metabolic diseases and disorders.

All documents cited or relied upon below are expressly incorporated herein by reference.

### BACKGROUND OF THE INVENTION

Diabetes mellitus is a common and serious disorder, affecting 10 million people in the U.S. [Harris, M. I. Diabetes Care 1998 21 (3S) Supplement, 11C], putting them at increased risk of stroke, heart disease, kidney damage, blindness, and amputation. Diabetes is characterized by decreased insulin secretion and/or an impaired ability of peripheral tissues to respond to insulin, resulting in increased plasma glucose levels. The incidence of diabetes is increasing, and the increase has been associated with increasing obesity and a sedentary life. There are two forms of diabetes: insulin-dependent and non-insulin-dependent, with the great majority of diabetics suffering from the non-insulin-dependent form of the disease, known as type 2 diabetes or non-insulin-dependent diabetes mellitus (NIDDM). Because of the serious consequences, there is an urgent need to control diabetes.

Treatment of NIDDM generally starts with weight loss, a healthy diet and an exercise program. However, these factors are often unable to control the disease, and there are a number of drug treatments available, including insulin, metformin, sulfonylureas, acarbose, and thiazolidinediones. Each of these treatments has disadvantages and there is an ongoing need for new drugs to treat diabetes.

Metformin is an effective agent that reduces fasting plasma glucose levels and enhances the insulin sensitivity of peripheral tissue, mainly through an increase in glycogen synthesis [De Fronzo, R. A. Drugs 1999, 58 Suppl. 1, 29]. Metformin also leads to reductions in the levels of LDL cholesterol and triglycerides [Inzucchi, S. E. JAMA 2002, 287, 360]. However, it loses its effectiveness over a period of years [Turner, R. C. et al. JAMA 1999, 281, 2005].

Thiazolidinediones are activators of the nuclear receptor peroxisome-proliferator activated receptor-gamma. They are effective in reducing blood glucose levels, and their efficacy has been attributed primarily to decreasing insulin resistance in skeletal muscle [Tadayyon, M. and Smith, S. A. Expert Opin. Investig. Drugs 2003, 12, 307]. One disadvantage associated with the use of thiazolidinediones is weight gain.

Sulfonylureas bind to the sulfonylurea receptor on pancreatic beta cells, stimulate insulin secretion, and consequently reduce blood glucose levels. Weight gain is also associated with the use of sulfonylureas [Inzucchi, S. E. JAMA 2002, 287, 360] and, like metformin, they lose efficacy over time [Turner, R. C. et al. JAMA 1999, 281, 2005]. A further problem often encountered in patients treated with sulfonylureas is hypoglycemia [Salas, M. and Caro, J. J. Adv. Drug React. Tox. Rev. 2002, 21, 205-217].

Acarbose is an inhibitor of the enzyme alpha-glucosidase, which breaks down disaccharides and complex carbohydrates in the intestine. It has lower efficacy than metformin or the sulfonylureas, and it causes intestinal discomfort and diarrhea which often lead to the discontinuation of its use [Inzucchi, S. E. JAMA 2002, 287, 360].

Because none of these treatments is effective over the long term without serious side effects, there is a need for new drugs for the treatment of type 2 diabetes.

In skeletal muscle and liver, there are two major pathways of glucose utilization: glycolysis, or oxidative metabolism, where glucose is oxidized to pyruvate; and glycogenesis, or glucose storage, where glucose is stored in the polymeric form glycogen. The key step in the synthesis of glycogen is the addition of the glucose derivative UDP-glucose to the growing glycogen chain, and this step is catalyzed by the enzyme glycogen synthase [Cid, E. et al. J. Biol. Chem. 2000, 275, 33614]. There are two isoforms of glycogen synthase, found in liver [Bai, G. et al. J. Biol. Chem. 1990, 265, 7843] and in other peripheral tissues including muscle [Browner, M. F. et al. Proc. Nat. Acad. Sci. U.S.A. 1989, 86, 1443]. There is clinical and genetic evidence implicating both forms of glycogen synthase in metabolic diseases such as type 2 diabetes and cardiovascular disease. Both basal and insulin-stimulated glycogen synthase activity in muscle cells from diabetic subjects were significantly lower than in cells from lean non-diabetic subjects [Henry, R. R. et al. J. Clin. Invest. 1996, 98, 1231-1236; Nikoulina, S. E. et al. J. Clin. Enocrinol. Metab. 2001, 86, 4307-4314]. Furthermore, several studies have shown that levels of muscle [Eriksson, J. et al. N. Engl. J. Mod. 1989, 331, 337; Schulman, R. G. et al. N. Engl. J. Med. 1990, 332, 223; Thorburn, A. W. et al. J. Clin. Invest. 1991, 87, 489] and liver [Krssak, M. et. al. Diabetes 2004, 53, 3048] glycogen are lower in diabetic patients than in control subjects. In addition, genetic studies have shown associations in several populations between type 2 diabetes and/or cardiovascular disease and mutation/deletion in the GYS1 gene encoding the muscle isoform of glycogen synthase [Orhu-Melander, M. et al. Diabetes 1999, 48, 918; Fredriksson, J. et. al. PLoS ONE 2007, 3, e285; Kolhberg G. et. al. N. Engl. J. Med. 2007, 357, 1507]. Patients lacking GYS2 encoding the liver isoform of glycogen synthase, suffer from fasting ketotic hypoglycemia and postprandial hyperglycemia, hyperlactanemia and hyperlipidemia, supporting the essential role of liver GS in maintaining normal nutrient metabolism. [Weinstein, D. A. et. al. Mol. Genetics and Metabolism, 2006, 87, 284]

Glycogen synthase is subject to complex regulation, involving phosphorylation in at least nine sites [Lawrence, J. C., Jr. and Roach, P. J. Diabetes 1997, 46, 541]. The dephosphorylated form of the enzyme is active. Glycogen synthase is phosphorylated by a number of enzymes of which glycogen synthase kinase 3β (GSK3β) is the best understood [Tadayyon, M. and Smith, S. A. Expert Opin. Investig. Drugs 2003, 12, 307], and glycogen synthase is dephosphorylated by protein phosphatase type I (PP1) and protein phosphatase type 2A (PP2A). In addition, glycogen synthase is regulated by an endogenous ligand, glucose-6-phosphate which allosterically stimulates the activity of glycogen synthase by causing a change in the conformation of the enzyme that renders it more susceptible to dephosphorylation by the protein phosphatases to the active form of the enzyme [Gomis, R. R. et al. J. Biol. Chem. 2002, 277, 23246].

Several mechanisms have been proposed for the effect of insulin in reducing blood glucose levels, each resulting in an increase in the storage of glucose as glycogen. First, glucose uptake is increased through recruitment of the glucose transporter GLUT4 to the plasma membrane [Holman, G. D. and Kasuga, M. Diabetologia 1997, 40, 991]. Second, there is an increase in the concentration of glucose-6-phosphate, the allosteric activator of glycogen synthase [Villar-Palasi, C. and Guinovart, J. J. FASEB J. 1997, 11, 544]. Third, a kinase cascade beginning with the tyrosine kinase activity of the insulin receptor results in the phosphorylation and inactivation of GSK3β, thereby preventing the deactivation of glycogen synthase [Cohen, P. Biochem. Soc. Trans. 1993, 21, 555; Yeaman, S. J. Biochem. Soc. Trans. 2001, 29, 537].

Because a significant decrease in the activity of glycogen synthase has been found in diabetic patients, and because of its key role in glucose utilization, the activation of the enzyme glycogen synthase holds therapeutic promise for the treatment of metabolic diseases such as type 2 diabetes and cardiovascular diseases. The only known allosteric activators of the enzyme are glucose-6-phosphate [Leloir, L. F. et al. Arch. Biochem. Biophys. 1959, 81, 508] and glucosamine-6-phosphate [Virkamaki, A. and Yki-Jarvinen, H. Diabetes 1999, 48, 1101].

The following biaryloxymethylarenecarboxylic acids are reported to be commercially available from Otava, Toronto, Canada, Akos Consulting & Solutions, Steinen, Germany or Princeton BioMolecular Research, Monmouth Junction, N.J.: 4-(biphenyl-4-yloxymethyl)-benzoic acid, 3-(biphenyl-4-yloxymethyl)-benzoic acid, [4-(biphenyl-4-yloxymethyl)-phenyl]-acetic acid, [4-(4′-methyl-biphenyl-4-yloxymethyl)-phenyl]-acetic acid, 4-(4′-methyl-biphenyl-4-yloxymethyl)-benzoic acid, 3-(3-bromo-biphenyl-4-yloxymethyl)-benzoic acid, [4-(3-bromo-biphenyl-4-yloxymethyl)-phenyl]-acetic acid, 2-(4′-methyl-biphenyl-4-yloxymethyl)-benzoic acid, 5-(biphenyl-4-yloxymethyl)-furan-2-carboxylic acid, 5-(4′-methyl-biphenyl-4-yloxymethyl)-furan-2-carboxylic acid, 5-(3-bromo-biphenyl-4-yloxymethyl)-furan-2-carboxylic acid, 4-(biphenyl-4-yloxymethyl)-5-methyl-furan-2-carboxylic acid, 5-methyl-4-(4′-methyl-biphenyl-4-yloxymethyl)-furan-2-carboxylic acid, 4-(3-bromo-biphenyl-4-yloxymethyl)-5-methyl-furan-2-carboxylic acid, 2-(biphenyl-4-yloxymethyl)-4-methyl-thiazole-5-carboxylic acid, [2-(biphenyl-4-yloxymethyl)-thiazol-4-yl]-acetic acid, [2-(4′-methyl-biphenyl-4-yloxymethyl)-thiazol-4-yl]-acetic acid and [5-(biphenyl-4-yloxymethyl)-[1,3,4]oxadiazol-2-yl]-acetic acid.

Some biaryloxymethylarenecarboxylic acids are known in the art. However, none of these known compounds have been associated with either the treatment of diseases mediated by the activation of the glycogen synthase enzyme or to any pharmaceutical composition for the treatment of diseases mediated by the activation of the glycogen synthase enzyme. Andersen, H. S. et al. WO 9740017 discloses the structure and synthetic route to 3-(biphenyl-4-yloxymethyl)-benzoic acid as an intermediate in the synthesis of SH2 inhibitors. Winkelmann, E. et al. DE 2842243 discloses 5-(biphenyl-4-yloxymethyl)-thiophene-2-carboxylic acid as a hypolipemic agent. Mueller, T. et al. DE 4142514 discloses 2-(biphenyl-3-yloxymethyl)-benzoic acid as a fungicide. Ghosh, S. S. et al. WO 2004058679 discloses biaryloxymethylarene acids as ligands of adenine nucleoside translocase. Van Zandt, M. C. WO 2008033455 discloses biphenyl and heteroarylphenyl derivatives as protein phosphatase-1B inhibitors.

Glycogen synthase activators and stimulators of glycogen production have been reported. Chu, C. A et al. US 20040266856 discloses biaryoxymethylarenecarboxylic acids as glycogen synthase activators. Chu, C.A. WO 2005000781 discloses biaryloxymethylarene carboxylic acids as activators of glycogen synthase. Yang, S—P. and Huang, Y. US 20050095219 discloses hyaluronic acid compounds that stimulate glycogen production. Gillespie, P. et al. WO 2005075468 discloses biaryoxymethylarene carboxylic acids as glycogen synthase activators. Gillespie, P. et al. WO 2006058648 discloses biaryoxymethylarene carboxylic acids as glycogen synthase activators. Bucala, R. et al. WO 2007044622 discloses macrophage migration inhibitory factor agonists that stimulate glycogen production.

### SUMMARY OF THE INVENTION

The present invention is directed to compounds of the formula I:

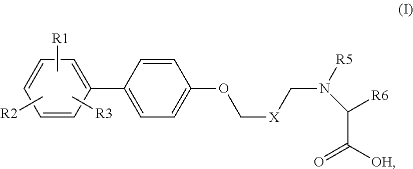

as well as pharmaceutically acceptable salts thereof, pharmaceutical compositions containing them and to methods of treating diseases and disorders. The compounds and compositions disclosed herein are glycogen synthase activators and are useful for the treatment of metabolic diseases and disorders, preferably diabetes mellitus, more preferably type II diabetes mellitus.

### DETAILED DESCRIPTION OF THE INVENTION

In an embodiment of the present invention, provided is a compound of Formula (I):

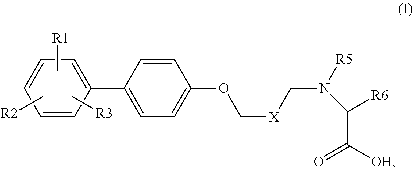

wherein:
R1, R2, R3, independently of each other, is hydrogen, halogen, lower alkyl or alkoxy;
X is pyridine, thiazole, unsubstituted phenyl or phenyl substituted with R4;
R4 is halogen;
R5 is hydrogen, an acyl moiety, —SO₂-lower alkyl, —SO₂-aryl, —SO₂-cycloalkyl, or unsubstituted lower alkyl or lower alkyl substituted with phenyl;
R6 is hydrogen or lower alkyl; or
R5 and R6, together with the nitrogen atom to which they are attached, form a 5- or 6-membered heterocyclic ring, optionally containing a further heteroatom selected from oxygen or sulfur, said heterocyclic ring being unsubstituted or mono- or bi-substituted with (═O),
or a pharmaceutically acceptable salt thereof.

Preferably, X is unsubstituted phenyl or phenyl substituted with R4; R5 is hydrogen, an acyl moiety, —SO₂-lower alkyl, —SO₂-aryl, —SO₂-cycloalkyl, unsubstituted lower alkyl or lower alkyl substituted with phenyl; and R6 is hydrogen.

Preferably, X is unsubstituted phenyl or phenyl substituted with R4; and R5 and R6, together with the nitrogen atom to which they are attached, form a 5- or 6-membered heterocyclic ring, optionally containing a further heteroatom selected from oxygen or sulfur, said heterocyclic ring being unsubstituted or mono- or bi-substituted with (═O).

Preferably, X is thiazole or pyridine; R5 is hydrogen, an acyl moiety, —SO₂-lower alkyl, —SO₂-aryl, —SO₂-cycloalkyl, unsubstituted lower alkyl or lower alkyl substituted with phenyl; and R6 is hydrogen.

Preferably, X is thiazole or pyridine; and R5 and R6, together with the nitrogen atom to which they are attached, form a 5- or 6-membered heterocyclic ring, optionally containing a further heteroatom selected from oxygen or sulfur, said heterocyclic ring being unsubstituted or mono- or bi-substituted with (═O).

Preferably, R1, R2, R3, independently of each other, is hydrogen, fluoro, chloro, methyl or methoxy.

Preferably, R1 is hydrogen or fluoro.

Preferably, R2 is fluoro.

Preferably, R3 is fluoro, chloro or methoxy.

Preferably, X is unsubstituted phenyl.

Preferably, X is thiazole.

Preferably, X is pyridine.

Preferably, R4 is fluorine.

Preferably, R5 is an acyl moiety.

Preferably, R5 is an acyl moiety selected from the group consisting of: —C(O)-lower alkyl, branched or unbranched, unsubstituted or substituted with alkoxy or cycloalkyl, —C(O)-cycloalkyl, —C(O)-heterocycloalkyl, unsubstituted or substituted with methyl, —C(O)-aryl, —C(O)-alkoxy, and —C(O)-heteroaryl, unsubstituted or substituted with methyl.

Preferably, R5 is an acyl moiety selected from the group consisting of: —C(O)C(CH₃)₃, —C(O)CH₂CH(CH₃)₂, —C(O)-morpholine, —C(O)-cyclobutane, —C(O)-phenyl, —C(O)OCH(CH₃)₂, —C(O)-methylimidazole, —C(O)-pyridine, —C(O)C(CH₃)CH₂OCH₃, —C(O)OCH₃, —C(O)OCH₂CH₃, —C(O)CH₃, —C(O)-cyclopropane, —C(O)CH₂CH₃, —C(O)CH₂-cyclopropane, —C(O)-tetrahydrofuran, —C(O)CH₂CH₂OCH₃, —C(O)-thiazole, —C(O)CH₂OCH₃ and —C(O)-methylpiperidine.

Preferably, R5 is hydrogen, —SO₂-lower alkyl, —SO₂-aryl, —SO₂-cycloalkyl, unsubstituted lower alkyl or lower alkyl substituted with phenyl.

Preferably, R5 is hydrogen, —SO₂CH₂CH₃, —SO₂-phenyl, —SO₂-cyclopentane, —SO₂CH₃, —CH₂-phenyl or —CH₂CH₃.

Preferably, R5 and R6, together with the nitrogen atom to which they are attached, form a 5- or 6-membered heterocyclic ring, optionally containing a further heteroatom selected from oxygen or sulfur, said heterocyclic ring being unsubstituted or mono- or bi-substituted with (═O).

Preferably, said 5- or 6-membered heterocyclic ring is dioxo-isothiazolidine, oxo-pyrrolidine, pyrrolidine or dioxo-thiazinane.

Preferably, R6 is hydrogen.

Preferably, said compound of formula (I) is:
[[3-(4′,5′-Difluoro-2′-methoxy-biphenyl-4-yloxymethyl)-benzyl]-(2,2-dimethyl-propionyl)-amino]-acetic acid;
[[3-(4′,5′-Difluoro-2′-methoxy-biphenyl-4-yloxymethyl)-benzyl]-(3-methyl-butyryl)-amino]-acetic acid;
[[3-(4′,5′-Difluoro-2′-methoxy-biphenyl-4-yloxymethyl)-benzyl]-(morpholine-4-carbonyl)-amino]-acetic acid;
{Cyclobutanecarbonyl-[3-(4′,5′-difluoro-2′-methoxy-biphenyl-4-yloxymethyl)-benzyl]-amino}-acetic acid;
{Benzoyl-[3-(4′,5′-difluoro-2′-methoxy-biphenyl-4-yloxymethyl)-benzyl]-amino}-acetic acid;
{[3-(4′,5′-Difluoro-2′-methoxy-biphenyl-4-yloxymethyl)-benzyl]-isopropoxycarbonyl-amino}-acetic acid;
{[3-(4′,5′-Difluoro-2′-methoxy-biphenyl-4-yloxymethyl)-benzyl]-ethanesulfonyl-amino}-acetic acid;
{Benzenesulfonyl-[3-(4′,5′-difluoro-2′-methoxy-biphenyl-4-yloxymethyl)-benzyl]-amino}-acetic acid;
{Cyclopropanesulfonyl-[3-(4′,5′-difluoro-2′-methoxy-biphenyl-4-yloxymethyl)-benzyl]-amino}-acetic acid;
{[3-(4′,5′-Difluoro-2′-methoxy-biphenyl-4-yloxymethyl)-benzyl]-methanesulfonyl-amino}-acetic acid;
[[3-(2′,4′-Difluoro-biphenyl-4-yloxymethyl)-benzyl]-(2,2-dimethyl-propionyl)-amino]-acetic acid;
{Cyclopropanecarbonyl-[3-(2′,4′-difluoro-biphenyl-4-yloxymethyl)-benzyl]-amino}-acetic acid;
[[3-(2′,4′-Difluoro-biphenyl-4-yloxymethyl)-benzyl]-(morpholine-4-carbonyl)-amino]-acetic acid;
[[3-(2′,4′-Difluoro-biphenyl-4-yloxymethyl)-benzyl]-(1-methyl-imidazole-2-carbonyl)-amino]-acetic acid;
[[3-(2′,4′-Difluoro-biphenyl-4-yloxymethyl)-benzyl]-(pyridine-3-carbonyl)-amino]-acetic acid;
[[3-(2′,4′-Difluoro-biphenyl-4-yloxymethyl)-benzyl]-(pyridine-2-carbonyl)-amino]-acetic acid;
[[3-(2′,4′-Difluoro-biphenyl-4-yloxymethyl)-benzyl]-(3-methoxy-2-methyl-propionyl)-amino]-acetic acid;
{Ethanesulfonyl-[3-(2′,4′,5′-trifluoro-biphenyl-4-yloxymethyl)-benzyl]-amino}-acetic acid;
{[3-(4′,5′-Difluoro-2′-methoxy-biphenyl-4-yloxymethyl)-benzyl]-methoxycarbonyl-amino}-acetic acid;
{[3-(4′,5′-Difluoro-2′-methoxy-biphenyl-4-yloxymethyl)-benzyl]-ethoxycarbonyl-amino}-acetic acid;
{Acetyl-[3-(4′,5′-difluoro-2′-methoxy-biphenyl-4-yloxymethyl)-benzyl]-amino}-acetic acid;
{Cyclopropanecarbonyl-[3-(4′,5′-difluoro-2′-methoxy-biphenyl-4-yloxymethyl)-benzyl]-amino}-acetic acid;
{[3-(4′,5′-Difluoro-2′-methoxy-biphenyl-4-yloxymethyl)-benzyl]-propionyl-amino}-acetic acid;
{(2-Cyclopropyl-acetyl)-[3-(4′,5′-difluoro-2′-methoxy-biphenyl-4-yloxymethyl)-benzyl]-amino}-acetic acid;
[[3-(4′,5′-Difluoro-2′-methoxy-biphenyl-4-yloxymethyl)-benzyl]-((S)-tetrahydro-furan-2-carbonyl)-amino]-acetic acid;
[[3-(4′,5′-Difluoro-2′-methoxy-biphenyl-4-yloxymethyl)-benzyl]-(3-methoxy-propionyl)-amino]-acetic acid;
[[3-(4′,5′-Difluoro-2′-methoxy-biphenyl-4-yloxymethyl)-benzyl]-(thiazole-4-carbonyl)-amino]-acetic acid;
[[3-(4′,5′-Difluoro-2′-methoxy-biphenyl-4-yloxymethyl)-benzyl]-(2-methoxy-acetyl)-amino]-acetic acid;
[[3-(4′,5′-Difluoro-2′-methoxy-biphenyl-4-yloxymethyl)-benzyl]-(1-methyl-1H-imidazole-4-carbonyl)-amino]-acetic acid;
[[3-(4′,5′-difluoro-2′-methoxy-biphenyl-4-yloxymethyl)-benzyl]-(pyridine-3-carbonyl)-amino]-acetic acid;
[[3-(4′,5′-Difluoro-2′-methoxy-biphenyl-4-yloxymethyl)-benzyl]-(3-methoxy-2-methyl-propionyl)-amino]-acetic acid;
[[3-(4′,5′-Difluoro-2′-methoxy-biphenyl-4-yloxymethyl)-benzyl]-(1-methyl-piperidine-4-carbonyl)-amino]-acetic acid;
[[3-(4′,5′-Difluoro-2′-methoxy-biphenyl-4-yloxymethyl)-benzyl]-((R)-tetrahydro-furan-2-carbonyl)-amino]-acetic acid;
[[3-(4′,5′-Difluoro-2′-methoxy-biphenyl-4-yloxymethyl)-benzyl]-(tetrahydro-furan-3-carbonyl)-amino]-acetic acid;
{Benzyl-[3-(4′,5′-difluoro-2′-methoxy-biphenyl-4-yloxymethyl)-benzyl]-amino}-acetic acid;
{[3-(4′,5′-Difluoro-2′-methoxy-biphenyl-4-yloxymethyl)-benzyl]-ethyl-amino}-acetic acid;
[3-(4′,5′-Difluoro-2′-methoxy-biphenyl-4-yloxymethyl)-benzylamino]-acetic acid;
1,1-Dioxo-2-[3-(2′,4′,5′-trifluoro-biphenyl-4-yloxymethyl)-benzyl]-1lambda*6*-isothiazolidine-3-carboxylic acid;
2-[3-(4′,5′-Difluoro-2′-methyl-biphenyl-4-yloxymethyl)-benzyl]-1,1-dioxo-1lambda*6*-isothiazolidine-3-carboxylic acid;
2-[3-(2′-Chloro-4′,5′-difluoro-biphenyl-4-yloxymethyl)-benzyl]-1,1-dioxo-1lambda*6*-isothiazolidine-3-carboxylic acid;
2-[3-(2′,4′-Difluoro-biphenyl-4-yloxymethyl)-4-fluoro-benzyl]-1,1-dioxo-1lambda*6*-isothiazolidine-3-carboxylic acid;
2-[5-(2′,4′-Difluoro-biphenyl-4-yloxymethyl)-2-fluoro-benzyl]-1,1-dioxo-1lambda*6*-isothiazolidine-3-carboxylic acid;
2-[3-(4′,5′-Difluoro-2′-methoxy-biphenyl-4-yloxymethyl)-benzyl]-1,1-dioxo-1lambda*6*-isothiazolidine-3-carboxylic acid;
(R)-2-[3-(4′,5′-Difluoro-2′-methoxy-biphenyl-4-yloxymethyl)-benzyl]-1,1-dioxo-1lambda*6*-isothiazolidine-3-carboxylic acid;
(S)-2-[3-(4′,5′-Difluoro-2′-methoxy-biphenyl-4-yloxymethyl)-benzyl]-1,1-dioxo-1lambda*6*-isothiazolidine-3-carboxylic acid;
(S)-1,1-Dioxo-2-[3-(2′,4′,5′-trifluoro-biphenyl-4-yloxymethyl)-benzyl]-1lambda*6*-isothiazolidine-3-carboxylic acid;
(R)-1,1-Dioxo-2-[3-(2′,4′,5′-trifluoro-biphenyl-4-yloxymethyl)-benzyl]-1lambda*6*-isothiazolidine-3-carboxylic acid;
(S)-2-[3-(4′,5′-Difluoro-2′-methyl-biphenyl-4-yloxymethyl)-benzyl]-1,1-dioxo-1lambda*6*-isothiazolidine-3-carboxylic acid;
(R)-2-[3-(4′,5′-Difluoro-2′-methyl-biphenyl-4-yloxymethyl)-benzyl]-1,1-dioxo-1lambda*6*-isothiazolidine-3-carboxylic acid;
(R)-2-[3-(2′-Chloro-4′,5′-difluoro-biphenyl-4-yloxymethyl)-benzyl]-1,1-dioxo-1lambda*6*-isothiazolidine-3-carboxylic acid;
(S)-2-[3-(2′-Chloro-4′,5′-difluoro-biphenyl-4-yloxymethyl)-benzyl]-1,1-dioxo-1lambda*6*-isothiazolidine-3-carboxylic acid;
(S)-1-[3-(4′,5′-Difluoro-2′-methoxy-biphenyl-4-yloxymethyl)-benzyl]-5-oxo-pyrrolidine-2-carboxylic acid;
(S)-5-Oxo-1-[3-(2′,4′,5′-trifluoro-biphenyl-4-yloxymethyl)-benzyl]-pyrrolidine-2-carboxylic acid;
(S)-1-[3-(2′,4′-Difluoro-biphenyl-4-yloxymethyl)-benzyl]-pyrrolidine-2-carboxylic acid trifluoro-acetic acid salt;
(R)-2-[3-(4′,5′-Difluoro-2′-methoxy-biphenyl-4-yloxymethyl)-benzyl]-1,1-dioxo-1lambda*6*-[1,2]thiazinane-3-carboxylic acid;
(S)-2-[3-(4′,5′-Difluoro-2′-methoxy-biphenyl-4-yloxymethyl)-benzyl]-1,1-dioxo-1lambda*6*-[1,2]thiazinane-3-carboxylic acid;
(R)-1,1-Dioxo-2-[3-(2′,4′,5′-trifluoro-biphenyl-4-yloxymethyl)-benzyl]-1lambda*6*-[1,2]thiazinane-3-carboxylic acid;
(S)-1,1-Dioxo-2-[3-(2′,4′,5′-trifluoro-biphenyl-4-yloxymethyl)-benzyl]-1lambda*6*-[1,2]thiazinane-3-carboxylic acid;
(R)-1,1-Dioxo-2-[2-(2′,4′,5′-trifluoro-biphenyl-4-yloxymethyl)-thiazol-4-ylmethyl]-1lambda*6*-isothiazolidine-3-carboxylic acid;
(S)-1,1-Dioxo-2-[2-(2′,4′,5′-trifluoro-biphenyl-4-yloxymethyl)-thiazol-4-ylmethyl]-1lambda*6*-isothiazolidine-3-carboxylic acid;
(R)-2-[2-(4′,5′-Difluoro-2′-methoxy-biphenyl-4-yloxymethyl)-thiazol-4-ylmethyl]-1,1-dioxo-1lambda*6*-isothiazolidine-3-carboxylic acid;
(S)-2-[2-(4′,5′-Difluoro-2′-methoxy-biphenyl-4-yloxymethyl)-thiazol-4-ylmethyl]-1,1-dioxo-1lambda*6*-isothiazolidine-3-carboxylic acid;
2-[5-(4′,5′-Difluoro-2′-methoxy-biphenyl-4-yloxymethyl)-pyridin-3-ylmethyl]-1,1-dioxo-1lambda*6*-isothiazolidine-3-carboxylic acid; or
2-[5-(4′,5′-Difluoro-2′-methoxy-biphenyl-4-yloxymethyl)-pyridin-3-ylmethyl]-1,1-dioxo-1lambda*6*-isothiazolidine-3-carboxylic acid.

In another preferred embodiment, provided is a pharmaceutical composition, comprising a therapeutically effective amount of a compound according to formula (I) and a pharmaceutically acceptable carrier and/or adjuvant.

It is to be understood that the terminology employed herein is for the purpose of describing particular embodiments, and is not intended to be limiting. Further, although any methods, devices and materials similar or equivalent to those described herein can be used in the practice or testing of the invention, the preferred methods, devices and materials are now described.

As used herein, the term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “cycloalkyl” refers to a monovalent mono- or polycarbocyclic radical of three to ten, preferably three to six carbon atoms. This term is further exemplified by radicals such as cyclopropyl, cyclobutyl, cyclopentyl, cyclohexyl, cycloheptyl, bornyl, adamantyl, indenyl and the like. In a preferred embodiment, the “cycloalkyl” moieties can optionally be substituted with one, two, three or four substituents with the understanding that said substituents are not, in turn, substituted further unless indicated otherwise in the Examples or claims below. Examples of cycloalkyl moieties include, but are not limited to, optionally substituted cyclopropyl, optionally substituted cyclobutyl, optionally substituted cyclopentyl, optionally substituted cyclopentenyl, optionally substituted cyclohexyl, optionally substituted cyclohexylene, optionally substituted cycloheptyl.

The term “heterocycloalkyl” or “heterocyclic” denotes a mono- or polycyclic alkyl ring, wherein one, two or three of the carbon ring atoms is replaced by a heteroatom such as N, O or S. Examples of heterocycloalkyl groups include, but are not limited to, pyranyl, morpholinyl, thiomorpholinyl, piperazinyl, piperidinyl, pyrrolidinyl, tetrahydropyranyl, tetrahydrofuranyl, 1,3-dioxanyl, dioxidoisothiazolidine and the like. The heterocycloalkyl groups may be unsubstituted or substituted with one, two or three substituents and attachment may be through their carbon frame or through their heteroatom(s) where appropriate, with the understanding that said substituents are not, in turn, substituted further unless indicated otherwise in the Examples or claims below. An example of said substituent is (═O).

The term “lower alkyl”, alone or in combination with other groups, refers to a branched or straight-chain alkyl radical of one to nine carbon atoms, preferably one to six carbon atoms, most preferably one to four carbon atoms. This term is further exemplified by radicals such as methyl, ethyl, n-propyl, isopropyl, n-butyl, s-butyl, isobutyl, t-butyl, n-pentyl, 3-methylbutyl, n-hexyl, 2-ethylbutyl and the like.

As used herein, the term “acyl” means an optionally substituted alkyl, cycloalkyl, heterocyclic, aryl or heteroaryl group bound via a carbonyl group and includes groups such as acetyl, —C(O)-lower alkyl, branched or unbranched, unsubstituted or substituted with alkoxy or cycloalkyl, —C(O)-cycloalkyl, —C(O)-heterocycloalkyl, unsubstituted or substituted with methyl, —C(O)-aryl, —C(O)-alkoxy, and —C(O)-heteroaryl, unsubstituted or substituted with methyl, and the like.

The term “aryl” refers to an aromatic mono- or polycarbocyclic radical of 6 to 12 carbon atoms having at least one aromatic ring. Examples of such groups include, but are not limited to, phenyl and napthyl.

The alkyl, lower alkyl and aryl groups may be substituted or unsubstituted. When substituted, there will generally be, for example, 1 to 4 substituents present, with the understanding that said substituents are not, in turn, substituted further unless indicated otherwise in the Examples or claims below.

The term “heteroaryl,” refers to an aromatic mono- or polycyclic radical of 5 to 12 atoms having at least one aromatic ring containing one, two, or three ring heteroatoms selected from N, O, and S, with the remaining ring atoms being C. One or two ring carbon atoms of the heteroaryl group may be replaced with a carbonyl group. The heteroaryl group may be substituted independently with one, two, or three substituents, with the understanding that said substituents are not, in turn, substituted further unless indicated otherwise in the Examples or claims below. Examples of heteroaryl groups include, but are not limited to, pyridine, imidazole and thiazole.

As used herein, the term “alkoxy” means alkyl-O—; and “alkoyl” means alkyl-CO—. Alkoxy substituent groups or alkoxy-containing substituent groups may be substituted by, for example, one or more alkyl groups with the understanding that said substituents are not, in turn, substituted further unless indicated otherwise in the Examples or claims below.

As used herein, the term “halogen” means a fluorine, chlorine, bromine or iodine radical, preferably a fluorine, chlorine or bromine radical, and more preferably a fluorine or chlorine radical.

Compounds of formula (I) can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with chiral adsorbents or eluant). The invention embraces all of these forms.

As used herein, the term “pharmaceutically acceptable salt” means any pharmaceutically acceptable salt of the compound of formula (I). Salts may be prepared from pharmaceutically acceptable non-toxic acids and bases including inorganic and organic acids and bases. Such acids include, for example, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfuric, tartaric, oxalic, p-toluenesulfonic and the like. Particularly preferred are fumaric, hydrochloric, hydrobromic, phosphoric, succinic, sulfuric and methanesulfonic acids. Acceptable base salts include alkali metal (e.g. sodium, potassium), alkaline earth metal (e.g. calcium, magnesium) and aluminium salts.

In the practice of the method of the present invention, an effective amount of any one of the compounds of this invention or a combination of any of the compounds of this invention or a pharmaceutically acceptable salt thereof, is administered via any of the usual and acceptable methods known in the art, either singly or in combination. The compounds or compositions can thus be administered orally (e.g., buccal cavity), sublingually, parenterally (e.g., intramuscularly, intravenously, or subcutaneously), rectally (e.g., by suppositories or washings), transdermally (e.g., skin electroporation) or by inhalation (e.g., by aerosol), and in the form of solid, liquid or gaseous dosages, including tablets and suspensions. The administration can be conducted in a single unit dosage form with continuous therapy or in a single dose therapy ad libitum. The therapeutic composition can also be in the form of an oil emulsion or dispersion in conjunction with a lipophilic salt such as pamoic acid, or in the form of a biodegradable sustained-release composition for subcutaneous or intramuscular administration.

Useful pharmaceutical carriers for the preparation of the compositions hereof, can be solids, liquids or gases; thus, the compositions can take the form of tablets, pills, capsules, suppositories, powders, enterically coated or other protected formulations (e.g. binding on ion-exchange resins or packaging in lipid-protein vesicles), sustained release formulations, solutions, suspensions, elixirs, aerosols, and the like. The carrier can be selected from the various oils including those of petroleum, animal, vegetable or synthetic origin, e.g., peanut oil, soybean oil, mineral oil, sesame oil, and the like. Water, saline, aqueous dextrose, and glycols are preferred liquid carriers, particularly (when isotonic with the blood) for injectable solutions. For example, formulations for intravenous administration comprise sterile aqueous solutions of the active ingredient(s) which are prepared by dissolving solid active ingredient(s) in water to produce an aqueous solution, and rendering the solution sterile. Suitable pharmaceutical excipients include starch, cellulose, talc, glucose, lactose, gelatin, malt, rice, flour, chalk, silica, magnesium stearate, sodium stearate, glycerol monostearate, sodium chloride, dried skim milk, glycerol, propylene glycol, water, ethanol, and the like. The compositions may be subjected to conventional pharmaceutical additives such as preservatives, stabilizing agents, wetting or emulsifying agents, salts for adjusting osmotic pressure, buffers and the like. Suitable pharmaceutical carriers and their formulation are described in Remington's Pharmaceutical Sciences by E. W. Martin. Such compositions will, in any event, contain an effective amount of the active compound together with a suitable carrier so as to prepare the proper dosage form for proper administration to the recipient.

The dose of a compound of the present invention depends on a number of factors, such as, for example, the manner of administration, the age and the body weight of the subject, and the condition of the subject to be treated, and ultimately will be decided by the attending physician or veterinarian. Such an amount of the active compound as determined by the attending physician or veterinarian is referred to herein, and in the claims, as a “therapeutically effective amount”. For example, the dose of a compound of the present invention is typically in the range of about 1 to about 1000 mg per day. Preferably, the therapeutically effective amount is in an amount of from about 1 mg to about 500 mg per day.

It will be appreciated, that the compounds of general formula (I) in this invention may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

Chemicals may be purchased from companies such as for example Aldrich, Argonaut Technologies, VWR and Lancaster. Chromatography supplies and equipment may be purchased from such companies as for example AnaLogix, Inc, Burlington, Wis.; Biotage AB, Charlottesville, Va.; Analytical Sales and Services, Inc., Pompton Plains, N.J.; Teledyne Isco, Lincoln, Nebr.; VWR International, Bridgeport, N.J.; Varian Inc., Palo Alto, Calif., and Multigram II Mettler Toledo Instrument Newark, Del. Biotage, ISCO and Analogix columns are pre-packed silica gel columns used in standard chromatography.

Definitions as used herein include:

GS is glycogen synthase,
THF is tetrahydrofuran,

### DMF is N,N-dimethylformamide,

DMSO is dimethylsulfoxide,
DCM is dichloromethane,
MeOH is methanol,
EtOH is ethanol,
EtOAc is ethyl acetate,
LAH is lithium aluminum hydride,

### NBS is N-bromosuccinimide,

DCC is N,N′-dicyclohexylcarbodiimide,
BOP reagent is (benzotriazole-1-yloxy)-tris(dimethylamino)phosphonium hexafluorophosphate,
Pd(dppf)Cl₂ is [1,1′-bis(diphenylphosphino)ferrocene]-dichloropalladium(II),
V65 is 2,2′-azobis(2,4-dimethylvaleronitrile),
DIAD is diisopropyl azodicarboxylate,
SFC is super critical fluid chromatography,
AcOH is acetic acid,
Boc is tert-butyloxycarbonyl, DIPEA is diisopropylethylamine,
IBCF is isobutylchloroformate,
TFA is trifluoroacetic acid,
Brine is saturated aqueous sodium chloride solution,
Roshelles salt is potassium sodium tartrate,
LC-MS or LC/MS is liquid chromatography mass spectrometry,
LRMS is low resolution mass spectrometry,
HRMS is high resolution mass spectrometry,
ES+ is electron spray positive charge,
ES− is electron spray negative charge, and
RT is room temperature.

Compounds of the present invention can be prepared beginning with commercially available starting materials and utilizing general synthetic techniques and procedures known to those skilled in the art. Outlined below are reaction schemes suitable for preparing such compounds. Further exemplification is found in the specific examples listed in the later section.

The preparation of N-alkylamino acetic acid esters are described in Scheme 1, below. The substituted biarylphenol (ii) can be obtained through the coupling reaction of 4-hydroxyaryl boronic acid with substituted arylbromide (i) under the condition of palladium catalysis know as Suzuki coupling. Non-commercially available arylbromide (i) can be obtained by bromination reaction. Alternatively, biarylphenol (ii) can also be obtained through the coupling of substituted aryl boronic acid (iii) with 4-iodophenol under the same palladium catalyzed coupling conditions. The substituents of R1, R2 and R3 in compound (II) can be fluoro, chloro, methyl or methoxy group.

The biarylphenol (ii) can be alkylated with 3-cyanobenzylbromide under basic condition to provide biaryl ether (iv). Alternatively, the biaryl ether (iv) can also be obtained through the coupling of aryl iodide (v) with substituted aryl boronic acid (iii) under palladium catalyzed coupling conditions. Compound (v) can be prepared through the alkylation of 4-iodophenol with 3-cyanobenzylbromide under basic conditions. Reduction of the cyano group in biaryl ether (iv) can be achieved with lithium aluminum hydride to provide the corresponding primary amine (vi). Alkylation of biaryl ether derived amine (vi) with 2-bromoacetic acid esters under basic condition can provide the desired N-alkylated aminoacetic acid esters (vii). The R5 substituent in (vii) can be methyl, ethyl or tert-butyl groups.

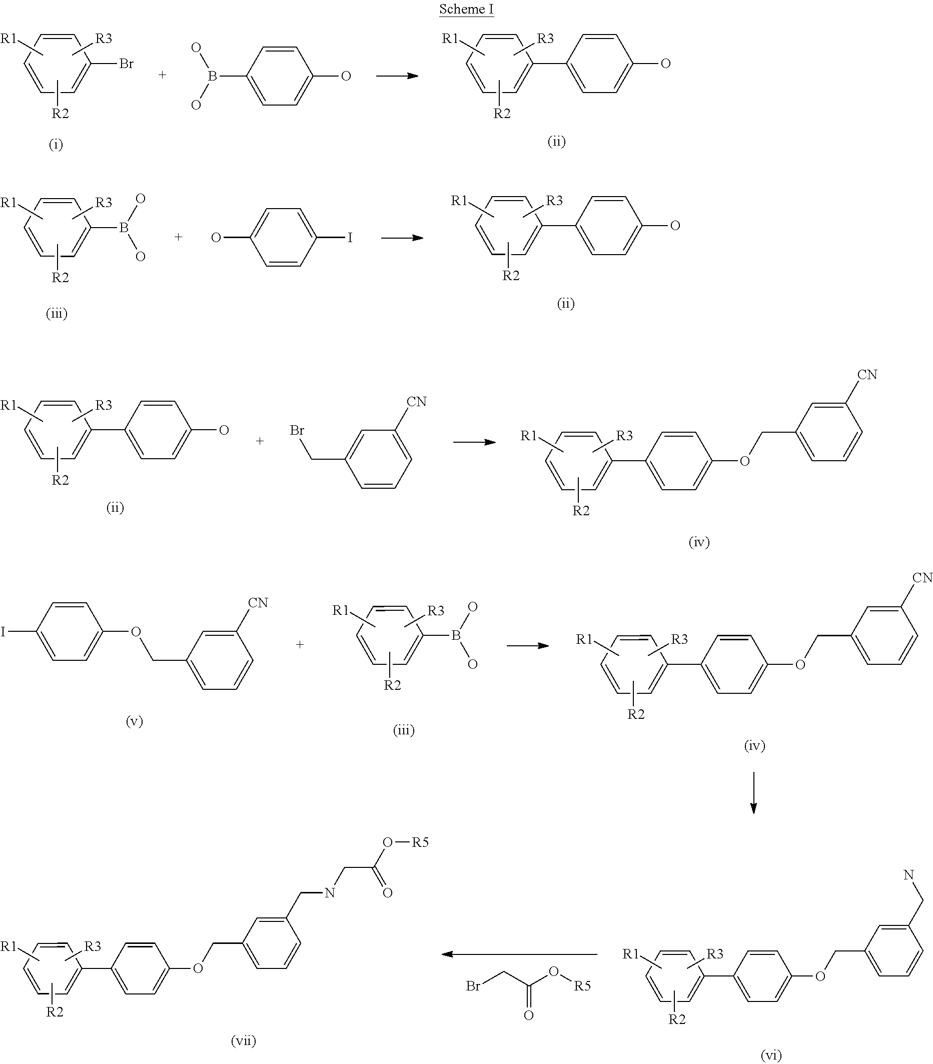

As described in Scheme 2, below, the biaryl ether derived amino acetic acid ester (vii) can be further functionalized. Reaction of compound (vii) with acyl chloride under basic condition can provide a corresponding amide. Saponification of the corresponding amide ester can give the desired GS activator compound (viii). Alternatively, GS activator (viii) can also be obtained by the coupling of compound (vii) with a carboxylic acid followed by saponification reaction. The coupling reaction can be accomplished with coupling reagents, such as BOP reagent, or other reagents used in peptide coupling reactions. The R6 in compound (viii) can be alkyl, substituted alkyl, cycloalkyl, heterocycloalkyl, aryl, and heteroaryl groups.

As described in Scheme 2, the reaction of compound (vii) with a chloroformate under basic conditions followed by saponification can give a corresponding carbamate (ix), where R7 in GS activator compound (ix) can be alkyl or substituted alkyl groups.

As described in Scheme 2, the reaction of compound (vii) with a sulfonyl chloride under basic conditions followed by saponification can give a corresponding sulfonamide (x), where R8 in GS activator compound (x) can be alkyl, substituted alkyl, cycloalkyl and aryl groups.

As described in Scheme 2, the reaction of compound (vii) with a dialkylaminocarbonyl chloride under basic conditions followed by saponification can give a corresponding urea (xi), where N9 and N10 in GS activator compound (xi) can be alkyl groups. N9 and N10 in compound (xi) can also form a ring or a heterocyclic ring to give a corresponding cyclic urea.

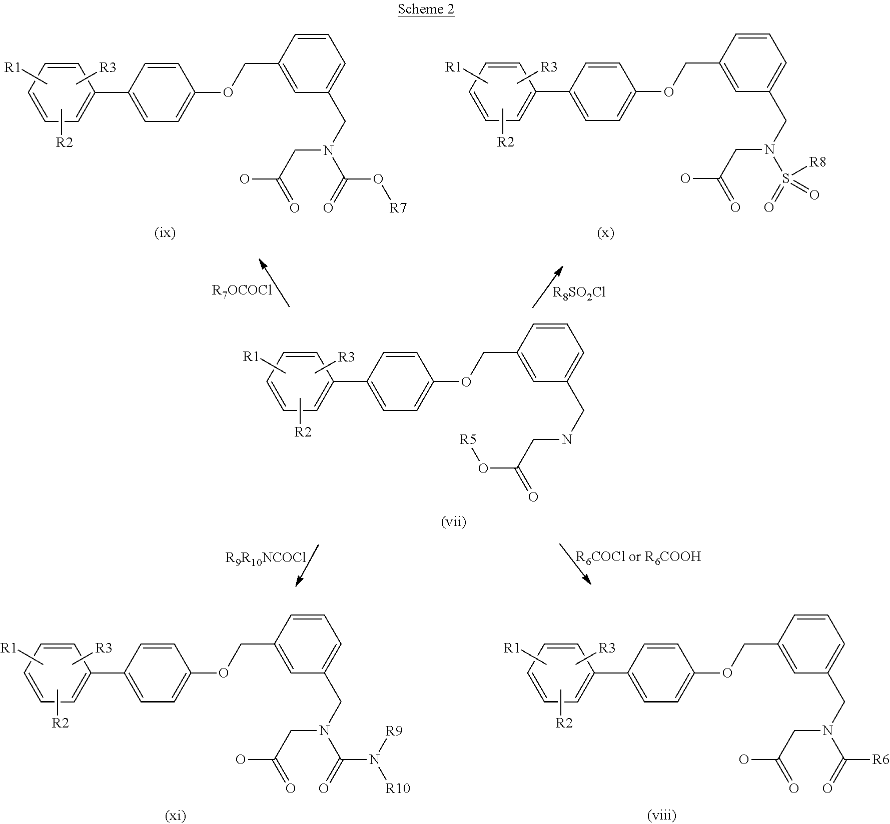

The preparation of biarylether derived benzaldehyde and benzyl bromide are described in Scheme 3, below. Biarylphenol (ii) can be alkylated with 3-bromomethylbenzaldehyde under basic conditions to provide a biarylether derived benzaldehyde (xii), where R1, R2 and R3 can be fluoro, chloro, methyl and methoxy groups. Reduction of the aldehyde (xii) can be achieved with lithium aluminum hydride or sodium borohydride to give the corresponding hydroxyl derivative (xiii). Conversion of hydroxyl group in (xiii) to a corresponding bromide (xiv) can be achieved with phosphorus tribromide.

The preparation of biaryl ether derived benzyl bromide (xix) with substituent R4, where R4 can be fluorine, is described in Scheme 3, below. Compound (xv) can be brominated with NBS under the catalysis of radical initiator, such as benzoylperoxide or V65, to provide the corresponding benzylbromide (xvi). Alkylation of compound (xvi) with substituted biaryl phenol (ii) under basic conditions can provide the corresponding biaryl ether (xvii). Reduction of the ester group in (xvii) can be achieved with lithium aluminum hydride to give a hydroxyl derivative (xviii). Conversion of the hydroxyl group in (xviii) to a corresponding bromide (xix) can be achieved with phosphorus tribromide.

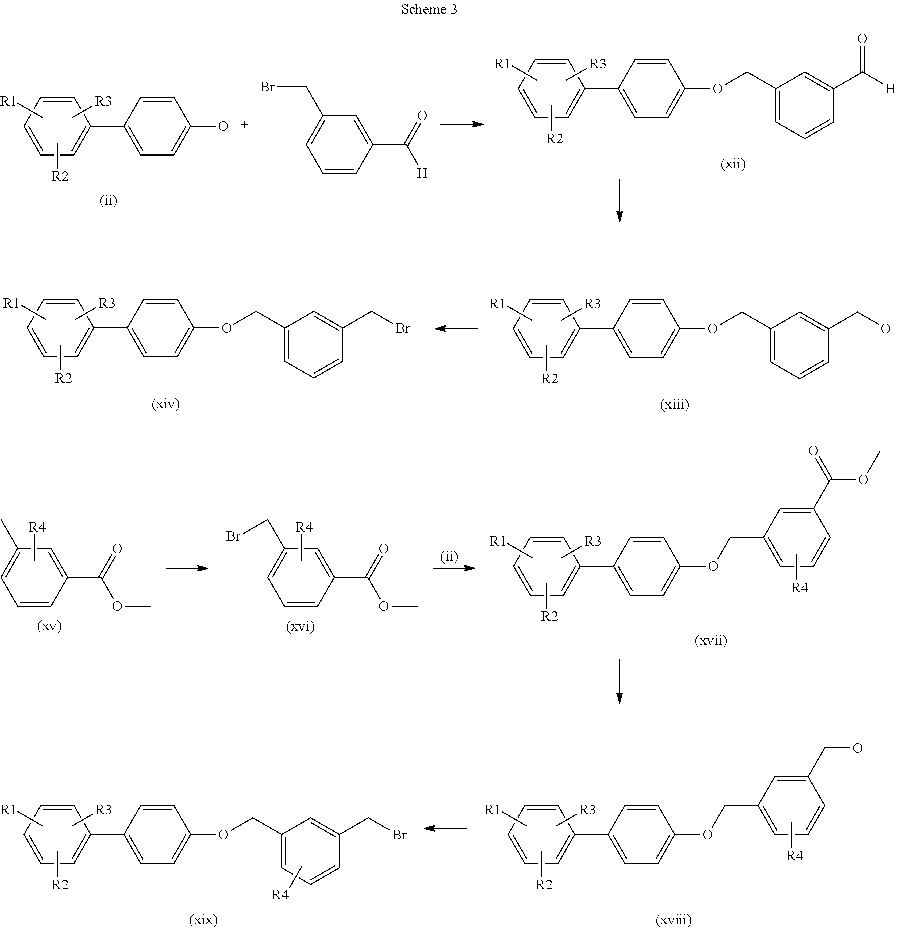

In order to prepare biaryl ether derived alkyl bromide containing thiazole and pyridine groups, reactions can be carried out according to Scheme 4, below. 2-Bromomethylthiazole-4-carboxylic acid ethyl ester (xx) can be obtained according to procedures described in WO2006/058648. Alkylation of compound (xx) with biaryl phenol (ii) under basic conditions can give thiazole derived biaryl ether (xxi). Reduction of ester group in (xxi) with reducing reagents such as lithium aluminum hydride can provide a hydroxyl derivative (xxii). Conversion of the hydroxyl group in (xxii) to a corresponding iodide (xxiii) can be achieved with iodine and triphenylphosphine under mild basic condition, where R1, R2 and R3 in compound (xxiii) can be fluoro, chloro, methyl or methoxy groups.

As described in Scheme 4, below, hydroxymethylpyridine derivative (xxiv) can be reacted with biaryl phenol (ii) in the presence of triphenylphosphine and DIAD to form the biaryl ether (xxv). Reduction of the ester group in (xxv) can be achieved with reducing reagents, such as lithium aluminum hydride, to provide the corresponding hydroxyl derivative (xxvi). Conversion of the hydroxyl derivative (xxvi) to the corresponding iodide (xxvii) can be accomplished with triphenylphosphine and iodine under mild basic conditions.

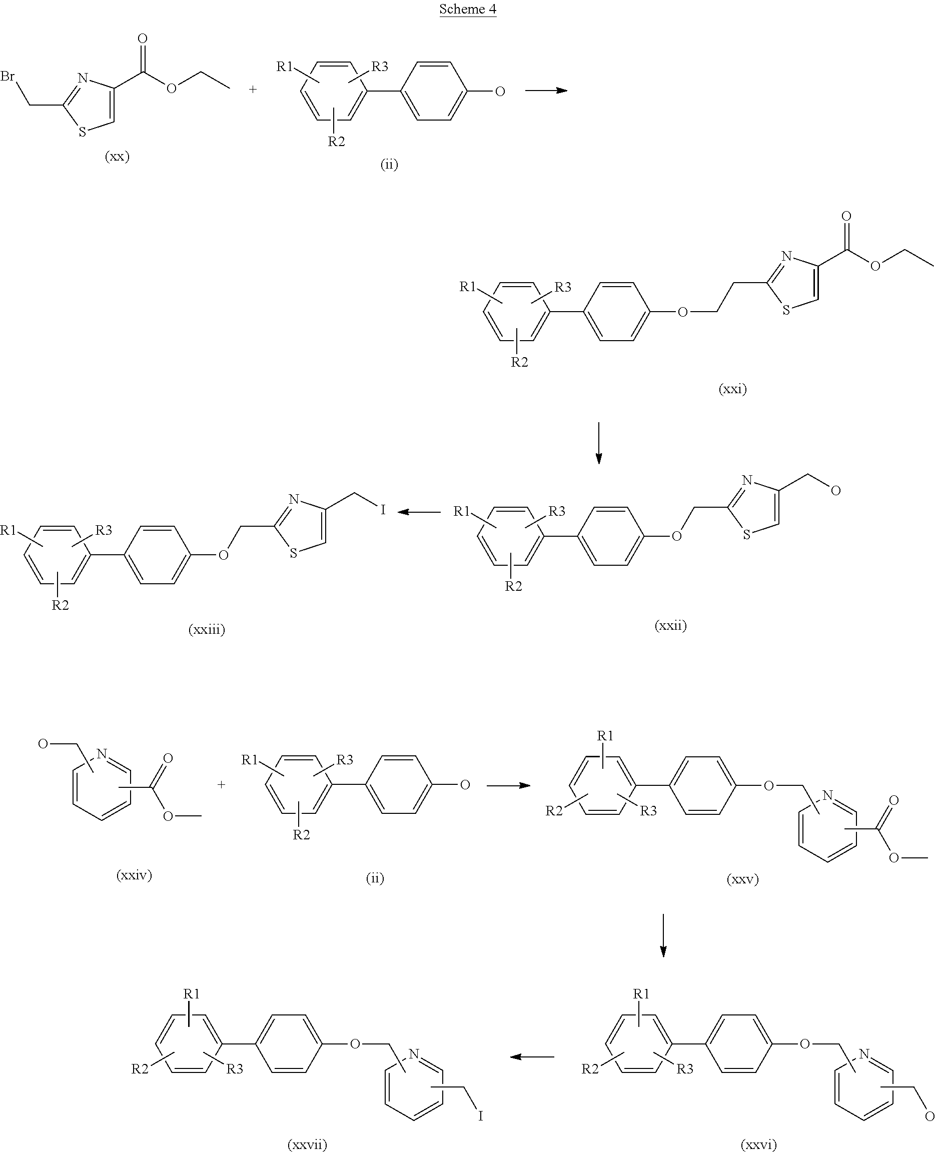

As described in Scheme 5, below, commercially available sultam (xxviii) can be N-alkylated with bromide derivative (xix) in the presence of base, such as potassium carbonate, to give the corresponding N-alkylated sultam (xxix). Saponification of sultam ester (xxix) in the presence of base, such as aqueous lithium hydroxide, can provide the desired GS activator (xxx), where R1, R2 and R3 can be fluoro, chloro, methyl and methoxy groups, R4 can be hydrogen or fluoro. The racemate (xxx) can be separated to (R) and (S) pure enantiomers on a chiral column using SFC. The chiral purity can be obtained by analyzing on a chiral column using SFC.

Alternatively, the pure enantiomer (xxxiii) can be obtained by using a chiral sultam (xxxi). Chiral sultam can be prepared according to Scheme 6. As described in Scheme 5, alkylation of (xix) with (S) or (R) sultam (xxxi) in the presence of base, such as potassium carbonate, can give a chirally pure (S) or (R) enantiomer (xxxii). Removal of the tert-butyl ester in (xxxii) in the presence of acid, such as trifluoroacetic acid, can provide the pure enantiomer (xxxiii), where R1, R2 and R3 can be fluoro, chloro, methyl and methoxy groups, R4 can be hydrogen or fluorine. Both (R) and (S) enantiomers of (xxxiii) can be prepared with the same method. The chiral purity can be obtained by analysis on a chiral column using SFC. The absolute stereochemistry from chiral SFC separation can be assigned by comparing chiral SFC analysis of GS activator (xxx) with GS activator (xxxiii).

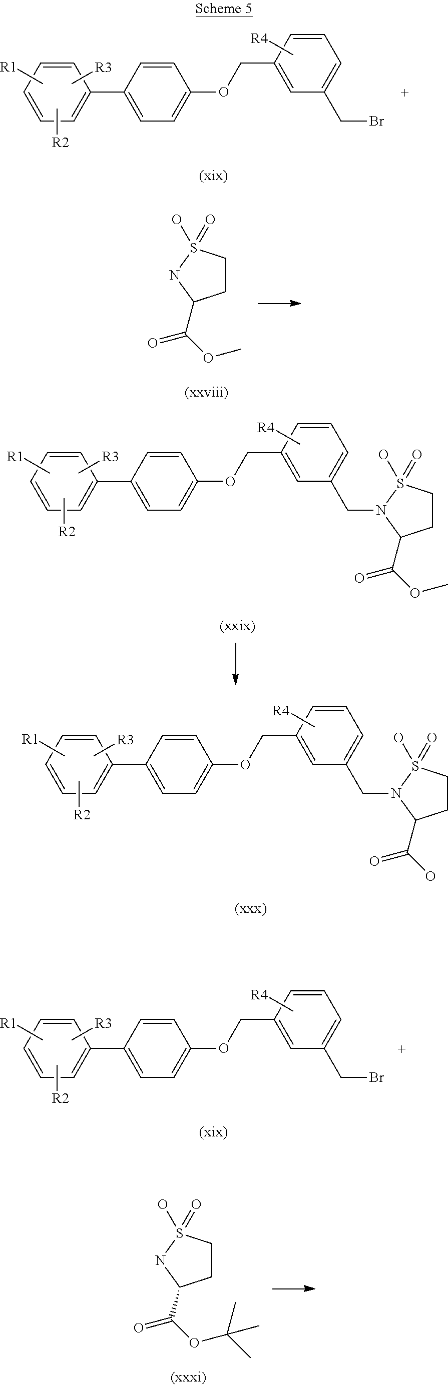

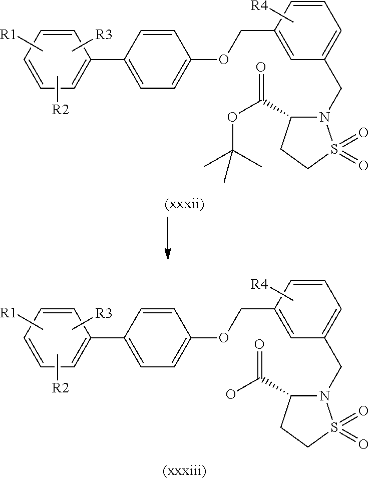

The preparation of both (R) and (S) sultam-3-tert-butyl ester can be carried out according to literature procedure (Journal of Medicinal Chemistry 2004, 47, 2981-2983; WO2002/028846).

Alternatively, the preparation of both (R) and (S) sultam-3-tert-butyl ester can also be achieved with an improved method as described in Scheme 6, below. The commercially available (D)-N-Boc-aspartic acid benzyl ester (xxxiv) can be esterified to form tert-butyl ester (xxxv) by reacting with tert-butanol in the presence of coupling reagent, such as DCC, as described in literature (Tetrahedron 2001, 57, 6557-6565). The benzyl group in (xxxv) can be removed under catalytic hydrogenation condition to give a corresponding acid (xxxvi). The carboxylic acid (xxxvi) can be first converted to a mixed anhydride in the presence of a chloroformate and then reduced to a corresponding alcohol (xxxvii) with reducing reagent, such as sodium borohydride, as described in literature (Australia Journal of Chemistry 1992, 45, 1225-1240). Formation of a thioester (xxxviii) can be achieved by reacting compound (xxxvii) with thioacetic acid in the presence of DIAD and triphenylphosphine as described in literature ((Journal of Medicinal Chemistry 2004, 47, 2981-2983; WO2002/028846). Oxidation of compound (xxxviii) with chlorine gas in aqueous acetic acid buffered with sodium acetate can give N-Boc-sultam-3-carboxylic acid tert-butyl ester (xxxix) with a very high yield. Selective removal of the N-Boc group in (xxxix) can be accomplished with trifluoroacetic acid to give a chiral sultam-3-tert-butyl ester (xxxi). Both (R) and (S) enantiomers can be prepared according to scheme 6.

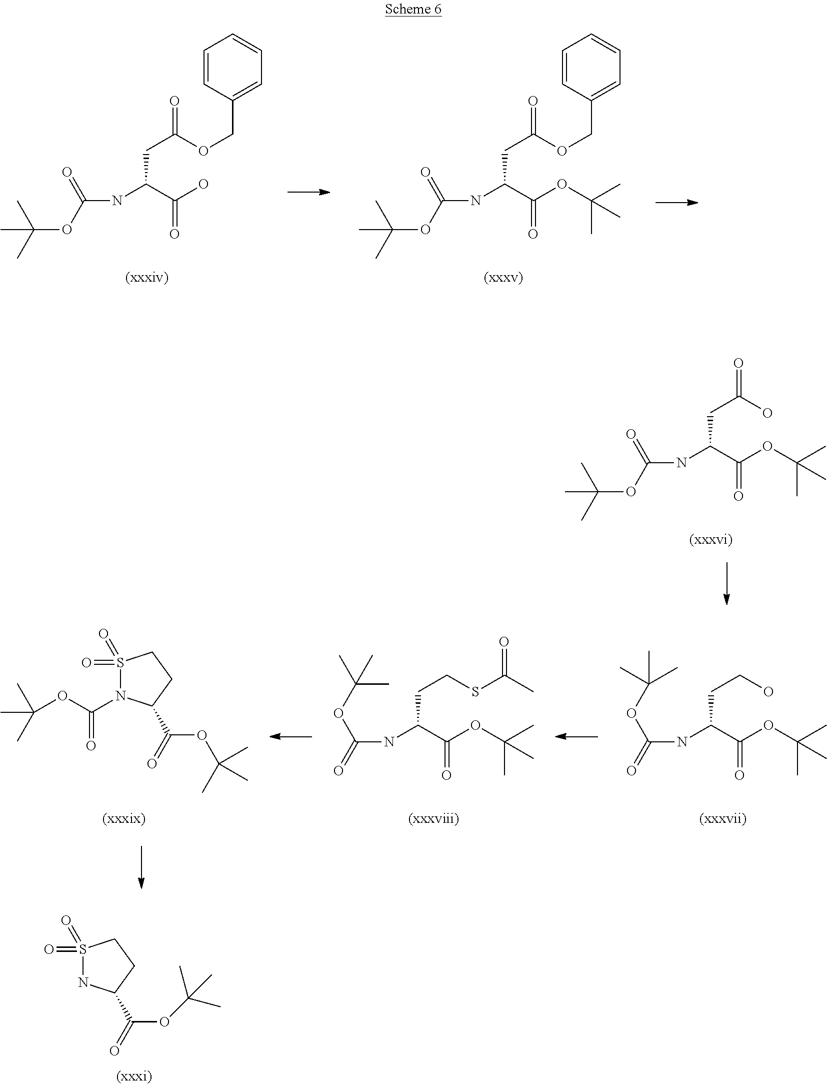

As described in scheme 7, below, the iodine derivative (xxiii) can be alkylated with sultam-3-carboxylic acid methyl ester (xxviii) in the presence of base, such as potassium carbonate, to give N-alkylated sultam derivative (xxxx). Saponification of (xxxx) can provide GS activator (xxxxi). Similarly, the iodine derivative (xxvii) in Scheme 7 can also react with sultam-3-carboxylic acid methyl ester (xxviii) in the presence of base, such as potassium carbonate to give pyridine derived N-alkylated sultam-3-carboxylic acid ester (xxxxii). Saponification of compound (xxxxii) can provide GS activator (xxxxiii). The racemate of (xxxxi) and (xxxxiii) can be separated on a chiral column using SFC to provide pure enantiomers.

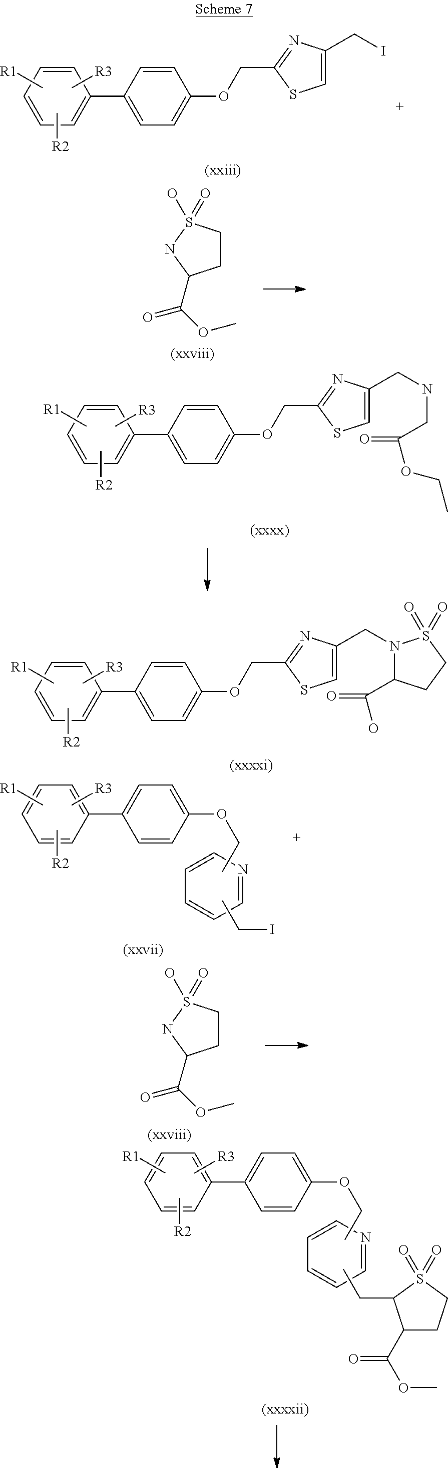

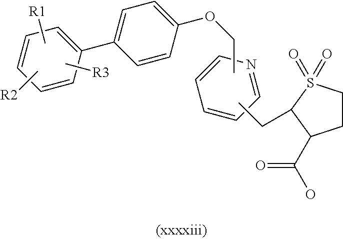

The intermediate 1,1-dioxo-[1,2]thiazinane-3-carboxylic acid tert-butyl ester (xxxxix) in Scheme 8, below, can be prepared according to literature procedure (Journal of Medicinal Chemistry 2004, 47, 2981-2983).

Alternatively, 1,1-dioxo-[1,2]thiazinane-3-carboxylic acid tert-butyl ester (xxxxix) can also be prepared with a modified method as described in Scheme 8. The commercially available pentanoic acid derivative (xxxxiv) can be converted to compound (xxxxv) first by ester formation with tert-butanol followed by catalytic hydrogenation to remove benzyl group. The tert-butyl ester formation can be achieved by reacting with tert-butanol in the presence of coupling reagent, such as DCC, as described in literature (Tetrahedron 2001, 57, 6557-6565). The carboxylic acid (xxxxv) can be first converted to a mixed anhydride in the presence of a chloroformate and then reduced to a corresponding alcohol (xxxxvi) with reducing reagent, such as sodium borohydride, as described in literature (Australia Journal of Chemistry 1992, 45, 1225-1240). Formation of a thioester (xxxxvii) can be achieved by reacting compound (xxxxvi) with thioacetic acid in the presence of DIAD and triphenylphosphine as described in literature (Journal of Medicinal Chemistry 2004, 47, 2981-2983; WO2002/028846). Oxidation of compound (xxxxvii) with chlorine gas in aqueous acetic acid buffered with sodium acetate can give compound (xxxxviii) with a very high yield. Selective removal of the N-Boc group in (xxxxviii) can be accomplished with trifluoroacetic acid to give a desired intermediate 1,1-dioxo-[1,2]thiazinane-3-carboxylic acid tert-butyl ester (xxxxix). Alkylation of intermediate (xxxxix) with bromide (xiv) in the presence of base, such as sodium carbonate, can provide N-alkylated compound (xxxxx). Cleavage of the tert-butyl ester in compound (xxxxx) under acidic conditions can give the desired GS activator (xxxxxi). The racemate (xxxxxi) can be separated on a chiral column with SFC to provide pure enantiomers.

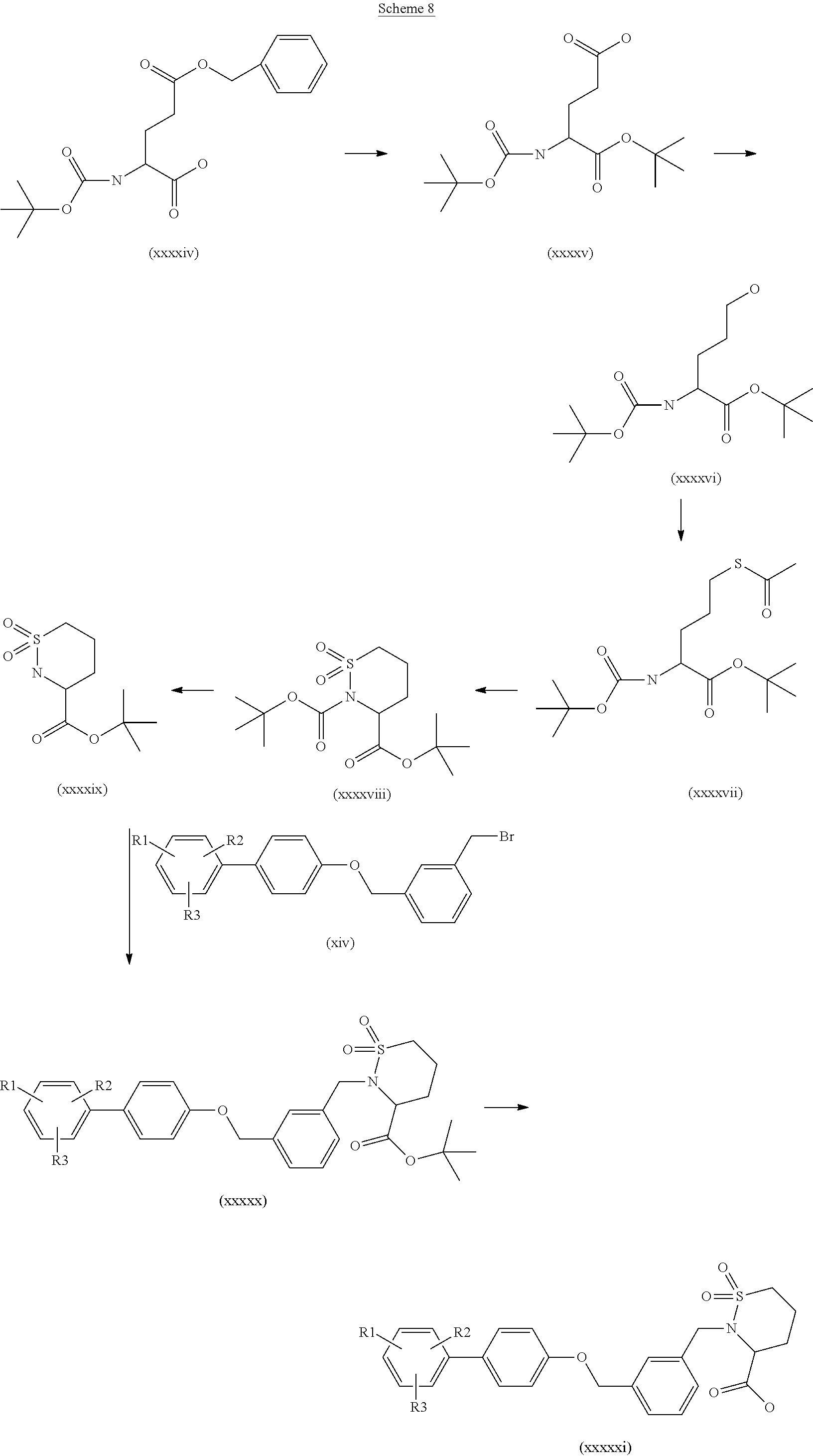

The preparation of biarylether derived pyrrolidinone carboxylic acid is described in Scheme 9, below. Commercially available (L)-glutamic acid di-ethyl ester (xxxxxii) can react with biaryl ether derived benzaldehyde (xii) under reductive amination conditions, such as sodium triacetoxyboronhydride, to form N-alkylated intermediate which can cyclize during the reaction to provide N-alkylated pyrrolidinone derivative (xxxxxiii). Saponification of (xxxxxiii) in the presence of base, such as lithium hydroxide, can give the desired GS activator (xxxxxiv).

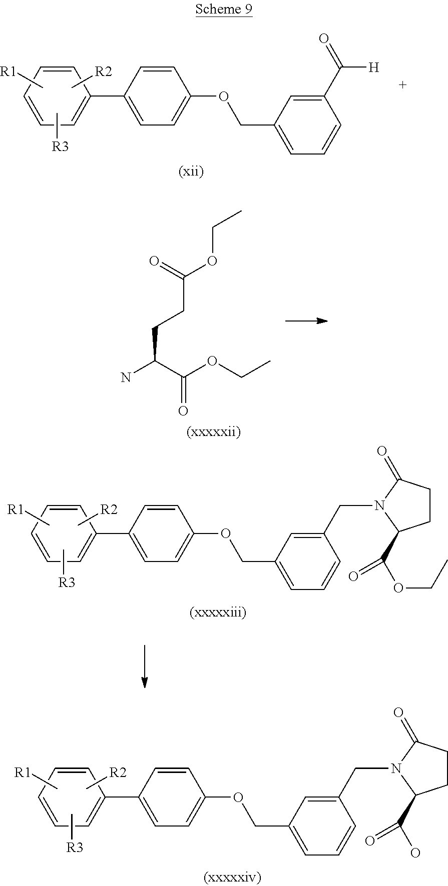

The invention will now be further described in the Examples below, which are intended as an illustration only and do not limit the scope of the invention.

### EXAMPLES

### Part I

### Preparation of Preferred Intermediates

### 2′,4′,5′-trifluoro-biphenyl-4-ol

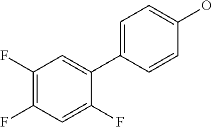

A mixture of 2,4,5-trifluorophenylboronic acid (1.00 g, 5.685 mmol), 4-iodophenol (1.4 g, 6.363 mmol), potassium carbonate (2.4 g, 17.36 mmol), tetrakis(triphenylphosphine) palladium (0) (165 mg, 0.143 mmol), DMF (15 mL) and water (4 mL) was heated at 70° C. for 2 h. The reaction mixture was cooled, diluted with water (50 mL) and extracted with ethyl acetate (3×50 mL). The organic layers were combined, washed with water (50 mL), brine (50 mL), dried (MgSO₄), filtered, concentrated, flash chromatographed (silica, 120 g, 15% ethyl acetate in hexanes) to give 2′,4′,5′-trifluoro-biphenyl-4-ol (1.04 g, 81.6%) as a gray solid. LC-MS (ES) calculated for C₁₂H₇F₃O, 224.18; found m/z 223 [M−H]⁻.

### 4′,5′-Difluoro-2′-methoxy-biphenyl-4-ol

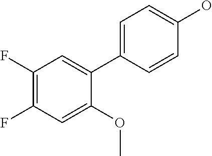

In a similar manner, from 4,5-difluoro-2-methoxyphenylboronic acid there was produced 4′,5′-difluoro-2′-methoxy-biphenyl-4-ol (98.7%) as a red oil. LC-MS (ES) calculated for C₁₃H₁₀F₂O₂, 236.22; found m/z 235 [M−H]⁻.

### 3-(4′,5′-Difluoro-2′-methoxy-biphenyl-4-yloxymethyl)-benzonitrile

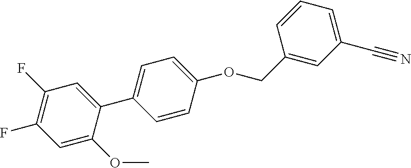

A mixture of 4-5-Difluoro-2-methoxyphenylboronic acid (5.0 g, 26.6 mmol), and 3-(4-iodo-phenoxymethyl)-benzonitrile (7.40 g, 22.2 mmol), potassium carbonate (9.20 g, 67.0 mmol), and [1,1′-bis(diphenylphosphino)ferrocene]-dichloropalladium(II) (550 mg, 0.7 mmol) in DMF (100 mL) and water (20 mL) was heated to 60° C. and stirred overnight. The reaction mixture was diluted with ethyl acetate and washed with water and brine. The solution was dried with anhydrous sodium sulfate and the solvent was removed. The residue was purified on a flash chromatography column with ethyl acetate in hexanes to afford 3-(4′,5′-difluoro-2′-methoxy-biphenyl-4-yloxymethyl)-benzonitrile (7.50 g, 96% yield) as a amorphous solid. LRMS calcd for C₂₁H₁₅F₂NO₂ (m/e) 351.11, obsd 350.2 (M−H, ES−).

### 3-(4′,5′-Difluoro-2′-methoxy-biphenyl-4-yloxymethyl)-benzylamine hydrochloride

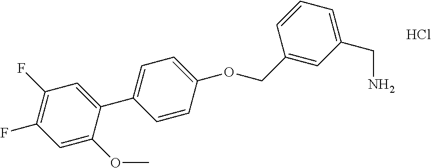

A solution of 3-(4′,5′-difluoro-2′-methoxy-biphenyl-4-yloxymethyl)-benzonitrile (7.4 g, 21.1 mmol) in THF (50 mL) was added slowly to a suspension of lithium aluminum hydride (2.0 g, 52.5 mmol) in ether (100 mL) at 0° C. The reaction mixture was allowed to reach ambient temperature and stirred for 2 h. The mixture was quenched with water (6 mL), 1N NaOH (2 mL) and stirred for 10 minutes. The mixture was filtered through a pad of celite and rinsed with THF. The filtrate was extracted with ethyl acetate and water. The organic layer was dried with sodium sulfate and solvents were evaporated. The residue was dissolved in THF (20 mL) and treated with HCl in dioxane (4M, 10 mL) and stirred for 10 minutes. The mixture was evaporated to dryness and the residue was triturated with ether. The precipitate was filtered and dried in a vacuum oven to afford 3-(4′,5′-difluoro-2′-methoxy-biphenyl-4-yloxymethyl)-benzylamine hydrochloride (7.00 g, 85% yield) as a light yellow solid. LRMS calcd for C₂₁H₁₉F₂NO₂ (m/e) 355.14, obsd 356.1 (M+H, ES+).

### [3-(4′,5′-Difluoro-2′-methoxy-biphenyl-4-yloxymethyl)benzylamino]-acetic acid ethyl ester hydrochloride

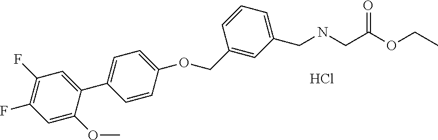

A mixture of 3-(4′,5′-difluoro-2′-methoxy-biphenyl-4-yloxymethyl)-benzylamine hydrochloride (3.97 g, 10.1 mmol), bromoacetic acid ethyl ester (1.80 g, 10.1 mmol) and triethylamine (3.04 g, 30.0 mmol) in acetonitrile (100 mL) was heated to 60° C. and stirred for 15 h. The reaction mixture was concentrated and diluted with ethyl acetate (200 mL), washed with saturated ammonium chloride solution (100 mL) twice. The organic layer was washed with water and brine, dried with anhydrous sodium sulfate and the solvent was removed. The residue was purified on a flash chromatography column with ethyl acetate in hexanes to produce clear oil. The oily product was converted to the hydrochloride salt with gaseous HCl in ether (3M, 4 mL) to afford [3-(4′,5′-difluoro-2′-methoxy-biphenyl-4-yloxymethyl)-benzylamino]-acetic acid ethyl ester hydrochloride (1.66 g, 42% yield) as a white solid. LRMS calcd for C₂₅H₂₅F₂NO₄ (m/e) 441.18, obsd 442.2 (M+H, ES+).

### [3-(4′,5′-Difluoro-2′-methoxy-biphenyl-4-yloxymethyl)-benzylamino]-acetic acid tert-butyl ester

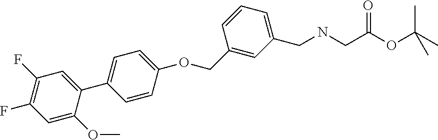

A mixture of 3-(4′,5′-difluoro-2′-methoxy-biphenyl-4-yloxymethyl)-benzylamine hydrochloride (2.09 g, 5.33 mmol), bromoacetic acid tert-butyl ester (1.05 g, 5.33 mmol) and triethylamine (1.90 g, 18.6 mmol) in acetonitrile (40 mL) was refluxed for 15 h. The reaction mixture was concentrated, diluted with ethyl acetate and washed with saturated ammonium chloride solution. The organic layer was washed with water and brine, dried with anhydrous sodium sulfate and the solvent was removed. The residue was purified on a flash chromatography column with ethyl acetate in hexanes to afford [3-(4′,5′-difluoro-2′-methoxy-biphenyl-4-yloxymethyl)-benzylamino]-acetic acid tert-butyl ester (1.10 g, 44% yield) as a clear oil. LRMS calcd for C₂₇H₂₉F₂NO₄ (m/e) 469.21, obsd 470.1 (M+H, ES+).

### [3-(2′,4′-Difluoro-biphenyl-4-yloxymethyl)-benzylamino]-acetic acid ethyl ester hydrochloride

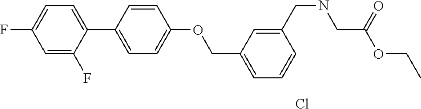

This compound was prepared using the same method as described for the preparation of [3-(4′,5′-difluoro-2′-methoxy-biphenyl-4-yloxymethyl)-benzylamino]-acetic acid ethyl ester hydrochloride. HRMS calcd for C₂₄H₂₃NO₃F₂ (m/e) 412.1719 (M+H), obsd 412.1717; ¹H-NMR (300 MHz, DMSO-d₆) δ ppm 1.22 (t, J=7.1 Hz, 3H), 3.94 (s, 2H), 4.19 (q, J=7.1 Hz, 2H), 4.19 (br s, 2H), 5.18 (s, 2H), 7.13 (d, J=8.8 Hz, 2H), 7.13-7.22 (m, 1H), 7.33 (ddd, J=11.2, 9.2, 2.6 Hz, 1H), 7.41-7.60 (m, 6H), 7.64 (s, 1H), 9.78 (br s, 2H).

### 3-(4′,5′-Difluoro-2′-methoxy-biphenyl-4-yloxymethyl)-benzaldehyde

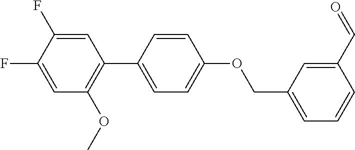

4′,5′-Difluoro-2′-methoxy-biphenyl-4-ol (6.05 g, 25.65 mmol) and 3-bromomethylbenzaldehyde (5.08 g, 25.65 mmol) was dissolved in acetone (100 mL) and potassium carbonate (5.3 g, 38.5 mmol) was added. The mixture was refluxed overnight and concentrated. The resulting mixture was treated with ethyl acetate (100 mL) and filtered. The filtrate was washed with water and brine, dried over sodium sulfate and solvents were evaporated. The resulting material was crystallized from ethyl acetate and hexanes to provide 3-(4′,5′-difluoro-2′-methoxy-biphenyl-4-yloxymethyl)-benzaldehyde as a light yellow solid (4.55 g, 50.1% yield). ¹H-NMR (300 MHz, DMSO-d₆) δ ppm 10.04 (s, 1H), 8.00 (s, 1H), 7.89 (d, J=7.5 Hz, 1H), 7.81 (d, J=7.8 Hz, 1H), 7.65 (dd, J=7.8, 7.5 Hz, 1H), 7.42 (d, J=8.8 Hz, 2H), 7.36 (dd, J=11.5, 9.4 Hz, 1H), 7.23 (dd, J=13.0, 7.2 Hz, 1H), 7.07 (d, J=8.8 Hz, 2H), 5.26 (s, 2H), 3.75 (s, 3H).

### 3-(2′,4′,5′-Trifluoro-biphenyl-4-yloxymethyl)-benzaldehyde

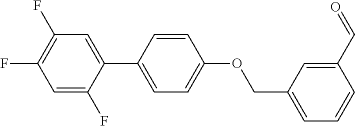

This compound was prepared with the same method as described for the preparation of 3-(4′,5′-difluoro-2′-methoxy-biphenyl-4-yloxymethyl)-benzaldehyde. ¹H-NMR (300 MHz, DMSO-d₆) δ ppm 10.04 (s, 1H), 8.01 (s, 1H), 7.90 (d, J=7.5 Hz, 1H), 7.79-7.84 (m, 1H), 7.58-7.73 (m, 3H), 7.51 (dd, J=8.5, 1.5 Hz, 2H), 7.15 (d, J=8.5 Hz, 2H), 5.29 (s, 2H).

### 4′-(3-Bromomethyl-benzyloxy)-4,5-difluoro-2-methoxy-biphenyl

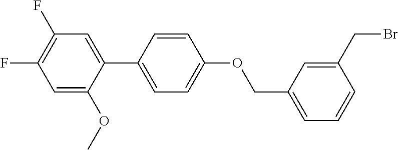

3-(4′,5′-difluoro-2′-methoxy-biphenyl-4-yloxymethyl)-benzaldehyde (1.06 g, 3.0 mmol) was dissolved in THF (30 mL). The solution was cooled in an ice bath and lithium aluminum hydride (120 mg, 3.2 mmol) was added. The mixture was stirred at room temperature for 1 hr and water (1 mL) was added. The mixture was stirred for 5 minutes and filtered through a pad of celite and washed with THF. The filtrate was concentrated and the residue was extracted with ethyl acetate and brine. The organic layer was dried and evaporated to give [3-(4′,5′-difluoro-2′-methoxy-biphenyl-4-yloxymethyl)-phenyl]-methanol (1.06 g, 100% yield). ¹H-NMR (300 MHz, CDCl₃) δ ppm 7.32-7.51 (m, 6H), 7.12 (t, J=10.0 Hz, 1H), 7.03 (d, J=8.5 Hz, 2H), 6.78 (dd, J=12.2, 6.8 Hz, 1H), 5.11 (s, 2H), 4.75 (s, 2H), 3.78 (s, 3H), 1.69 (br s, 1H).

[3-(4′,5′-difluoro-2′-methoxy-biphenyl-4-yloxymethyl)-phenyl]-methanol (1.05 g, 2.95 mmol) was dissolved in methylene chloride (15 mL) and phosphous tribromide (800 mg, 2.95 mmol) was added at 0° C. The solution was stirred at 0° C. for 1 hr and at room temperature for 5 hrs. The mixture was poured into ice water and extracted with methylene chloride. The organic layer was washed with water and concentrated. The residue was passed through a pad of silica gel and washed with ethyl acetate in hexanes (1:2 ratio). Solvents were evaporated to provide an oil as 4′-(3-bromomethyl-benzyloxy)-4,5-difluoro-2-methoxy-biphenyl (520 mg, 42% yield). ¹H-NMR (300 MHz, CDCl₃) δ ppm 7.35-7.55 (m, 6H), 7.12 (t, J=10.1 Hz, 1H), 7.02 (d, J=7.2 Hz, 2H), 6.79 (dd, J=11.8, 6.6 Hz, 1H), 5.10 (s, 2H), 4.53 (s, 2H), 3.78 (s, 3H).

### 4′-(3-Bromomethyl-benzyloxy)-2,4,5-trifluoro-biphenyl

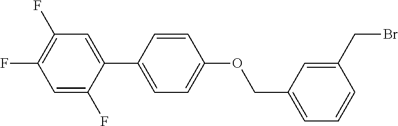

This compound was prepared with the same method as described for the preparation of 4′-(3-bromomethyl-benzyloxy)-4,5-difluoro-2-methoxy-biphenyl. ¹H-NMR (300 MHz, CDCl₃) δ ppm 7.50 (s, 1H), 7.37-7.48 (m, 5H), 7.16-7.26 (m, 1H), 7.06 (d, J=8.8 Hz, 2H), 6.93-7.04 (m, 1H), 5.11 (s, 2H), 4.53 (s, 2H).

### 4′-(3-Bromomethyl-benzyloxy)-4,5-difluoro-2-methyl-biphenyl

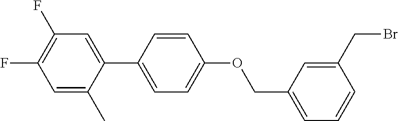

This compound was prepared with the same method as described for the preparation of 4′-(3-bromomethyl-benzyloxy)-4,5-difluoro-2-methoxy-biphenyl. ¹H NMR (300 MHz, DMSO-d₆) δ ppm 7.56 (s, 1H), 7.42 (s, 3H), 7.37 (dd, J=12.1, 8.8 Hz, 1H), 7.29 (d, J=8.5 Hz, 2H), 7.24 (dd, J=11.5, 8.5 Hz, 1H), 7.08 (d, J=8.5 Hz, 2H), 5.14 (s, 2H), 4.74 (s, 2H), 2.19 (s, 3H).

### 4′-(3-Bromomethyl-benzyloxy)-2-chloro-4,5-difluoro-biphenyl

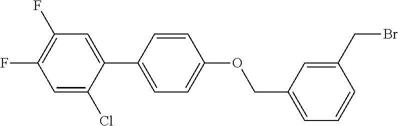

This compound was prepared with the same method as described for the preparation of 4′-(3-bromomethyl-benzyloxy)-4,5-difluoro-2-methoxy-biphenyl. ¹H NMR (300 MHz, DMSO-d₆) δ ppm 7.75-7.87 (m, 1H), 7.49-7.60 (m, 2H), 7.38 (d, J=8.5 Hz, 2H), 7.33-7.45 (m, 3H), 7.11 (d, J=8.8 Hz, 2H), 5.16 (s, 2H), 4.74 (s, 2H).

### 4′-(5-Bromomethyl-2-fluoro-benzyloxy)-2,4-difluoro-biphenyl

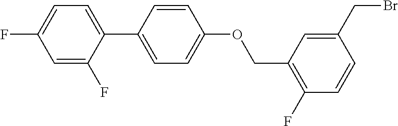

To a solution of 4-fluoro-3-methylbenzoic acid (5.0 g, 32.4 mmol) in methanol (150 mL) was added concentrated hydrochloric acid (2 mL). The mixture was refluxed for 4 hrs and then concentrated. The residue was treated with ether (200 mL) and washed with water, 10% sodium hydroxide solution, water and finally brine. The organic layer was dried over sodium sulfate and solvents were removed to give 4-fluoro-3-methylbenzoic acid methyl ester (4.56 g, 83.7%) as an off white solid.

4-Fluoro-3-methylbenzoic acid methyl ester (4.56 g, 2.71 mmol) was suspended in carbon tetrachloride (80 mL) and benzoyl peroxide (1.0 g, 75% by weight, 3.0 mmol) was added. The mixture was refluxed for 6 hrs and the solid was filtered. The filtrate was concentrated and purified through flash column chromatography (200 g silica gel, 0% to 15% ethyl acetate in hexanes over 50 minutes) to give 3-bromomethyl-4-fluorobenzoic acid methyl ester as a white solid (3.0 g, 45%).

3-Bromomethyl-4-fluorobenzoic acid methyl ester (3.0 g, 12.1 mmol) was mixed with 4-iodophenol (3.2 g, 14.6 mmol) in acetone (75 mL) containing dry potassium carbonate (2.5 g, 18 mmol). The mixture was refluxed for 6 hrs and then filtered. The filtrate was concentrated and extracted with ether and water. The organic layer was dried over sodium sulfate and solvents were evaporated. The residue was purified through flash column chromatography (120 g silica gel, 0% to 20% ethyl acetate in hexanes over 40 minutes) to give clear oil as 4-fluoro-3-(4-iodophenoxy)methylbenzoic acid methyl ester (3.48 g, 74.4%).

To a mixture of 4-fluoro-3-(4-iodophenoxy)methylbenzoic acid methyl ester (1.04 g, 2.7 mmol) and 2,4-difluorophenylboronic acid (0.553 g, 3.5 mmol) in DMF (30 mL) and water (3 mL) was added Pd(dppf)Cl₂ (220 mg, 0.27 mmol) and potassium carbonate (0.83 g, 6.0 mmol). The mixture was stirred at 45° C. overnight and solvents were evaporated. The residue was extracted with ethyl acetate and water. The organic layer was dried and concentrated. The residue was purified through flash column chromatography (80 g silica gel, 0% to 20% ethyl acetate in hexanes over 20 minutes) to give 3-(2′,4′-difluorobiphenyl-4-yloxymethyl)-4-fluorobenzoic acid methyl ester (0.8 g, 80%).

3-(2′,4′-Difluorobiphenyl-4-yloxymethyl)-4-fluorobenzoic acid methyl ester (0.8 g, 2.1 mmol) was dissolved in THF (35 mL) and lithium hydroxide solution (0.5N, 8 mL) was added. The mixture was refluxed for 6 hrs and concentrated. The residue was treated with hydrochloric acid (1N, 10 mL) and extracted with ethyl acetate. The organic layer was washed with water and dried over sodium sulfate. Solvent was evaporated to give 3-(2′,4′-difluorobiphenyl-4-yloxymethyl)-4-fluorobenzoic acid (0.75 g, 94%).

3-(2′,4′-Difluorobiphenyl-4-yloxymethyl)-4-fluorobenzoic acid (537 mg, 1.5 mmol) was dissolved in THF (50 mL) and borane in THF (1M, 3.0 mL) was added at 0° C. The mixture was stirred at 0° C. for 20 minutes and at room temperature for 4 hrs until all carboxylic acid was consumed according LC/MS analysis. The mixture was treated with methanol (10 mL) and solvents were evaporated. The residue was extracted with ethyl acetate and 1N hydrochloric acid. The organic layer was washed with brine and concentrated. The residue was crystallized from ethyl acetate and hexanes to give [3-(2′,4′-difluorobiphenyl-4-yloxymethyl)-4-fluoro-phenyl]-methanol (370 mg, 72%).

[3-(2′,4′-difluorobiphenyl-4-yloxymethyl)-4-fluoro-phenyl]-methanol (370 mg, 1.07 mmol) was dissolved in methylene chloride (15 mL) and phosphous tribromide in DCM (1.3 mL, 1.2 eq) was added at 0° C. The solution was stirred at 0° C. for 1 hr and at room temperature for 5 hrs. The mixture was poured into ice water and extracted with methylene chloride. The organic layer was washed with water and concentrated. The residue was purified through flash column chromatography (80 g silica gel, 0% to 40% ethyl acetate in hexanes) to provide 4′-(5-bromomethyl-2-fluoro-benzyloxy)-2,4-difluoro-biphenyl (300 mg, 68.6% yield). ¹H-NMR (300 MHz, CDCl₃) δ ppm 7.58 (d, J=6.3 Hz, 1H), 7.31-7.51 (m, 4H), 7.07 (d, J=8.5 Hz, 3H), 6.84-7.00 (m, 2H), 5.16 (s, 2H), 4.50 (s, 2H) 4′-(3-Bromomethyl-4-fluoro-benzyloxy)-2,4-difluoro-biphenyl

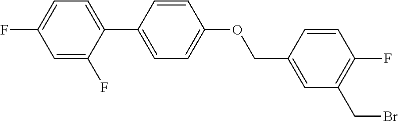

This compound was prepared with the same method as described for the preparation of 4′-(5-bromomethyl-2-fluoro-benzyloxy)-2,4-difluoro-biphenyl. ¹H-NMR (300 MHz, CDCl₃) δ ppm 7.31-7.55 (m, 5H), 7.00-7.19 (m, 3H), 6.84-6.99 (m, 2H), 5.06 (br. s., 2H), 4.54 (br. s., 2H)

### 2-(2′,4′,5′-Trifluoro-biphenyl-4-yloxymethyl)-thiazole-4-carboxylic acid ethyl ester

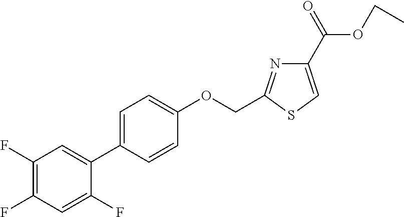

A mixture of 2′,4′,5′-trifluoro-biphenyl-4-ol (1.04 g, 4.639 mmol), 2-bromomethyl-thiazole-4-carboxylic acid ethyl ester (prepared according to patent US 2004/0266856 A1) (1.2 g, 4.798 mmol), potassium carbonate (2.6 g, 18.81 mmol) and potassium iodide (438 mg, 2.638 mmol) in DMF (12 mL) was heated by microwave at 130° C. for 15 min. The reaction mixture was diluted with water (100 mL) and extracted with ethyl acetate (3×75 mL). The organic layers were combined, washed with brine (50 mL), dried (MgSO₄), filtered and concentrated to give crude 2-(2′,4′,5′-trifluoro-biphenyl-4-yloxymethyl)-thiazole-4-carboxylic acid ethyl ester (1.74 g) as a tan solid which was used as is in the next step. LC-MS (ES) calculated for C₁₉H₁₄F₃NO₃S, 393.39; found m/z 394 [M+H]⁺.

### 2-(4′,5′-Difluoro-2′-methoxy-biphenyl-4-yloxymethyl)-thiazole-4-carboxylic acid ethyl ester

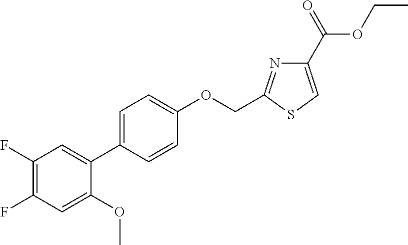

In a similar manner, from 4′,5′-difluoro-2′-methoxy-biphenyl-4-ol there was produced 2-(4′,5′-difluoro-2′-methoxy-biphenyl-4-yloxymethyl)-thiazole-4-carboxylic acid ethyl ester (89.5%) as an orange oil. LC-MS (ES) calculated for C₂₀H₁₇F₂NO₄S, 405.42; found m/z 406 [M+H]⁺.

### 5-(4′,5′-Difluoro-2′-methoxy-biphenyl-4-yloxymethyl)-nicotinic acid methyl ester

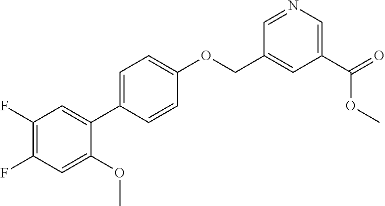

To a mixture of 4′,5′-difluoro-2′-methoxy-biphenyl-4-ol (683 mg, 2.89 mmol), methyl 5-(hydroxymethyl)nicotinate (484 mg, 2.9 mmol) and triphenylphosphine (1.14 g, 4.34 mmol) in THF (50 mL) was added dropwise diisopropyl azodicarboxylate (0.84 mL, 4.34 mmol) and stirred at room temperature for 4 h. The reaction mixture was diluted with ethyl acetate (100 mL), washed with 1/1 water/brine (50 mL), brine (50 mL), dried (MgSO₄), filtered, concentrated, flash chromatographed (silica, 120 g, 40% to 50% ethyl acetate in hexanes) to give 5-(4′,5′-difluoro-2′-methoxy-biphenyl-4-yloxymethyl)-nicotinic acid methyl ester (563.9 mg, 50.6%) as a white solid. LC-MS (ES) calculated for C₂₁H₁₇F₂NO₄, 385.37; found m/z 386 [M+H]⁺.

### 4-(4′,5′-Difluoro-2′-methoxy-biphenyl-4-yloxymethyl)-pyridine-2-carboxylic acid ethyl ester

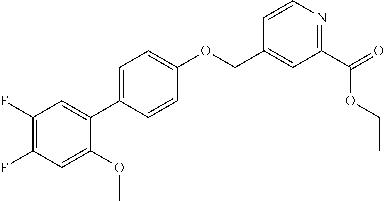

In a similar manner, from 4′,5′-difluoro-2′-methoxy-biphenyl-4-ol there was produced 4-(4′,5′-difluoro-2′-methoxy-biphenyl-4-yloxymethyl)-pyridine-2-carboxylic acid ethyl ester (26.8%) as a white solid. LC-MS (ES) calculated for C₂₂H₁₉F₂NO₄, 399.40; found m/z 400 [M+H]⁺.

### [2-(4′,5′-Difluoro-2′-methoxy-biphenyl-4-yloxymethyl)-thiazol-4-yl]-methanol

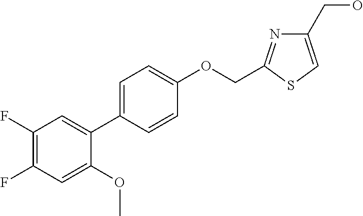

A solution of 2-(4′,5′-difluoro-2′-methoxy-biphenyl-4-yloxymethyl)-thiazole-4-carboxylic acid ethyl ester (0.63 g, 1.554 mmol) in tetrahydrofuran (14 mL) was treated with lithium borohydride (0.14 g, 6.428 mmol), stirred under nitrogen for 2 h, added additional lithium borohydride (0.035 g, 1.607 mmol) and heated at 60° C. for 1 h. The reaction mixture was cooled to room temperature, cautiously treated with water (3 mL) with vigorous gas evolution, added additional water (25 ml) and heated at 60° C. for 20 min. The reaction mixture was cooled and extracted with ethyl acetate (3×25 mL). The organic layers were combined, washed with 1/1 water/brine (30 mL), brine (30 mL), dried (MgSO₄), filtered and concentrated to give [2-(4′,5′-difluoro-2′-methoxy-biphenyl-4-yloxymethyl)-thiazol-4-yl]-methanol (494 mg, 87.5%) as an off white solid. LC-MS (ES) calculated for C₁₈H₁₅F₂NO₃S, 363.39; found m/z 364 [M+H]⁺.

### [2-(2′,4′,5′-Trifluoro-biphenyl-4-yloxymethyl)-thiazol-4-yl]methanol

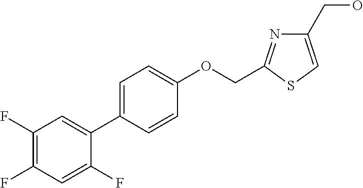

In a similar manner, from 2-(2′,4′,5′-trifluoro-biphenyl-4-yloxymethyl)-thiazole-4-carboxylic acid ethyl ester there was produced [2-(2′,4′,5′-trifluoro-biphenyl-4-yloxymethyl)-thiazol-4-yl]-methanol (93%) as a pale yellow solid. LC-MS (ES) calculated for C₁₇H₁₂F₃NO₂S, 351.35; found m/z 352 [M+H]⁺.

### [4-(4′,5′-Difluoro-2′-methoxy-biphenyl-4-yloxymethyl)-pyridin-2-yl]-methanol

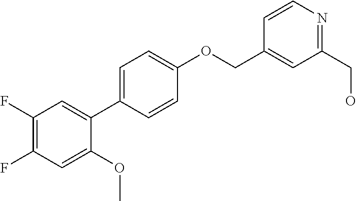

From 4-(4′,5′-difluoro-2′-methoxy-biphenyl-4-yloxymethyl)-pyridine-2-carboxylic acid ethyl ester (0.22 g) there was produced [4-(4′,5′-difluoro-2′-methoxy-biphenyl-4-yloxymethyl)-pyridin-2-yl]-methanol (0.110 g, 55.9%) as a white solid. LC-MS (ES) calculated for C₂₀H₁₇F₂NO₃, 357.36; found m/z 358 [M+H]⁺.

### [5-(4′,5′-Difluoro-2′-methoxy-biphenyl-4-yloxymethyl)-pyridin-3-yl]-methanol

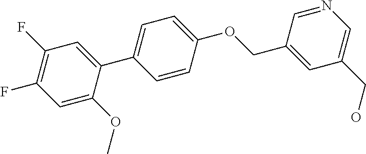

To a solution of 5-(4′,5′-difluoro-2′-methoxy-biphenyl-4-yloxymethyl)-nicotinic acid methyl ester (445 mg, 1.15 mmol) in THF (25 mL) stirred at 0° C. was added a 2.0M lithium aluminum hydride in THF solution (0.64 mL, 1.28 mmol) dropwise and the mixture was stirred at 0° C. for 20 min. The reaction mixture was cautiously diluted with Roshelles salt (15 mL) and extracted with ethyl acetate (75 mL). The organic layer was washed with water (25 mL), brine (25 mL), dried (MgSO₄), filtered and concentrated to give [5-(4′,5′-difluoro-2′-methoxy-biphenyl-4-yloxymethyl)-pyridin-3-yl]-methanol (0.37 g, 89.7%) as a colorless oil. LC-MS (ES) calculated for C₂₀H₁₇F₂NO₃, 357.36; found m/z 358 [M+H]⁺.

### 4-Iodomethyl-2-(2′,4′,5′-trifluoro-biphenyl-4-yloxymethyl)-thiazole

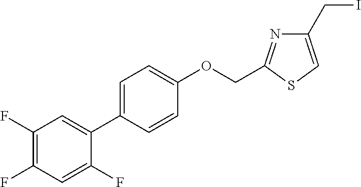

A mixture of [2-(2′,4′,5′-trifluoro-biphenyl-4-yloxymethyl)-thiazol-4-yl]-methanol (383 mg, 1.09 mmol,), triphenylphosphine (315 mg, 1.2 mmol) and imidazole (111 mg, 1.64 mmol) in tetrahydrofuran (3 ml) was treated with iodine (304 mg, 1.2 mmol) and the reaction mixture was stirred at room temperature for 45 min. The reaction mixture was diluted with water and ethyl acetate and extracted with ethyl acetate (3×). The organic layers were combined, washed with brine, dried over MgSO₄, filtered, concentrated, flash chromatographed (silica, 40 g, 15% to 25% ethyl acetate in hexanes) to give 4-iodomethyl-2-(2′,4′,5′-trifluoro-biphenyl-4-yloxymethyl)-thiazole (377.3 mg, 75%) as a white solid. LC-MS (ES) calculated for C₁₇H₁₁F₃INOS, 461.25; found m/z 462 [M+H]⁺.

### 2-(4′,5′-Difluoro-2′-methoxy-biphenyl-4-yloxymethyl)-4-iodomethyl-thiazole

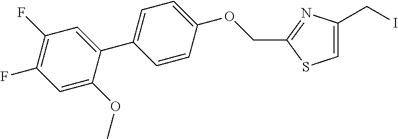

In a similar manner, from [2-(4′,5′-difluoro-2′-methoxy-biphenyl-4-yloxymethyl)-thiazol-4-yl]methanol (0.25 g) there was produced 2-(4′,5′-difluoro-2′-methoxy-biphenyl-4-yloxymethyl)-4-iodomethyl-thiazole (0.49 g, 77.7%) as a yellow oil. LC-MS (ES) calculated for C₁₈H₁₄F₂INO₂S, 473.28; found m/z 474 [M+H]⁺.

### 3-(4′,5′-Difluoro-2′-methoxy-biphenyl-4-yloxymethyl)-5-iodomethyl-pyridine

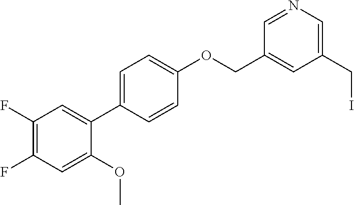

In a similar manner, from [5-(4′,5′-difluoro-2′-methoxy-biphenyl-4-yloxymethyl)-pyridin-3-yl]-methanol (0.24 g) there was produced 3-(4′,5′-difluoro-2′-methoxy-biphenyl-4-yloxymethyl)-5-iodomethyl-pyridine (0.31 g, 99%).

### 4-(4′,5′-Difluoro-2′-methoxy-biphenyl-4-yloxymethyl)-2-iodomethyl pyridine

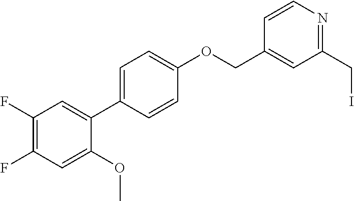

From [4-(4′,5′-difluoro-2′-methoxy-biphenyl-4-yloxymethyl)-pyridin-2-yl]-methanol (0.11 g) there was produced 4-(4′,5′-difluoro-2′-methoxy-biphenyl-4-yloxymethyl)-2-iodomethyl pyridine (0.07 g. 48.4%) as a red oil. LC-MS (ES) calculated for C₂₀H₁₆F₂₁NO₂, 467.26; found m/z 468 [M+H]⁺.

### Ethanesulfonylaminoacetic acid ethyl ester

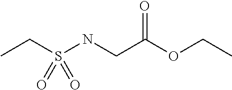

This compound was prepared from ethanesulfonyl chloride and aminoacetic acid ethyl ester hydrochloride according the procedure described in Journal of Combinatorial Chemistry 7 (3), 360-363, 2005. ¹H NMR (300 MHz, DMSO-d₆) δ ppm 7.58 (t, J=6.0 Hz, 1H), 4.10 (q, J=7.2 Hz, 2H), 3.77 (d, J=6.0 Hz, 2H), 3.01 (q, J=7.2 Hz, 2H), 1.19 (t, J=7.2 Hz, 3H), 1.18 (t, J=7.2 Hz, 3H).

### (R)-1,1-Dioxo-isothiazolidine-3-carboxylic acid tert-butyl ester

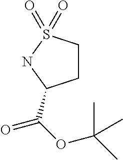

(R)-2-tert-butoxycarbonylamino-4-acetylsulfanyl-butyric acid tert-butyl ester (15.17 g, prepared according to procedure described in Journal of Medicinal Chemistry 2004, 47, 2981-2983, and WO2002/028846) was suspended in acetic acid (253 mL) and water (51 mL). At 5° C., sodium acetate (36.7 g, 447.5 mmol) was added to the stirred suspension. Chlorine gas was slowly bubbled to the stirred mixture (20.1 g of chlorine was absorbed). The mixture was bubbled with argon to remove excess chlorine and then evaporated under reduced pressure. The residue was extracted with ethyl acetate and water. The organic layer was washed with water and brine, dried over sodium sulfate. Solvents were evaporated and the residue was diluted with toluene (30 mL) and evaporated. The residue was dried in vacuum to give a white solid (14.62 g, 100% yield) as (R)-1,1-dioxo-2-N-tert-butoxycarbonylamino-isothiazolidine-3-carboxylic acid tert-butyl ester. ¹H NMR (300 MHz, chloroform-d) δ ppm 4.94 (dd, J=10.4, 5.0 Hz, 1H), 3.77 (t, J=7.5 Hz, 2H), 2.64-2.81 (m, 1H), 2.46-2.61 (m, 1H), 1.52 (s, 9H), 1.49 (s, 9H).

(R)-1,1-Dioxo-2-N-tert-butoxycarbonylamino-isothiazolidine-3-carboxylic acid tert-butyl ester (14.62 g) was dissolved in dichloromethane (400 mL) and trifluoroacetic acid (40 mL) was added at 0° C. The mixture was stirred at room temperature for 3.5 hr and then diluted with ethyl acetate (200 mL) and toluene (200 mL). Solvents were evaporated and the residue was extracted with ethyl acetate and dilute sodium bicarbonate solution. The organic layer was washed with brine and dried over sodium sulfate. Solvents were removed and the residue was purified through flash column chromatography (silica gel 200 g, 5% to 50% ethyl acetate in hexanes) to give a white solid as (R)-1,1-dioxo-isothiazolidine-3-carboxylic acid tert-butyl ester (5.0 g, 50% yield). ¹H NMR (300 MHz, chloroform-d) δ ppm 4.88 (br. s., 1H), 4.05 (dt, J=8.3, 4.3 Hz, 1H), 3.10-3.23 (m, 1H), 2.70-2.99 (m, 2H), 2.46-2.58 (m, 1H), 1.50 (s, 9H).

### (S)-1,1-Dioxo-isothiazolidine-3-carboxylic acid tert-butyl ester

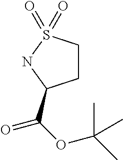

This compound was prepared with a similar method described in the literature (Journal of Medicinal Chemistry 2004, 47, 2981-2983). ¹H NMR (300 MHz, chloroform-d) δ ppm 4.88 (br. s., 1H), 3.98-4.13 (m, 1H), 3.11-3.24 (m, 1H), 2.69-2.97 (m, 2H), 2.52 (m, 1H), 1.50 (s, 9 H).

### 1,1-Dioxo-[1,2]thiazinane-3-carboxylic acid tert-butyl ester

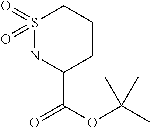

This compound was prepared with a modified method described in the Journal of Medicinal Chemistry 2004, 47, 2981-2983. 2-tert-Butoxycarbonylamino-pentanedioic acid 5-benzyl ester (10 g, 29.6 mmol) was suspended in 80 ml of methylene chloride, 2-methyl-propan-2-ol (6.6 g, 88.9 mmol) was added and the reaction was cooled on ice-water bath. Dicyclohexylcarbodiimide (7.2 g, 34.4 mmol) was added slowly and followed by 4-dimethylaminopyridine (362 mg, 3.0 mmol). The reaction was stirred at room temperature for 1 hour. White solid was filtered and the filter cake was washed with methylene chloride. The filtrate was concentrated and the residue was treated with ether. After the solid was filtered off, the ether solution was washed with 0.5N HCl solution, brine and concentrated. The crude product was purified by using an ISCO (200 g) column chromatography, eluting with 5-20% ethyl acetate in hexanes to obtain 2-tert-butoxycarbonylamino-pentanedioic acid 5-benzyl ester 1-tert-butyl ester as a white solid. (8.1 g, 69.5%). LC-MS (ES) calculated for C₂₁H₃₁NO₆, 393.5; found m/z 394.2 [M+H]⁺.

2-tert-Butoxycarbonylamino-pentanedioic acid 5-benzyl ester 1-tert-butyl ester (8.6 g, 21.9 mmol) was suspended in 100 mL of ethanol. Pd/C (2.3 g, 2.2 mmol) catalyst was added and the reaction was hydrogenated at 45 psi for 2 hours. The catalyst was filtered off and the filtrate was concentrated and the crude product was purified by using an ISCO (200 g) column chromatography, eluting with 5-20% ethyl acetate in hexanes to obtain 2-tert-butoxycarbonylamino-pentanedioic acid 1-tert-butyl ester as a white solid (4.2 g, 63.3%). LC-MS (ES) calculated for C₁₄H₂₅NO₆, 303.4; found m/z 304.3 [M+H]⁺.

2-tert-Butoxycarbonylamino-pentanedioic acid 1-tert-butyl ester (3.88 g, 12.8 mmol) was dissolved in 17 mL of THF. After cooled to −20° C., N-methylmorpholine (1.3 g, 12.8 mmol) in 12 mL THF solution was added followed by slowly addition of IBCF (12.8 mmol). The resulted suspension was stirred for 10 min and solid was filtered quickly. The filter cake was rinsed with cold THF (60 mL) and all filtrate was transferred to 250 mL round bottom flask which was cooled on ice-water bath. After the addition of sodium borohydride (968 mg, 25.6 mmol), methanol (13.4 ml) was added dropwise over 1 hour time period and the mixture was stirred for 1 hour on ice-water bath. While it is cold, ether (30 mL) and 30 mL of HCl (1.0N) solution was added, the organic layer was washed with saturated sodium bicarbonate, brine, dried and concentrated. The oil residue was purified by using an ISCO (120 g) column chromatography, eluting with 5-35% ethyl acetate in hexanes to obtain 2-tert-butoxycarbonylamino-5-hydroxy-pentanoic acid tert-butyl ester as colorless oil (2.9 g, 78.4%). LC-MS (ES) calculated for C₁₄H₂₇NO₅, 289.4; found m/z 290.2 [M+H]⁺.

Triphenylphosphine (5.3 g, 20 mmol) was dissolved in 40 mL of THF. After cooled on ice-water bath, diisopropylazodicarboxylate (5.3 g, 20.0 mmol) in 15 mL THF solution was added from an addition funnel in one portion. The resulted suspension was stirred on ice-water bath for 30 min. 2-tert-Butoxycarbonylamino-5-hydroxy-pentanoic acid tert-butyl ester (2.9 g, 10 mmol) in THF (15 mL) solution was added from the additional funnel and stirred for 30 min. Thioacetic acid (1.59 g, 20.0 mmol) in THF (10 mL) solution was added and the mixture was stirred on ice-water bath for 1 hour. Then it was warmed to room temperature and stirred for another 4 hours to afford the clear pale yellow solution. Ether and hexane were added for extraction and the organic layer was washed with saturated sodium bicarbonate, brine, dried and concentrated. The residue was treated with 10% ether in petroleum ether and the white solid was filtered. The filtrate was concentrated and purified by using an ISCO (400 g) column chromatography, eluting with 2-12% ethyl acetate in hexanes to obtain 5-acetylsulfanyl-2-tert-butoxycarbonylamino-pentanoic acid tert-butyl ester as light yellow oil (3.2 g, 91.9%). LC-MS (ES) calculated for C₁₆H₂₉NO₅S, 347.5; found m/z 348.2 [M+H]⁺.

5-Acetylsulfanyl-2-tert-butoxycarbonylamino-pentanoic acid tert-butyl ester (2.6 g, 7.48 mmol) was suspended in 40 mL of acetic acid and 8 mL of water. Sodium acetate (6.1 g, 74.8 mmol) was added and the mixture was cooled on ice-water bath. Chlorine gas was bubbled into the reaction to afford light yellow color mixture for 10 minutes. Saturated sodium bicarbonate was added to neutralize the mixture and the ethyl acetate was used for extraction. The organic solvents were evaporated to afford 1,1-dioxo-1lambda*6*-[1,2]thiazinane-2,3-dicarboxylic acid di-tert-butyl ester as light brown solid (2.9 g, 100%). ¹H NMR(CHLOROFORM-d) δ 4.73 (d, J=9.3 Hz, 1H), 3.61-3.80 (m, 2H), 1.94-2.25 (m, 4H), 1.49 (s, 9H), 1.44 (s, 9H).

1,1-Dioxo-1lambda*6*-[1,2]thiazinane-2,3-dicarboxylic acid di-tert-butyl ester (1.2 g, 3.576 mmol) was suspended in 50 mL of methylene chloride. After cooled on ice-water bath, 5 mL of TFA was added drop wise and the mixture was stirred at room temperature for 2 hours. After adding 20 mL of toluene, the solvents were evaporated to afford the solid residue. The crude product was purified by using an ISCO (40 g) column chromatography, eluting with 5-30% ethyl acetate in hexanes to obtain 1,1-dioxo-1lambda*6*-[1,2]thiazinane-3-carboxylic acid tert-butyl ester as a fluffy solid (133 mg, 15%). LC-MS (ES) calculated for C₉H₁₇NO₄S, 235.3; found m/z 236.1 [M+H]⁺.

### Part II

### Preparation of Preferred Embodiments of the Invention

### Example 1

### [[3-(4′,5′-Difluoro-2′-methoxy-biphenyl-4-yloxymethyl)-benzyl]-(2,2-dimethyl-propionyl)-amino]-acetic acid

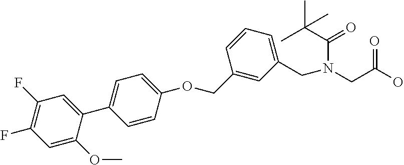

A mixture of [3-(4′,5′-difluoro-2′-methoxy-biphenyl-4-yloxymethyl)-benzylamino]-acetic acid ethyl ester hydrochloride (165 mg, 0.35 mmol), 2,2-dimethyl-propionyl chloride (70 mg, 0.58 mmol) and triethylamine (100 mg, 1.0 mmol) in dichloromethane (10 mL) was stirred at room temperature for 1 h. Solvent was removed under vacuum. The crude solid was diluted with ethyl acetate (50 mL) and washed in sequences with saturated ammonium chloride (50 mL), water (50 mL), sodium hydroxide solution (0.1N, 50 mL), and brine (50 mL). The organic layer was dried with anhydrous sodium sulfate and solvent was removed. The residue was purified through flash column chromatography to yield [[3-(4′,5′-difluoro-2′-methoxy-biphenyl-4-yloxymethyl)-benzyl]-(2,2-dimethyl-propionyl)-amino]-acetic acid ethyl ester intermediate (175 mg, 97% yield).

The intermediate [[3-(4′,5′-difluoro-2′-methoxy-biphenyl-4-yloxymethyl)-benzyl]-(2,2-dimethyl-propionyl)-amino]-acetic acid ethyl ester (175 mg) was treated with lithium hydroxide solution (0.5N, 2 mL) and THF (4 mL). The mixture was stirred for 3 h at room temperature. The reaction mixture was diluted with ethyl acetate (50 mL) and washed with hydrochloric acid (1N, 50 mL). The organic layer was washed with water (50 mL), brine (50 mL) and dried with anhydrous sodium sulfate. After evaporation of solvents, the pure amorphous solid was treated with a 1:1 ratio of acetonitrile and water (10 mL) and the mixture was lyophilized to afford [[3-(4′,5′-difluoro-2′-methoxy-biphenyl-4-yloxymethyl)-benzyl]-(2,2-dimethyl-propionyl)-amino]-acetic acid (160 mg, 98% yield) as a semi solid. LRMS calcd for C₂₈H₂₉F₂NO₅ (m/e) 498.20 (M+H), obsd 498.1 (ES+).

### Example 2

### [[3-(4′,5′-Difluoro-2′-methoxy-biphenyl-4-yloxymethyl)-benzyl]-(3-methyl-butyryl)-amino]-acetic acid

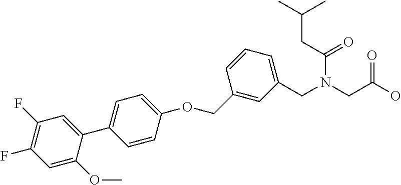

With a method similar to that used for the preparation of [[3-(4′,5′-difluoro-2′-methoxy-biphenyl-4-yloxymethyl)-benzyl]-(2,2-dimethyl-propionyl)-amino]-acetic acid, [[3-(4′,5′-difluoro-2′-methoxy-biphenyl-4-yloxymethyl)-benzyl]-(3-methyl-butyryl)-amino]-acetic acid was prepared from [3-(4′,5′-difluoro-2′-methoxy-biphenyl-4-yloxymethyl)-benzylamino]-acetic acid ethyl ester hydrochloride and 3-methyl-butyryl chloride. LRMS calcd for C₂₈H₂₉F₂NO₅ (m/e) 498.20 (M+H), obsd 498.1 (ES+).

### Example 3

### [[3-(4′,5′-Difluoro-2′-methoxy-biphenyl-4-yloxymethyl)-benzyl]-(morpholine-4-carbonyl)-amino]-acetic acid

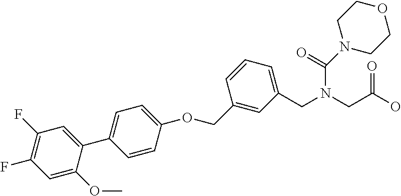

With a method similar to that used for the preparation of [[3-(4′,5′-difluoro-2′-methoxy-biphenyl-4-yloxymethyl)-benzyl]-(2,2-dimethyl-propionyl)-amino]-acetic acid, [[3-(4′,5′-difluoro-2′-methoxy-biphenyl-4-yloxymethyl)-benzyl]-(morpholine-4-carbonyl)-amino]-acetic acid was prepared from [3-(4′,5′-difluoro-2′-methoxy-biphenyl-4-yloxymethyl)-benzylamino]-acetic acid ethyl ester hydrochloride and morpholine-4-carbonyl chloride. LRMS calcd for C₂₈H₂₈F₂N₂O₆ (m/e) 527.19 (M+H), obsd 527.2 (ES+).

### Example 4

### {Cyclobutanecarbonyl-[3-(4′,5′-difluoro-2′-methoxy-biphenyl-4-yloxymethyl)-benzyl]-amino}-acetic acid

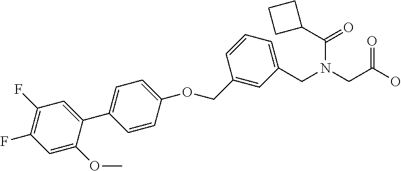

With a method similar to that used for the preparation of [[3-(4′,5′-difluoro-2′-methoxy-biphenyl-4-yloxymethyl)-benzyl]-(2,2-dimethyl-propionyl)-amino]-acetic acid, {cyclobutanecarbonyl-[3-(4′,5′-difluoro-2′-methoxy-biphenyl-4-yloxymethyl)-benzyl]-amino}-acetic acid was prepared from [3-(4′,5′-difluoro-2′-methoxy-biphenyl-4-yloxymethyl)-benzylamino]-acetic acid ethyl ester hydrochloride and cyclobutanecarbonyl chloride. LRMS calcd for C₂₈H₂₇F₂NO₅ (m/e) 496.19 (M+H), obsd 496.2 (ES+).

### Example 5

### {Benzoyl-[3-(4′,5′-difluoro-2′-methoxy-biphenyl-4-yloxymethyl)-benzyl]-amino}-acetic acid

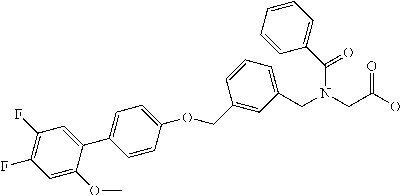

With a method similar to that used for the preparation of [[3-(4′,5′-difluoro-2′-methoxy-biphenyl-4-yloxymethyl)-benzyl]-(2,2-dimethyl-propionyl)-amino]-acetic acid, {benzoyl-[3-(4′,5′-difluoro-2′-methoxy-biphenyl-4-yloxymethyl)-benzyl]-amino}-acetic acid was prepared from [3-(4′,5′-difluoro-2′-methoxy-biphenyl-4-yloxymethyl)-benzylamino]-acetic acid ethyl ester hydrochloride and benzoyl chloride. LRMS calcd for C₃₀H₂₅F₂NO₅ (m/e) 518.17 (M+H), obsd 518.1 (ES+).

### Example 6

### {[3-(4′,5′-Difluoro-2′-methoxy-biphenyl-4-yloxymethyl)-benzyl]-isopropoxycarbonyl-amino}-acetic acid

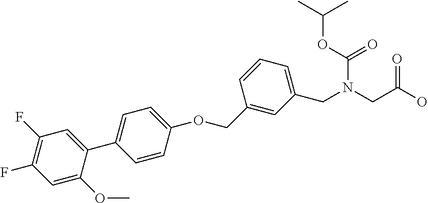

With a method similar to that used for the preparation of [[3-(4′,5′-difluoro-2′-methoxy-biphenyl-4-yloxymethyl)-benzyl]-(2,2-dimethyl-propionyl)-amino]-acetic acid, {[3-(4′,5′-difluoro-2′-methoxy-biphenyl-4-yloxymethyl)-benzyl]-isopropoxycarbonyl-amino}-acetic acid was prepared from [3-(4′,5′-difluoro-2′-methoxy-biphenyl-4-yloxymethyl)-benzylamino]-acetic acid ethyl ester hydrochloride and isopropyl chloroformate. LRMS calcd for C₂₇H₂₇F₂NO₆ (m/e) 500.18 (M+H), obsd 500.1 (ES+).

### Example 7

### {[3-(4′,5′-Difluoro-2′-methoxy-biphenyl-4-yloxymethyl)-benzyl]-ethanesulfonyl-amino}-acetic acid

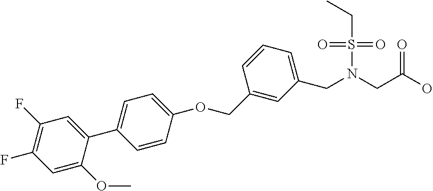

With a method similar to that used for the preparation of [[3-(4′,5′-difluoro-2′-methoxy-biphenyl-4-yloxymethyl)-benzyl]-(2,2-dimethyl-propionyl)-amino]-acetic acid, {[3-(4′,5′-difluoro-2′-methoxy-biphenyl-4-yloxymethyl)-benzyl]-ethanesulfonyl-amino}-acetic acid was prepared from [3-(4′,5′-difluoro-2′-methoxy-biphenyl-4-yloxymethyl)-benzylamino]-acetic acid ethyl ester hydrochloride and ethanesulfonyl chloride. LRMS calcd for C₂₅H₂₅F₂NO₆S (m/e) 504.14 (M−H), obsd 504.1 (ES−).

### Example 8

### {Benzenesulfonyl-[3-(4′,5′-difluoro-2′-methoxy-biphenyl-4-yloxymethyl)-benzyl]-amino}-acetic acid

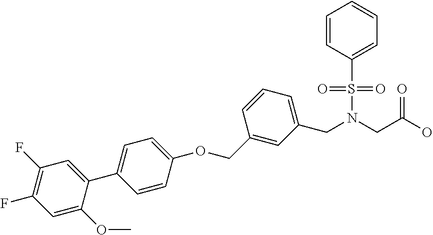

With a method similar to that used for the preparation of [[3-(4′,5′-difluoro-2′-methoxy-biphenyl-4-yloxymethyl)-benzyl]-(2,2-dimethyl-propionyl)-amino]-acetic acid, {benzenesulfonyl-[3-(4′,5′-difluoro-2′-methoxy-biphenyl-4-yloxymethyl)-benzyl]-amino}-acetic acid was prepared from [3-(4′,5′-difluoro-2′-methoxy-biphenyl-4-yloxymethyl)-benzylamino]-acetic acid ethyl ester hydrochloride and benzenesulfonyl chloride. LRMS calcd for C₂₉H₂₅F₂NO₆S (m/e) 552.14 (M−H), obsd 552.1 (ES−).

### Example 9

### {Cyclopropanesulfonyl-[3-(4′,5′-difluoro-2′-methoxy-biphenyl-4-yloxymethyl)-benzyl]-amino}-acetic acid

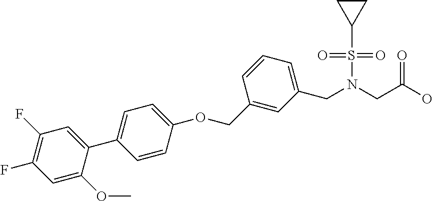

With a method similar to that used for the preparation of [[3-(4′,5′-difluoro-2′-methoxy-biphenyl-4-yloxymethyl)-benzyl]-(2,2-dimethyl-propionyl)-amino]-acetic acid, {cyclopropanesulfonyl-[3-(4′,5′-difluoro-2′-methoxy-biphenyl-4-yloxymethyl)-benzyl]-amino}-acetic acid was prepared from [3-(4′,5′-difluoro-2′-methoxy-biphenyl-4-yloxymethyl)-benzylamino]-acetic acid ethyl ester hydrochloride and cyclopropane sulfonyl chloride. LRMS calcd for C₂₆H₂₅F₂NO₆S (m/e) 516.14 (M−H), obsd 516.0 (ES−).

### Example 10

### {[3-(4′,5′-Difluoro-2′-methoxy-biphenyl-4-yloxymethyl)-benzyl]-methanesulfonyl-amino}-acetic acid

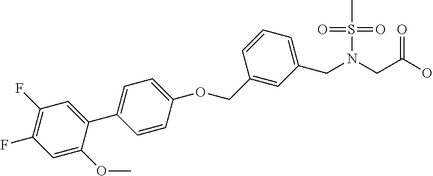

With a method similar to that used for the preparation of [[3-(4′,5′-difluoro-2′-methoxy-biphenyl-4-yloxymethyl)-benzyl]-(2,2-dimethyl-propionyl)-amino]-acetic acid, {[3-(4′,5′-difluoro-2′-methoxy-biphenyl-4-yloxymethyl)-benzyl]-methanesulfonyl-amino}-acetic acid was prepared from [3-(4′,5′-difluoro-2′-methoxy-biphenyl-4-yloxymethyl)-benzylamino]-acetic acid ethyl ester hydrochloride and methanesulfonyl chloride. LRMS calcd for C₂₄H₂₃F₂NO₆S (m/e) 490.12 (M−H), obsd 489.9 (ES−).

### Example 11

### [[3-(2′,4′-Difluoro-biphenyl-4-yloxymethyl)-benzyl]-(2,2-dimethyl-propionyl)-amino]-acetic acid

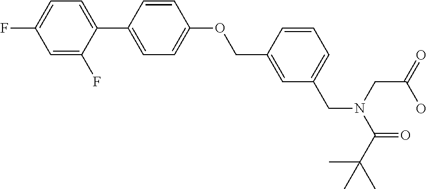

[3-(2′,4′-Difluoro-biphenyl-4-yloxymethyl)-benzylamino]-acetic acid ethyl ester hydrochloride (150 mg, 0.335 mmol) was suspended in methylene chloride (5 mL) and triethylamine (0.1 mL) was added. The clear solution was cooled in ice bath and trimethylacetyl chloride (42 mg, 0.348 mmol) was added. The solution was stirred at room temperature for 5 hrs and solvents were evaporated. The residue was extracted with ethyl acetate and 0.1N hydrochloric acid. The organic layer was washed with water and concentrated sodium bicarbonate solution. After the evaporation of solvents, an oily material was obtained. This oily material was dissolved in THF (4 mL), methanol (0.5 mL) and lithium hydroxide solution (0.5N, 2 mL) was added. The mixture was stirred at room temperature for 2 hrs. The mixture was evaporated and the residue was extracted with ethyl acetate and dilute hydrochloric acid. The organic layer was washed with water and brine, dried over sodium sulfate and solvent was evaporated to give a waxy material as [[3-(2′,4′-difluoro-biphenyl-4-yloxymethyl)-benzyl]-(2,2-dimethyl-propionyl)-amino]-acetic acid (156 mg, 100% yield). HRMS calcd for C₂₇H₂₇NO₄F₂ (m/e) 468.1981 (M+H), obsd 468.1979 (ES+); ¹H-NMR (300 MHz, DMSO-d₆) δ ppm 1.20 (s, 9H), 3.52-5.02 (m, 4H), 5.16 (s, 2H), 7.10 (d, J=8.8 Hz, 2H), 7.12-7.23 (m, 2H), 7.26-7.41 (m, 4H), 7.44 (d, J=7.5 Hz, 2H), 7.53 (td, J=8.8, 7.2 Hz, 1H), 12.60 (br s, 1H).

### Example 12

### {Cyclopropanecarbonyl-[3-(2′,4′-difluoro-biphenyl-4-yloxymethyl)-benzyl]-amino}-acetic acid

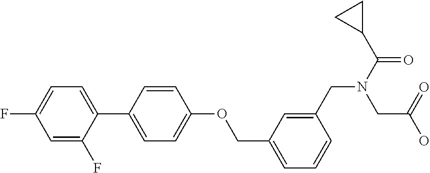

This compound was prepared with the same method as described for the preparation of [[3-(2′,4′-difluoro-biphenyl-4-yloxymethyl)-benzyl]-(2,2-dimethyl-propionyl)-amino]-acetic acid. {Cyclopropanecarbonyl-[3-(2′,4′-difluoro-biphenyl-4-yloxymethyl)-benzyl]-amino}-acetic acid was prepared from [3-(2′,4′-difluoro-biphenyl-4-yloxymethyl)-benzylamino]-acetic acid ethyl ester hydrochloride and cyclopropanecarbonyl chloride. HRMS calcd for C₂₆H₂₃NO₄F₂ (m/e) 452.1668 (M+H), obsd 452.1667 (ES+).

### Example 13

### [[3-(2′,4′-Difluoro-biphenyl-4-yloxymethyl)-benzyl]-(morpholine-4-carbonyl)-amino]-acetic acid

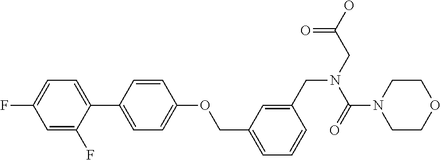

With the same method as described for the preparation of [[3-(2′,4′-difluoro-biphenyl-4-yloxymethyl)-benzyl]-(2,2-dimethyl-propionyl)-amino]-acetic acid, [[3-(2′,4′-difluoro-biphenyl-4-yloxymethyl)-benzyl]-(morpholine-4-carbonyl)-amino]-acetic acid was prepared from [3-(2′,4′-difluoro-biphenyl-4-yloxymethyl)-benzylamino]-acetic acid ethyl ester hydrochloride and morpholine-4-carbonyl chloride. LRMS calcd for C₂₇H₂₆F₂N₂O₅ (m/e) 497.18 (M+H), obsd 497.1 (ES+).

### Example 14

### [[3-(2′,4′-Difluoro-biphenyl-4-yloxymethyl)-benzyl]-(1-methyl-imidazole-2-carbonyl)-amino]-acetic acid

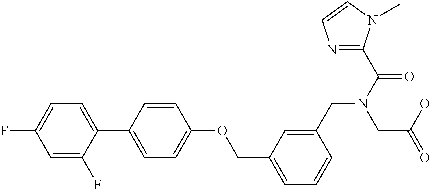

[3-(2′,4′-Difluoro-biphenyl-4-yloxymethyl)-benzylamino]-acetic acid ethyl ester (205 mg, 0.5 mmol) was mixed with N-methyl-imidazole-2-carboxylic acid (126 mg, 1.0 mmol) in DMF (6 mL). The mixture was stirred and BOP reagent (331.8 mg, 0.749 mmol), diisopropylethylamine (0.18 mL, 0.97 mmol) was added. The mixture was stirred at room temperature overnight and solvent was evaporated. The residue was extracted with ethyl acetate and saturated ammonium chloride solution. The organic layer was washed with water and concentrated sodium bicarbonate solution. Solvent was removed and the residue was purified through a flash column chromatography (ethyl acetate in hexanes 10% to 100%) to give [[3-(2′,4′-difluoro-biphenyl-4-yloxymethyl)-benzyl]-(1-methyl-imidazole-2-carbonyl)-amino]-acetic acid ethyl ester (237 mg, 91.5% yield).

[[3-(2′,4′-Difluoro-biphenyl-4-yloxymethyl)-benzyl]-(1-methyl-imidazole-2-carbonyl)-amino]-acetic acid ethyl ester (237 mg, 0.46 mmol) was dissolved in THF (4 mL) and lithium hydroxide solution (0.5N, 1 mL) was added. The mixture was stirred at room temperature for 4 hrs and solvent was evaporated. The residue was treated with hydrochloric acid (1N, 0.5 mL). The white precipitate was filtered and dried to give [[3-(2′,4′-difluoro-biphenyl-4-yloxymethyl)-benzyl]-(1-methyl-imidazole-2-carbonyl)-amino]-acetic acid (224 mg, 100% yield). HRMS calcd for C₂₇H₂₃N₃O₄F₂ (m/e) 492.1730 (M+H), obsd 492.1726.

### Example 15

### [[3-(2′,4′-Difluoro-biphenyl-4-yloxymethyl)-benzyl]-(pyridine-3-carbonyl)-amino]-acetic acid

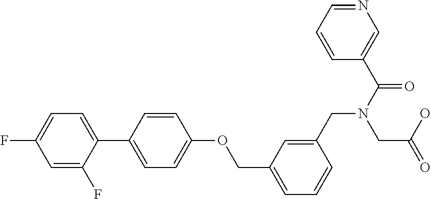

With the same method as described for the preparation of [[3-(2′,4′-difluoro-biphenyl-4-yloxymethyl)-benzyl]-(1-methyl-imidazole-2-carbonyl)-amino]-acetic acid, [[3-(2′,4′-difluoro-biphenyl-4-yloxymethyl)-benzyl]-(pyridine-3-carbonyl)-amino]-acetic acid was prepared from [3-(2′,4′-difluoro-biphenyl-4-yloxymethyl)-benzylamino]-acetic acid ethyl ester and pyridine-3-carboxylic acid. HRMS calcd for C₂₈H₂₂N₂O₄F₂ (m/e) 489.1621 (M+H), obsd 489.1619 (ES+).

### Example 16

### [[3-(2′,4′-Difluoro-biphenyl-4-yloxymethyl)-benzyl]-(pyridine-2-carbonyl)-amino]-acetic acid

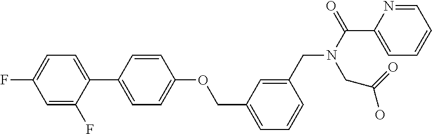

With the same method as described for the preparation of [[3-(2′,4′-difluoro-biphenyl-4-yloxymethyl)-benzyl]-(1-methyl-imidazole-2-carbonyl)-amino]-acetic acid, [[3-(2′,4′-difluoro-biphenyl-4-yloxymethyl)-benzyl]-(pyridine-2-carbonyl)-amino]-acetic acid was prepared from [3-(2′,4′-difluoro-biphenyl-4-yloxymethyl)-benzylamino]-acetic acid ethyl ester and pyridine-2-carboxylic acid. HRMS calcd for C₂₈H₂₂N₂O₄F₂ (m/e) 489.1621 (M+H), obsd 489.1619 (ES+); ¹H-NMR (300 MHz, DMSO-d₆) δ ppm 12.50 (br s, 1H), 8.55 (br s, 1H), 7.86-7.98 (m, 1H), 7.06-7.73 (m, 13H), 5.13-5.16 (2×s, 2H), 4.70-4.75 (2×s, 2H), 3.99-4.21 (2×s, 2H).

### Example 17

### [[3-(2′,4′-Difluoro-biphenyl-4-yloxymethyl)-benzyl]-(3-methoxy-2-methyl-propionyl)-amino]-acetic acid

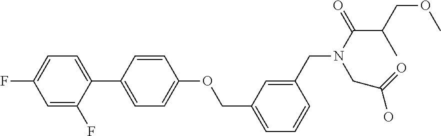

With the same method as described for the preparation of [[3-(2′,4′-difluoro-biphenyl-4-yloxymethyl)-benzyl]-(1-methyl-imidazole-2-carbonyl)-amino]-acetic acid, [[3-(2′,4′-fluoro-biphenyl-4-yloxymethyl)-benzyl]-(3-methoxy-2-methyl-propionyl)-amino]-acetic acid was prepared from [3-(2′,4′-difluoro-biphenyl-4-yloxymethyl)-benzylamino]-acetic acid ethyl ester and 3-methoxy-2-methylpropionic acid. HRMS calcd for C₂₇H₂₇NO₅F₂ (m/e) 484.1930 (M+H), obsd 484.1929 (ES+).

### Example 18

### {Ethanesulfonyl-[3-(2′,4′,5′-trifluoro-biphenyl-4-yloxymethyl)-benzyl]-amino}-acetic acid

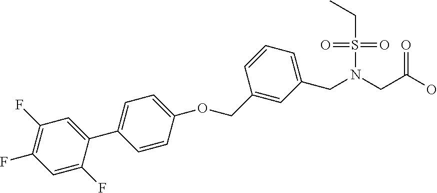

To a DMF (15 mL) solution containing ethanesulfonylaminoacetic acid ethyl ester (292.5 mg, 1.5 mmol) and 4′-(3-bromomethyl-benzyloxy)-2,4,5-trifluoro-biphenyl (610.5 mg, 1.5 mmol) was added potassium carbonate (414 mg, 3 mmol). The mixture was stirred at room temperature for 4 hrs and solvent was evaporated. The residue was extracted with ethyl acetate and water. The organic layer was washed with brine and solvent was evaporated. The crude mixture was purified through a flash column chromatography (50 g silica gel, ethyl acetate in hexanes 0% to 40% in 25 minutes) to give {ethanesulfonyl-[3-(2′,4′,5′-trifluoro-biphenyl-4-yloxymethyl)-benzyl]-amino}-acetic acid ethyl ester as a white solid (680 mg, 87% yield). LRMS calcd for C₂₆H₂₆F₃NO₅S (m/e) 520.15 (M−H), obsd 520.0 (ES−).

{Ethanesulfonyl-[3-(2′,4′,5′-trifluoro-biphenyl-4-yloxymethyl)-benzyl]-amino}-acetic acid ethyl ester (660 mg, 1.27 mmol) was dissolved in THF (9 mL) and lithium hydroxide solution (0.5N, 3.5 mL) was added. The mixture was stirred at 0° C. for 45 minutes and at room temperature for 1 hr. Solvent was removed and the white solid was dissolved in warm water (50 mL). Hydrochloric acid (1N, 3 mL) was added and the precipitate was filtered and dried to give {ethanesulfonyl-[3-(2′,4′,5′-trifluoro-biphenyl-4-yloxymethyl)-benzyl]-amino}-acetic acid (618 mg, 99% yield). LRMS calcd for C₂₄H₂₂F₃NO₅S (m/e) 492.12 (M−H), obsd 492.1.

### Example 19

### {[3-(4′,5′-Difluoro-2′-methoxy-biphenyl-4-yloxymethyl)-benzyl]-methoxycarbonyl-amino}-acetic acid

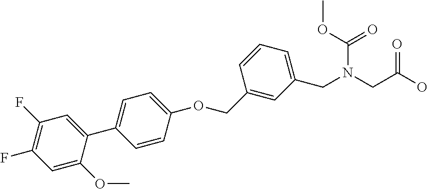

A mixture of [3-(4′,5′-difluoro-2′-methoxy-biphenyl-4-yloxymethyl)-benzylamino]-acetic acid tert-butyl ester (200 mg, 0.43 mmol), methyl chloroformate (100 mg, 1.06 mmol), triethylamine (200 mg, 2.0 mmol), in dichloromethane (10 mL) was stirred at room temperature for 3 h. Dichloromethane was removed under vacuum. The crude solid was diluted with ethyl acetate (50 mL) and washed with saturated ammonium chloride (50 mL), water (50 mL) and brine (50 mL). The organic layer was dried with anhydrous sodium sulfate and the solvent was removed to yield {[3-(4′,5′-difluoro-2′-methoxy-biphenyl-4-yloxymethyl)-benzyl]-methoxycarbonyl-amino}-acetic acid tert-butyl ester (200 mg, 88% yield).

{[3-(4′,5′-Difluoro-2′-methoxy-biphenyl-4-yloxymethyl)-benzyl]-methoxycarbonyl-amino}-acetic acid tert-butyl ester (200 mg, 0.38 mmol) was treated with hydrogen chloride in dioxane (4.0N, 6 mL) and the reaction mixture was stirred for 3 h. The reaction mixture was diluted with ethyl acetate (50 mL) and washed with water (50 mL), and brine (50 mL). The organic layer was dried with anhydrous sodium sulfate and the solvent was removed. The amorphous solid was treated with a 1:1 ratio of acetonitrile and water (10 mL) and the mixture was lyophilized to afford {[3-(4′,5′-difluoro-2′-methoxy-biphenyl-4-yloxymethyl)-benzyl]-methoxy carbonyl-amino}-acetic acid (174 mg, 98% yield) as a semi solid. LRMS calcd for C₂₅H₂₃F₂NO₆ (m/e) 472.15 (M+H), obsd 472.1 (ES+).

### Example 20

### {[3-(4′,5′-Difluoro-2′-methoxy-biphenyl-4-yloxymethyl)-benzyl]-ethoxycarbonyl-amino}-acetic acid

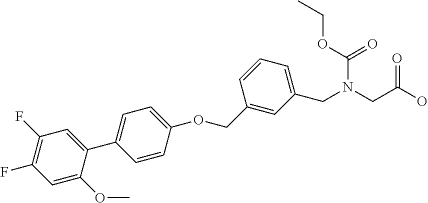

With a method similar to that used for the preparation of {[3-(4′,5′-difluoro-2′-methoxy-biphenyl-4-yloxymethyl)-benzyl]-methoxy carbonyl-amino}-acetic acid, {[3-(4′,5′-difluoro-2′-methoxy-biphenyl-4-yloxymethyl)-benzyl]-ethoxycarbonyl-amino}-acetic acid was prepared from [3-(4′,5′-difluoro-2′-methoxy-biphenyl-4-yloxymethyl)-benzylamino]-acetic acid tert-butyl ester and ethyl chloroformate. LRMS calcd for C₂₆H₂₅F₂NO₆ (m/e) 486.16 (M+H), obsd 486.1 (ES+).

### Example 21

### {Acetyl-[3-(4′,5′-difluoro-2′-methoxy-biphenyl-4-yloxymethyl)-benzyl]-amino}-acetic acid

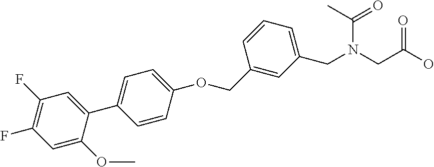

With a method similar to that used for the preparation of {[3-(4′,5′-difluoro-2′-methoxy-biphenyl-4-yloxymethyl)-benzyl]-methoxy carbonyl-amino}-acetic acid, {acetyl-[3-(4′,5′-difluoro-2′-methoxy-biphenyl-4-yloxymethyl)-benzyl]-amino}-acetic acid was prepared from [3-(4′,5′-difluoro-2′-methoxy-biphenyl-4-yloxymethyl)-benzylamino]-acetic acid tert-butyl ester and acetic anhydride. LRMS calcd for C₂₅H₂₃F₂NO₅ (m/e) 456.15 (M+H), obsd 456.0 (ES+).

### Example 22

### {Cyclopropanecarbonyl-[3-(4′,5′-difluoro-2′-methoxy-biphenyl-4-yloxymethyl)-benzyl]-amino}-acetic acid

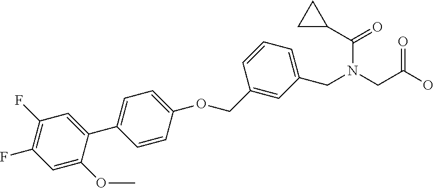

With a method similar to that used for the preparation of {[3-(4′,5′-difluoro-2′-methoxy-biphenyl-4-yloxymethyl)-benzyl]-methoxy carbonyl-amino}-acetic acid, {cyclopropanecarbonyl-[3-(4′,5′-difluoro-2′-methoxy-biphenyl-4-yloxymethyl)-benzyl]-amino}-acetic acid was prepared from [3-(4′,5′-difluoro-2′-methoxy-biphenyl-4-yloxymethyl)-benzylamino]-acetic acid tert-butyl ester and cyclopropanecarbonyl chloride. LRMS calcd for C₂₇H₂₅F₂NO₅ (m/e) 482.17 (M+H), obsd 482.1 (ES+).

### Example 23

### {[3-(4′,5′-Difluoro-2′-methoxy-biphenyl-4-yloxymethyl)-benzyl]-propionyl-amino}-acetic acid

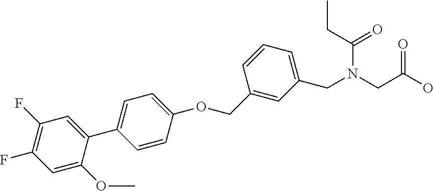

With a method similar to that used for the preparation of {[3-(4′,5′-difluoro-2′-methoxy-biphenyl-4-yloxymethyl)-benzyl]-methoxy carbonyl-amino}-acetic acid, {[3-(4′,5′-difluoro-2′-methoxy-biphenyl-4-yloxymethyl)-benzyl]-propionyl-amino}-acetic acid was prepared from [3-(4′,5′-difluoro-2′-methoxy-biphenyl-4-yloxymethyl)-benzylamino]-acetic acid tert-butyl ester and propionic anhydride. LRMS calcd for C₂₆H₂₅F₂NO₅ (m/e) 470.17 (M+H), obsd 470.1 (ES+).

### Example 24

### {(2-Cyclopropyl-acetyl)-[3-(4′,5′-difluoro-2′-methoxy-biphenyl-4-yloxymethyl)-benzyl]-amino}-acetic acid

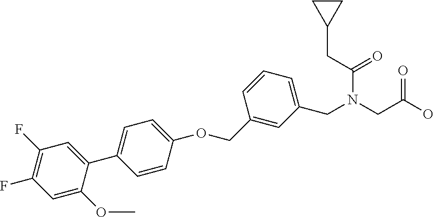

A mixture of [3-(4′,5′-difluoro-2′-methoxy-biphenyl-4-yloxymethyl)-benzylamino]-acetic acid ethyl ester hydrochloride (110 mg, 0.23 mmol), cyclopropyl-acetic acid (80 mg, 0.8 mmol), BOP reagent (330 mg, 0.75 mmol) and triethylamine (200 mg, 2.0 mmol), in dichloromethane (10 mL) was stirred at room temperature overnight. The mixture was concentrated and diluted with ethyl acetate (50 mL) and saturated ammonium chloride (50 mL). The organic layer was washed with water and brine, dried with anhydrous sodium sulfate and the solvent was removed. The residue was purified on a flash chromatography column with ethyl acetate in hexanes to yield {(2-cyclopropyl-acetyl)-[3-(4′,5′-difluoro-2′-methoxy-biphenyl-4-yloxymethyl)-benzyl]-amino}-acetic acid ethyl ester (120 mg, 100% yield).

{(2-cyclopropyl-acetyl)-[3-(4′,5′-difluoro-2′-methoxy-biphenyl-4-yloxymethyl)-benzyl]-amino}-acetic acid ethyl ester (120 mg, 2.3 mmol) was treated with lithium hydroxide solution (0.5N, 2 mL) and THF (4 mL). The reaction mixture was stirred for 3 h and diluted with ethyl acetate (50 mL) and hydrochloric acid (0.1N, 50 mL). The organic layer was washed with water and brine and dried with anhydrous sodium sulfate. The solvent was removed and the residue was treated with a 1:1 ratio of acetonitrile and water (10 mL) and the mixture was lyophilized to afford {(2-cyclopropyl-acetyl)-[3-(4′,5′-difluoro-2′-methoxy-biphenyl-4-yloxymethyl)-benzyl]-amino}-acetic acid (90 mg, 80% yield) as a semi solid. LRMS calcd for C₂₈H₂₇F₂NO₅ (m/e) 496.19 (M+H), obsd 496.1 (ES+).

### Example 25

### [[3-(4′,5′-Difluoro-2′-methoxy-biphenyl-4-yloxymethyl)-benzyl]-((S)-tetrahydro-furan-2-carbonyl)-amino]-acetic acid

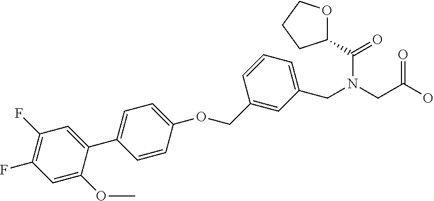

With a method similar to that used for the preparation of {(2-cyclopropyl-acetyl)-[3-(4′,5′-difluoro-2′-methoxy-biphenyl-4-yloxymethyl)-benzyl]-amino}-acetic acid, [[3-(4′,5′-difluoro-2′-methoxy-biphenyl-4-yloxymethyl)-benzyl]-((S)-tetrahydro-furan-2-carbonyl)-amino]-acetic acid was prepared from [3-(4′,5′-difluoro-2′-methoxy-biphenyl-4-yloxymethyl)-benzylamino]-acetic acid ethyl ester hydrochloride and (S)-tetrahydro-furan-2-carboxylic acid. LRMS calcd for C₂₈H₂₇F₂NO₆ (m/e) 512.18 (M+H), obsd 512.1 (ES+).

### Example 26

### [[3-(4′,5′-Difluoro-2′-methoxy-biphenyl-4-yloxymethyl)-benzyl]-(3-methoxy-propionyl)-amino]-acetic acid

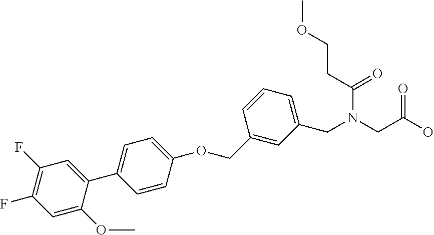

With a method similar to that used for the preparation of {(2-cyclopropyl-acetyl)-[3-(4′,5′-difluoro-2′-methoxy-biphenyl-4-yloxymethyl)-benzyl]-amino}-acetic acid, [[3-(4′,5′-difluoro-2′-methoxy-biphenyl-4-yloxymethyl)-benzyl]-(3-methoxy-propionyl)-amino]-acetic acid was prepared from [3-(4′,5′-difluoro-2′-methoxy-biphenyl-4-yloxymethyl)-benzylamino]-acetic acid ethyl ester hydrochloride and 3-methoxy-propionic acid. LRMS calcd for C₂₇H₂₇F₂NO₆ (m/e) 500.18 (M+H), obsd 500.1 (ES+).

### Example 27

### [[3-(4′,5′-Difluoro-2′-methoxy-biphenyl-4-yloxymethyl)-benzyl]-(thiazole-4-carbonyl)-amino]-acetic acid

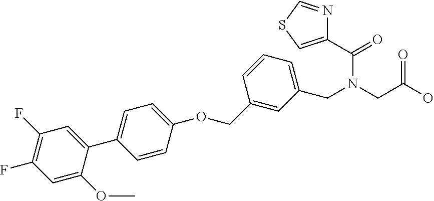

With a method similar to that used for the preparation of {(2-cyclopropyl-acetyl)-[3-(4′,5′-difluoro-2′-methoxy-biphenyl-4-yloxymethyl)-benzyl]-amino}-acetic acid, [[3-(4′,5′-difluoro-2′-methoxy-biphenyl-4-yloxymethyl)-benzyl]-(thiazole-4-carbonyl)-amino]-acetic acid was prepared from [3-(4′,5′-difluoro-2′-methoxy-biphenyl-4-yloxymethyl)-benzylamino]-acetic acid ethyl ester hydrochloride and thiazole-4-carboxylic acid. LRMS calcd for C₂₇H₂₂F₂N₂O₅S (m/e) 525.12 (M+H), obsd 525.0 (ES+).

### Example 28

### [[3-(4′,5′-Difluoro-2′-methoxy-biphenyl-4-yloxymethyl)-benzyl]-(2-methoxy-acetyl)-amino]-acetic acid

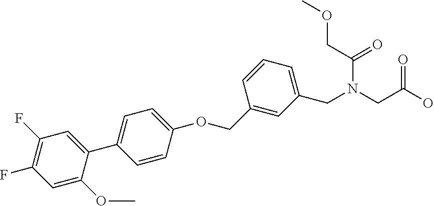

With a method similar to that used for the preparation of {(2-cyclopropyl-acetyl)-[3-(4′,5′-difluoro-2′-methoxy-biphenyl-4-yloxymethyl)-benzyl]-amino}-acetic acid, [[3-(4′,5′-difluoro-2′-methoxy-biphenyl-4-yloxymethyl)-benzyl]-(2-methoxy-acetyl)-amino]-acetic acid was prepared from [3-(4′,5′-difluoro-2′-methoxy-biphenyl-4-yloxymethyl)-benzylamino]-acetic acid ethyl ester hydrochloride and methoxy-acetic acid. LRMS calcd for C₂₆H₂₅F₂NO₆ (m/e) 486.16 (M+H), obsd 486.0 (ES+).

### Example 29

### [[3-(4′,5′-Difluoro-2′-methoxy-biphenyl-4-yloxymethyl)-benzyl]-(1-methyl-1H-imidazole-4-carbonyl)-amino]-acetic acid

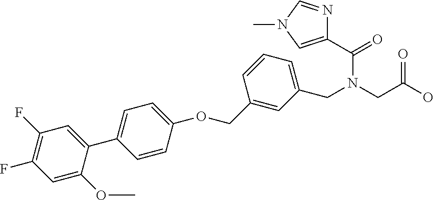

With a method similar to that used for the preparation of {(2-cyclopropyl-acetyl)-[3-(4′,5′-difluoro-2′-methoxy-biphenyl-4-yloxymethyl)-benzyl]-amino}-acetic acid, [[3-(4′,5′-difluoro-2′-methoxy-biphenyl-4-yloxymethyl)-benzyl]-(1-methyl-1H-imidazole-4-carbonyl)-amino]-acetic acid was prepared from [3-(4′,5′-difluoro-2′-methoxy-biphenyl-4-yloxymethyl)-benzylamino]-acetic acid ethyl ester hydrochloride and 1-methyl-1H-imidazole-4-carboxylic acid. LRMS calcd for C₂₈H₂₅F₂N₃O₅ (m/e) 522.18 (M+H), obsd 522.0 (ES+).

### Example 30

### [[3-(4′,5′-difluoro-2′-methoxy-biphenyl-4-yloxymethyl)-benzyl]-(pyridine-3-carbonyl)-amino]-acetic acid

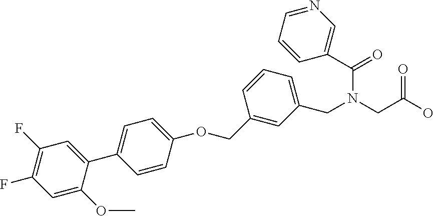

With a method similar to that used for the preparation of {(2-cyclopropyl-acetyl)-[3-(4′,5′-difluoro-2′-methoxy-biphenyl-4-yloxymethyl)-benzyl]-amino}-acetic acid, [[3-(4′,5′-difluoro-2′-methoxy-biphenyl-4-yloxymethyl)-benzyl]-(pyridine-3-carbonyl)-amino]-acetic acid was prepared from [3-(4′,5′-difluoro-2′-methoxy-biphenyl-4-yloxymethyl)-benzylamino]-acetic acid ethyl ester hydrochloride and nicotinic acid. LRMS calcd for C₂₉H₂₄F₂N₂O₅ (m/e) 519.17 (M+H), obsd 519.0 (ES+).

### Example 31

### [3-(4′,5′-Difluoro-2′-methoxy-biphenyl-4-yloxymethyl)-benzyl]-(3-methoxy-2-methyl-propionyl)-amino]-acetic acid

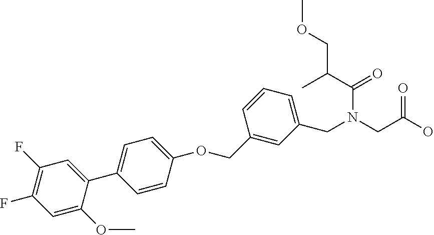

With a method similar to that used for the preparation of {(2-cyclopropyl-acetyl)-[3-(4′,5′-difluoro-2′-methoxy-biphenyl-4-yloxymethyl)-benzyl]-amino}-acetic acid, [3-(4′,5′-difluoro-2′-methoxy-biphenyl-4-yloxymethyl)-benzyl]-(3-methoxy-2-methyl-propionyl)-amino]-acetic acid was prepared from [3-(4′,5′-difluoro-2′-methoxy-biphenyl-4-yloxymethyl)-benzylamino]-acetic acid ethyl ester hydrochloride and 3-methoxy-2-methyl-propionic acid. LRMS calcd for C₂₈H₂₉F₂NO₆ (m/e) 514.20 (M+H), obsd 514.2 (ES+).

### Example 32

### [[3-(4′,5′-Difluoro-2′-methoxy-biphenyl-4-yloxymethyl)-benzyl]-(1-methyl-piperidine-4-carbonyl)-amino]-acetic acid

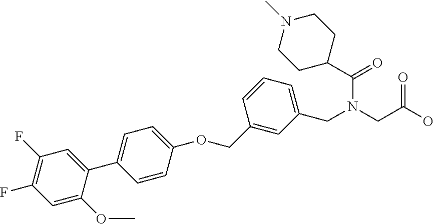

With a method similar to that used for the preparation of {(2-cyclopropyl-acetyl)-[3-(4′,5′-difluoro-2′-methoxy-biphenyl-4-yloxymethyl)-benzyl]-amino}-acetic acid, [[3-(4′,5′-difluoro-2′-methoxy-biphenyl-4-yloxymethyl)-benzyl]-(1-methyl-piperidine-4-carbonyl)-amino]-acetic acid was prepared from [3-(4′,5′-difluoro-2′-methoxy-biphenyl-4-yloxymethyl)-benzylamino]-acetic acid ethyl ester hydrochloride and 1-methyl-piperidine-4-carboxylic acid. LRMS calcd for C₃₀H₃₂F₂N₂O₅ (m/e) 539.23 (M+H), obsd 539.2 (ES+).

### Example 33

### [[3-(4′,5′-Difluoro-2′-methoxy-biphenyl-4-yloxymethyl)-benzyl]-((R)-tetrahydro-furan-2-carbonyl)-amino]-acetic acid

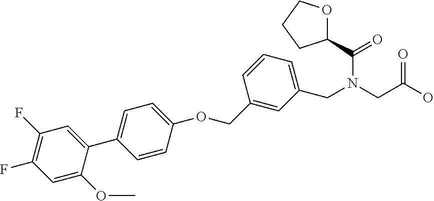

With a method similar to that used for the preparation of {(2-cyclopropyl-acetyl)-[3-(4′,5′-difluoro-2′-methoxy-biphenyl-4-yloxymethyl)-benzyl]-amino}-acetic acid, [[3-(4′,5′-difluoro-2′-methoxy-biphenyl-4-yloxymethyl)-benzyl]-((R)-tetrahydro-furan-2-carbonyl)-amino]-acetic acid was prepared from [3-(4′,5′-difluoro-2′-methoxy-biphenyl-4-yloxymethyl)-benzylamino]-acetic acid ethyl ester hydrochloride and (R)-tetrahydro-furan-2-carboxylic acid. LRMS calcd for C₂₈H₂₇F₂NO₆ (m/e) 512.18 (M+H), obsd 512.1 (ES+).

### Example 34

### [[3-(4′,5′-Difluoro-2′-methoxy-biphenyl-4-yloxymethyl)-benzyl]-(tetrahydro-furan-3-carbonyl)-amino]-acetic acid

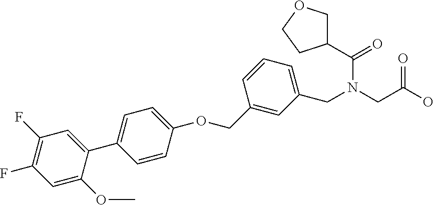

With a method similar to that used for the preparation of {(2-cyclopropyl-acetyl)-[3-(4′,5′-difluoro-2′-methoxy-biphenyl-4-yloxymethyl)-benzyl]-amino}-acetic acid, [[3-(4′,5′-Difluoro-2′-methoxy-biphenyl-4-yloxymethyl)-benzyl]-(tetrahydro-furan-3-carbonyl)-amino]-acetic acid was prepared from [3-(4′,5′-difluoro-2′-methoxy-biphenyl-4-yloxymethyl)-benzylamino]-acetic acid ethyl ester hydrochloride and tetrahydro-furan-3-carboxylic acid. LRMS calcd for C₂₈H₂₇F₂NO₆ (m/e) 512.18 (M+H), obsd 512.1 (ES+).

### Example 35

### {Benzyl-[3-(4′,5′-difluoro-2′-methoxy-biphenyl-4-yloxymethyl)-benzyl]-amino}-acetic acid

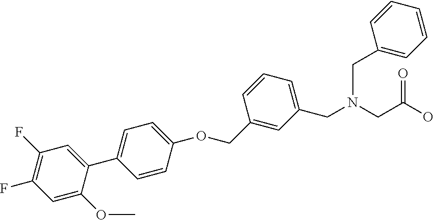

A mixture of [3-(4′,5′-difluoro-2′-methoxy-biphenyl-4-yloxymethyl)-benzylamino]-acetic acid ethyl ester hydrochloride (139 mg, 0.29 mmol), benzyl bromide (51 mg, 0.3 mmol), and potassium carbonate (100 mg, 0.73 mmol) in THF (10 mL) was stirred at 75° C. for 3 h. The mixture was diluted with ethyl acetate (50 mL) and washed with water (50 mL) and brine (50 mL). The organic layer was dried with anhydrous sodium sulfate and the solvent was removed. The residue was purified on a flash chromatography column with ethyl acetate in hexanes to yield {benzyl-[3-(4′,5′-difluoro-2′-methoxy-biphenyl-4-yloxymethyl)-benzyl]-amino}-acetic acid ethyl ester (80 mg, 52% yield).

{Benzyl-[3-(4′,5′-difluoro-2′-methoxy-biphenyl-4-yloxymethyl)-benzyl]-amino}-acetic acid ethyl ester (80 mg, 0.15 mmol) was treated with lithium hydroxide solution (0.5N, 2 mL) and THF (4 mL). The reaction mixture was stirred for 3 h and hydrochloric acid (1N, 1.2 mL) was added. The mixture was diluted with ethyl acetate (50 mL) and washed with water and brine. The organic layer was dried with anhydrous sodium sulfate and the solvent was removed. The residue was treated with a 1:1 ratio of acetonitrile and water (10 mL) and the mixture was lyophilized to afford {benzyl-[3-(4′,5′-difluoro-2′-methoxy-biphenyl-4-yloxymethyl)-benzyl]-amino}-acetic acid (68 mg, 89% yield). LRMS calcd for C₃₀H₂₇F₂NO₄ (m/e) 504.19 (M+H), obsd 504.2 (ES+).

### Example 36

### {[3-(4′,5′-Difluoro-2′-methoxy-biphenyl-4-yloxymethyl)-benzyl]-ethyl-amino}-acetic acid

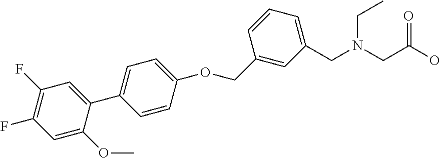

A mixture of [3-(4′,5′-difluoro-2′-methoxy-biphenyl-4-yloxymethyl)-benzylamino]-acetic acid ethyl ester hydrochloride (100 mg, 0.21 mmol), iodoethane (100 mg, 0.7 mmol), and triethylamine (100 mg, 1.0 mmol), in dichloromethane (10 mL) was stirred at room temperature for 3 h. Solvent was removed and the mixture was diluted with ethyl acetate (50 mL) and saturated ammonium chloride (50 mL). The organic layer was washed with water and brine, dried with anhydrous sodium sulfate and the solvent was removed. The residue was purified on a flash chromatography column with ethyl acetate in hexanes to yield {[3-(4′,5′-difluoro-2′-methoxy-biphenyl-4-yloxymethyl)-benzyl]-ethyl-amino}-acetic acid ethyl ester (50 mg, 54% yield).

{[3-(4′,5′-difluoro-2′-methoxy-biphenyl-4-yloxymethyl)-benzyl]-ethyl-amino}-acetic acid ethyl ester (50 mg, 0.11 mmol) was treated with lithium hydroxide solution (0.5N, 2 mL) and THF (4 mL) and the reaction mixture was stirred for 3 h. The reaction mixture was treated with hydrochloric acid (1N, 1.2 mL) and diluted with ethyl acetate (50 mL). The mixture was washed with water and brine and the organic layer was dried with sodium sulfate. Solvent was removed and the residue was treated with a 1:1 ratio of acetonitrile and water (10 mL) and the mixture was lyophilized to afford {[3-(4′,5′-difluoro-2′-methoxy-biphenyl-4-yloxymethyl)-benzyl]-ethyl-amino}-acetic acid (47 mg, 100% yield). LRMS calcd for C₂₅H₂₅F₂NO₄ (m/e) 442.18 (M+H), obsd 442.1 (ES+).

### Example 37

### [3-(4′,5′-Difluoro-2′-methoxy-biphenyl-4-yloxymethyl)-benzylamino]-acetic acid

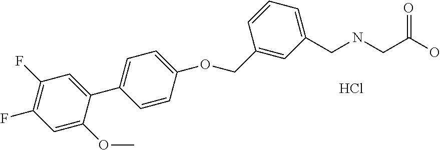

A mixture of 3-(4′,5′-Difluoro-2′-methoxy-biphenyl-4-yloxymethyl)-benzylamino]-acetic acid ethyl ester hydrochloride (130 mg, 0.24 mmol), and lithium hydroxide solution (0.5N, 3 mL) in THF (4 mL) was stirred for 3 h. The reaction mixture was diluted with ethyl acetate and washed with dilute hydrochloric acid. The organic layer was washed with water and brine and dried over sodium sulfate. Solvent was removed and the oil product was converted to the hydrochloride salt with hydrogen chloride in ether (3M, 4 mL) to afford [3-(4′,5′-difluoro-2′-methoxy-biphenyl-4-yloxymethyl)-benzylamino]-acetic acid (75.0 mg, 63% yield) as a white solid. LRMS calcd for C₂₃H₂₁F₂NO₄ (m/e) 414.14 (M+H), obsd 414.1 (ES+).

### Example 38

### 1,1-Dioxo-2-[3-(2′,4′,5′-trifluoro-biphenyl-4-yloxymethyl)-benzyl]-1lambda*6*-isothiazolidine-3-carboxylic acid

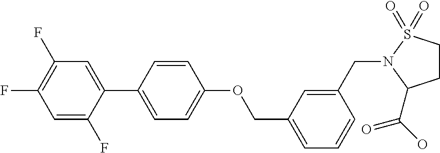

A mixture of 4′-(3-bromomethyl-benzyloxy)-2,4,5-trifluoro-biphenyl (194 mg, 0.48 mmol), 1,1-dioxo-isothiazolidine-3-carboxylic acid methyl ester (102 mg, 0.57 mmol), and potassium carbonate (103 mg, 0.75 mmol), in DMF (10 mL) was stirred at room temperature for 3 h. Solvent was evaporated and the residue was extracted with ethyl acetate and water. The organic layer was washed with brine and dried over sodium sulfate. After the evaporation of solvent, the residue was purified on a flash chromatography column with ethyl acetate in hexanes to yield 1,1-dioxo-2-[3-(2′,4′,5′-trifluoro-biphenyl-4-yloxymethyl)-benzyl]-1lambda*6*-isothiazolidine-3-carboxylic acid methyl ester (175 mg, 73% yield).

1,1-Dioxo-2-[3-(2′,4′,5′-trifluoro-biphenyl-4-yloxymethyl)-benzyl]-1lambda*6*-isothiazolidine-3-carboxylic acid methyl ester (175 mg, 0.35 mmol) was dissolved in THF (5 mL) and treated with lithium hydroxide solution (0.5N, 2 mL). The mixture was stirred at room temperature for 3 hrs and solvent was evaporated. The residue was extracted with ethyl acetate and 1N hydrochloric acid. The organic layer was washed with water and brine, dried over sodium sulfate and solvent was removed. The residue was treated with a 1:1 ratio of acetonitrile and water (10 mL) and the mixture was lyophilized to afford 1,1-dioxo-2-[3-(2′,4′,5′-trifluoro-biphenyl-4-yloxymethyl)-benzyl]-1lambda*6*-isothiazolidine-3-carboxylic acid (167 mg, 98% yield). LRMS calcd for C₂₄H₂₀F₃NO₅S (m/e) 490.10 (M−H), obsd 490.0 (ES−).

### Example 39

### 2-[3-(4′,5′-Difluoro-2′-methyl-biphenyl-4-yloxymethyl)-benzyl]-1,1-dioxo-1lambda*6*-isothiazolidine-3-carboxylic acid

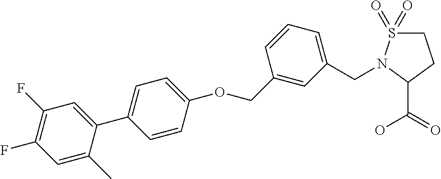

With a method similar to that used for the preparation of 1,1-dioxo-2-[3-(2′,4′,5′-trifluoro-biphenyl-4-yloxymethyl)-benzyl]-1lambda*6*-isothiazolidine-3-carboxylic acid, 2-[3-(4′,5′-difluoro-2′-methyl-biphenyl-4-yloxymethyl)-benzyl]-1,1-dioxo-1lambda*6*-isothiazolidine-3-carboxylic acid was prepared from 4′-(3-bromomethyl-benzyloxy)-4,5-difluoro-2-methyl-biphenyl and 1,1-dioxo-isothiazolidine-3-carboxylic acid methyl ester. LRMS calcd for C₂₅H₂₃F₂NO₅S (m/e) 486.13 (M−H), obsd 486.0 (ES−).

### Example 40

### 2-[3-(2′-Chloro-4′,5′-difluoro-biphenyl-4-yloxymethyl)-benzyl]-1,1-dioxo-1lambda*6*-isothiazolidine-3-carboxylic acid

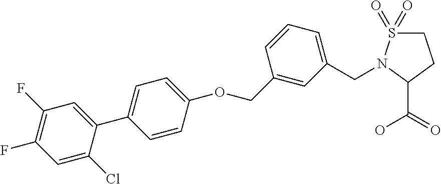

With a method similar to that used for the preparation of 1,1-dioxo-2-[3-(2′,4′,5′-trifluoro-biphenyl-4-yloxymethyl)-benzyl]-1lambda*6*-isothiazolidine-3-carboxylic acid, 2-[3-(2′-chloro-4′,5′-difluoro-biphenyl-4-yloxymethyl)-benzyl]-1,1-dioxo-1lambda*6*-isothiazolidine-3-carboxylic acid was prepared from 4′-(3-bromomethyl-benzyloxy)-2-chloro-4,5-difluoro-biphenyl and 1,1-dioxo-isothiazolidine-3-carboxylic acid methyl ester. LC-MS calcd for C₂₄H₂₀ClF₂NO₅S (m/e) 506.07 (M−H), obsd 505.9 (ES−).

### Example 41

### 2-[3-(2′,4′-Difluoro-biphenyl-4-yloxymethyl)-4-fluoro-benzyl]-1,1-dioxo-1lambda*6*-isothiazolidine-3-carboxylic acid

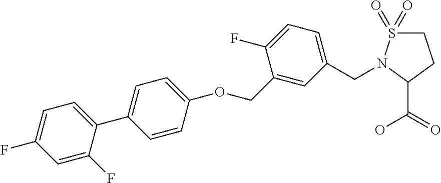

With a method similar to that used for the preparation of 1,1-dioxo-2-[3-(2′,4′,5′-trifluoro-biphenyl-4-yloxymethyl)-benzyl]-1lambda*6*-isothiazolidine-3-carboxylic acid, 2-[3-(2′,4′-difluoro-biphenyl-4-yloxymethyl)-4-fluoro-benzyl]-1,1-dioxo-1lambda*6*-isothiazolidine-3-carboxylic acid was prepared from 4′-(5-bromomethyl-2-fluoro-benzyloxy)-2,4-difluoro-biphenyl and 1,1-dioxo-isothiazolidine-3-carboxylic acid methyl ester. LRMS calcd for C₂₄H₂₀F₃NO₅S (m/e) 490.10 (M−H), obsd 490.0 (ES−).

### Example 42

### 2-[5-(2′,4′-Difluoro-biphenyl-4-yloxymethyl)-2-fluoro-benzyl]-1,1-dioxo-1lambda*6*-isothiazolidine-3-carboxylic acid

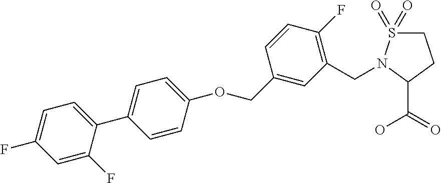

With a method similar to that used for the preparation of 1,1-dioxo-2-[3-(2′,4′,5′-trifluoro-biphenyl-4-yloxymethyl)-benzyl]-1lambda*6*-isothiazolidine-3-carboxylic acid, 2-[5-(2′,4′-difluoro-biphenyl-4-yloxymethyl)-2-fluoro-benzyl]-1,1-dioxo-1lambda*6*-isothiazolidine-3-carboxylic acid was prepared from 4′-(3-bromomethyl-4-fluoro-benzyloxy)-2,4-difluoro-biphenyl and 1,1-dioxo-isothiazolidine-3-carboxylic acid methyl ester. LRMS calcd for C₂₄H₂₀F₃NO₅S (m/e) 490.10 (M−H), obsd 490.0 (ES−).

### Example 43

### 2-[3-(4′,5′-Difluoro-2′-methoxy-biphenyl-4-yloxymethyl)-benzyl]-1,1-dioxo-1lambda*6*-isothiazolidine-3-carboxylic acid

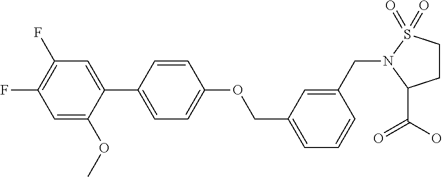

With a method similar to that used for the preparation of 1,1-dioxo-2-[3-(2′,4′,5′-trifluoro-biphenyl-4-yloxymethyl)-benzyl]-1lambda*6*-isothiazolidine-3-carboxylic acid, 2-[3-(4′,5′-difluoro-2′-methoxy-biphenyl-4-yloxymethyl)-benzyl]-1,1-dioxo-1lambda*6*-isothiazolidine-3-carboxylic acid was prepared from 4′-(3-bromomethyl-benzyloxy)-4,5-difluoro-2-methoxy-biphenyl and 1,1-dioxo-isothiazolidine-3-carboxylic acid methyl ester. LC-MS calcd for C₂₅H₂₃F₂NO₆S (m/e) 502.12 (M−H), obsd 502.0 (ES−).

### Example 44

### (R)-2-[3-(4′,5′-Difluoro-2′-methoxy-biphenyl-4-yloxymethyl)-benzyl]-1,1-dioxo-1lambda*6*-isothiazolidine-3-carboxylic acid

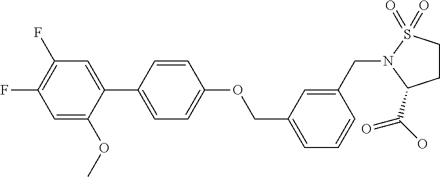

Racemic 2-[3-(4′,5′-difluoro-2′-methoxy-biphenyl-4-yloxymethyl)-benzyl]-1,1-dioxo-1lambda*6*-isothiazolidine-3-carboxylic acid was separated by preparative SFC (super critical fluid chromatography, Burger Multigram-II) in multiple runs on a Diacel AD column (3×25 cm, 35% methanol, 30° C., rate 70 mL/min, pressure 100 bar CO₂, 25 mg compound loading for each run). The first band to elute was evaporated to give (R)-2-[3-(4′,5′-difluoro-2′-methoxy-biphenyl-4-yloxymethyl)-benzyl]-1,1-dioxo-1lambda*6*-isothiazolidine-3-carboxylic acid as a white foam. LC-MS calcd for C₂₅H₂₃F₂NO₆S (m/e) 502.12 (M−H), obsd 502.0 (ES−).

### Example 45

### (S)-2-[3-(4′,5′-Difluoro-2′-methoxy-biphenyl-4-yloxymethyl)-benzyl]-1,1-dioxo-1lambda*6*-isothiazolidine-3-carboxylic acid

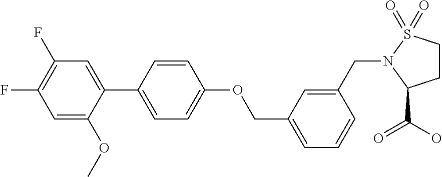

Racemic 2-[3-(4′,5′-difluoro-2′-methoxy-biphenyl-4-yloxymethyl)-benzyl]-1,1-dioxo-1lambda*6*-isothiazolidine-3-carboxylic acid was separated by preparative SFC (super critical fluid chromatography) in multiple runs on a Diacel AD column (3×25 cm, 35% methanol, 30° C., rate 70 mL/min, pressure 100 bar CO₂, 25 mg compound loading for each run). The second band to elute was evaporated to give (S)-2-[3-(4′,5′-difluoro-2′-methoxy-biphenyl-4-yloxymethyl)-benzyl]-1,1-dioxo-1lambda*6*-isothiazolidine-3-carboxylic acid as a white foam. LC-MS calcd for C₂₅H₂₃F₂NO₆S (m/e) 502.12 (M−H), obsd 502.0 (ES−).

### Example 46

### (S)-1,1-Dioxo-2-[3-(2′,4′,5′-trifluoro-biphenyl-4-yloxymethyl)-benzyl]-1lambda*6*-isothiazolidine-3-carboxylic acid

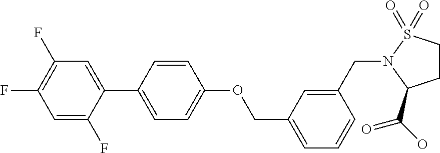

To a DMF (5 mL) solution containing 4′-(3-bromomethyl-benzyloxy)-2,4,5-trifluoro-biphenyl (213.6 mg, 0.52 mmol) and (S)-1,1-dioxo-isothiazolidine-3-carboxylic acid tert-butyl ester (116 mg, 0.52 mmol) was added potassium carbonate (145 mg, 1.05 mmol). The mixture was stirred at room temperature for 3 hrs and extracted with ethyl acetate and water. The organic layer was washed with water and brine, dried over sodium sulfate and solvents were evaporated. The residue was purified through flash column chromatography (23 g silica gel, 5% to 60% ethyl acetate in hexanes) to give oily material as (S)-1,1-dioxo-2-[3-(2′,4′,5′-trifluoro-biphenyl-4-yloxymethyl)-benzyl]-1lambda*6*-isothiazolidine-3-carboxylic acid tert-butyl ester (262 mg, 91.3% yield).

(S)-1,1-dioxo-2-[3-(2′,4′,5′-trifluoro-biphenyl-4-yloxymethyl)-benzyl]-1lambda*6*-isothiazolidine-3-carboxylic acid tert-butyl ester (262 mg, 0.48 mmol) was dissolved in dichloromethane (3 mL) and trifluoroacetic acid (6 mL) was added. The mixture was stirred at room temperature for 2 hrs and solvents were evaporated. The residue was treated with toluene (10 mL) and evaporated to dryness. The resulting material was extracted with ethyl acetate and water. The organic layer was dried over sodium sulfate and solvent was evaporated. The residue was triturated with ether and the solid was filtered to give (S)-1,1-dioxo-2-[3-(2′,4′,5′-trifluoro-biphenyl-4-yloxymethyl)-benzyl]-1lambda*6*-isothiazolidine-3-carboxylic acid (178 mg, 75% yield). LC-MS calcd for C₂₄H₂₀F₃NO₅S (m/e) 490.10 (M−H), obsd 490.0 (ES−).

This compound was analyzed on a chiral SFC (super critical fluid chromatography, chiral AD column) and compared with the corresponding racemate (prepared in Example 38). It showed 100% enantiomeric purity and the retention time was identical to the second fraction of the corresponding racemate.

### Example 47

### (R)-1,1-Dioxo-2-[3-(2′,4′,5′-trifluoro-biphenyl-4-yloxymethyl)-benzyl]-1lambda*6*-isothiazolidine-3-carboxylic acid

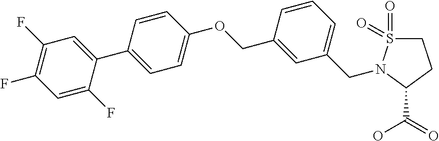

Racemic 1,1-dioxo-2-[3-(2′,4′,5′-trifluoro-biphenyl-4-yloxymethyl)-benzyl]-1lambda*6*-isothiazolidine-3-carboxylic acid (Example 38) was separated by preparative SFC (super critical fluid chromatography, Burger Multigram-II) in multiple runs on a Diacel AD column (3×25 cm, 35% methanol, 30° C., rate 70 mL/min, pressure 100 bar CO₂, detector 220 nm, 25 mg compound loading for each run). The first band to elute was evaporated to give (R)-1,1-dioxo-2-[3-(2′,4′,5′-trifluoro-biphenyl-4-yloxymethyl)-benzyl]-1lambda*6*-isothiazolidine-3-carboxylic acid as a white foam. LC-MS calcd for C₂₄H₂₀F₃NO₅S (m/e) 490.10 (M−H), obsd 490.0 (ES−).

### Example 48

### (S)-2-[3-(4′,5′-Difluoro-2′-methyl-biphenyl-4-yloxymethyl)-benzyl]-1,1-dioxo-1lambda*6*-isothiazolidine-3-carboxylic acid

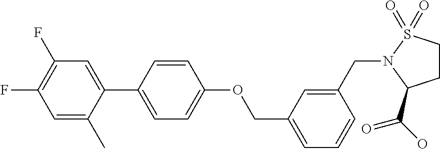

Racemic 2-[3-(4′,5′-difluoro-2′-methyl-biphenyl-4-yloxymethyl)-benzyl]-1,1-dioxo-1lambda*6*-isothiazolidine-3-carboxylic acid (Example 39) was separated by preparative SFC (super critical fluid chromatography, Burger Multigram-II) in multiple runs on a Diacel AD column (3×25 cm, 35% methanol, 30° C., rate 70 mL/min, pressure 100 bar CO₂, detector 220 nm, 25 mg compound loading for each run). The second fraction to elute was evaporated to give (S)-2-[3-(4′,5′-difluoro-2′-methyl-biphenyl-4-yloxymethyl)-benzyl]-1,1-dioxo-1lambda*6*-isothiazolidine-3-carboxylic acid as a white foam. LC-MS calcd for C₂₅H₂₃F₂NO₅S (m/e) 486.13 (M−H), obsd 486.0 (ES−).

### Example 49

### (R)-2-[3-(4′,5′-Difluoro-2′-methyl-biphenyl-4-yloxymethyl)-benzyl]-1,1-dioxo-1lambda*6*-isothiazolidine-3-carboxylic acid

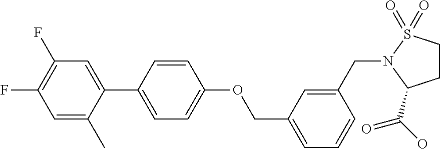

With the same method as described for the preparation of (S)-1,1-dioxo-2-[3-(2′,4′,5′-trifluoro-biphenyl-4-yloxymethyl)-benzyl]-1lambda*6*-isothiazolidine-3-carboxylic acid (Example 46), (R)-2-[3-(4′,5′-difluoro-2′-methyl-biphenyl-4-yloxymethyl)-benzyl]-1,1-dioxo-1lambda*6*-isothiazolidine-3-carboxylic acid was prepared from 4′-(3-bromomethyl-benzyloxy)-4,5-difluoro-2-methyl-biphenyl and (R)-1,1-dioxo-isothiazolidine-3-carboxylic acid tert-butyl ester. LC-MS calcd for C₂₅H₂₃F₂NO₅S (m/e) 486.13 (M−H), obsd 486.0 (ES−).

### Example 50

### (R)-2-[3-(2′-Chloro-4′,5′-difluoro-biphenyl-4-yloxymethyl)-benzyl]-1,1-dioxo-1lambda*6*-isothiazolidine-3-carboxylic acid

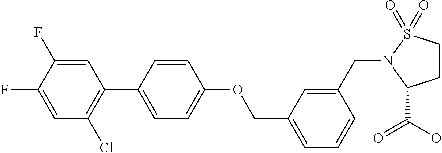

Racemic 2-[3-(2′-chloro-4′,5′-difluoro-biphenyl-4-yloxymethyl)-benzyl]-1,1-dioxo-1lambda*6*-isothiazolidine-3-carboxylic acid (Example 40) was separated by preparative SFC (super critical fluid chromatography, Burger Multigram-II) in multiple runs on a Diacel AD column (3×25 cm, 35% methanol, 30° C., rate 70 mL/min, pressure 100 bar CO₂, detector 220 nm, 25 mg compound loading for each run). The first fraction to elute was evaporated to give (R)-2-[3-(2′-chloro-4′,5′-difluoro-biphenyl-4-yloxymethyl)-benzyl]-1,1-dioxo-1lambda*6*-isothiazolidine-3-carboxylic acid as a white foam. LC-MS calcd for C₂₄H₂₀ClF₂NO₅S (m/e) 506.07 (M−H), obsd 506.0 (ES−).

### Example 51

### (S)-2-[3-(2′-Chloro-4′,5′-difluoro-biphenyl-4-yloxymethyl)-benzyl]-1,1-dioxo-1lambda*6*-isothiazolidine-3-carboxylic acid

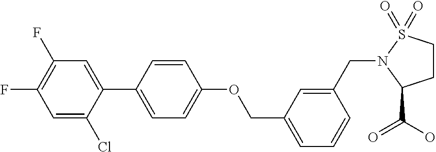

Racemic 2-[3-(2′-chloro-4′,5′-difluoro-biphenyl-4-yloxymethyl)-benzyl]-1,1-dioxo-1lambda*6*-isothiazolidine-3-carboxylic acid (Example 40) was separated by preparative SFC (super critical fluid chromatography, Burger Multigram-II) in multiple runs on a Diacel AD column (3×25 cm, 35% methanol, 30° C., rate 70 mL/min, pressure 100 bar CO₂, detector 220 nm, 25 mg compound loading for each run). The second fraction to elute was evaporated to give (S)-2-[3-(2′-chloro-4′,5′-difluoro-biphenyl-4-yloxymethyl)-benzyl]-1,1-dioxo-1lambda*6*-isothiazolidine-3-carboxylic acid as a white foam. LC-MS calcd for C₂₄H₂₀ClF₂NO₅S (m/e) 506.07 (M−H), obsd 506.0 (ES−).

### Example 52

### (S)-1-[3-(4′,5′-Difluoro-2′-methoxy-biphenyl-4-yloxymethyl)-benzyl]-5-oxo-pyrrolidine-2-carboxylic acid

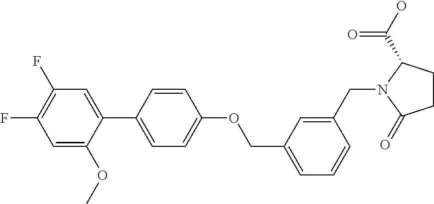

L-glutamic acid diethyl ester hydrochloride (338 mg, 1.4 mmol) was mixed with 3-(4′,5′-difluoro-2′-methoxy-biphenyl-4-yloxymethyl)-benzaldehyde (250 mg, 0.70 mmol) in methanol (12 mL) and THF (3 mL). Acetic acid (0.1 mL) was added and the solution was stirred for 10 minutes. Sodium triacetoxyboronhydride (740 mg, 5.0 eq) was added in five portions over 2 hrs. The mixture was stirred at room temperature for 48 hrs and solvents were evaporated. The residue was extracted with ethyl acetate and 1N hydrochloric acid solution. The organic layer was washed with water and concentrated sodium bicarbonate solution. After the evaporation of solvent, the residue was purified on a flash chromatography column eluted with ethyl acetate in hexanes to give a colorless oil as (S)-1-[3-(4′,5′-difluoro-2′-methoxy-biphenyl-4-yloxymethyl)-benzyl]-5-oxo-pyrrolidine-2-carboxylic acid ethyl ester (240 mg, 68.7% yield).

(S)-1-[3-(4′,5′-Difluoro-2′-methoxy-biphenyl-4-yloxymethyl)-benzyl]-5-oxo-pyrrolidine-2-carboxylic acid ethyl ester (173 mg, 0.35 mmol) was dissolved in THF (4 mL) and lithium hydroxide solution (0.5N, 1.0 mL) was added followed by addition of methanol (0.5 mL). The clear solution was stirred at room temperature for 1 hr and solvents were evaporated. The residue was dissolved in water (10 mL) and treated with 1N hydrochloric acid (1 mL). The white solid was filtered and dried to give (S)-1-[3-(4′,5′-difluoro-2′-methoxy-biphenyl-4-yloxymethyl)-benzyl]-5-oxo-pyrrolidine-2-carboxylic acid (156 mg, 96.7% yield). LC-MS calcd for C₂₆H₂₃F₂NO₅ (m/e) 468.15 (M+H), obsd 468.0 (ES+).

### Example 53

### (S)-5-Oxo-1-[3-(2′,4′,5′-trifluoro-biphenyl-4-yloxymethyl)-benzyl]-pyrrolidine-2-carboxylic acid

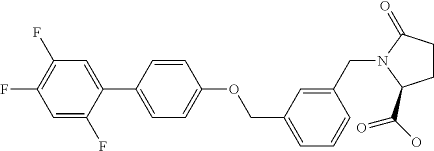

With the same method as described for the preparation of (S)-1-[3-(4′,5′-difluoro-2′-methoxy-biphenyl-4-yloxymethyl)-benzyl]-5-oxo-pyrrolidine-2-carboxylic acid, (S)-5-oxo-1-[3-(2′,4′,5′-trifluoro-biphenyl-4-yloxymethyl)-benzyl]-pyrrolidine-2-carboxylic acid was prepared from L-glutamic acid diethyl ester hydrochloride and 3-(2′,4′,5′-trifluoro-biphenyl-4-yloxymethyl)-benzaldehyde. LC-MC clacd for C₂₅H₂₀F₃NO₄ (m/e) 454.13 (M−H), obsd 454.0 (ES−).

### Example 54

### (S)-1-[3-(2′,4′-Difluoro-biphenyl-4-yloxymethyl)-benzyl]-pyrrolidine-2-carboxylic acid; trifluoro-acetic acid salt

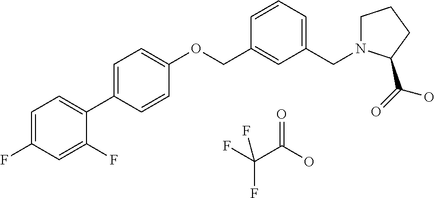

2′,4′-Difluoro-biphenyl-4-ol (114.7 mg, 0.556 mmol), 1,3-bis-bromomethyl-benzene (146.8 mg, 0.556 mmol) and dried, finely ground potassium carbonate (77 mg, 0.556 mmol) in 5 mL acetone were heated at 65° C. for 6.5 hrs. The mixture was cooled, diluted with 20 mL ethyl acetate, washed with 5 mL H₂O, 5 mL saturated NaCl and dried over MgSO₄. The organic solvents were evaporated to yield a white solid that was purified by flash chromatography to yield 50 mg of 4′-(3-bromomethyl-benzyloxy)-2,4-difluoro-biphenyl.

4′-(3-Bromomethyl-benzyloxy)-2,4-difluoro-biphenyl (7.5 mg, 0.19 mmol), L-proline (4.4 mg, 0.038 mmol) and DIPEA (13.7 uL, 0.77 mmol) in 1 mL acetonitrile were heated at 65° C. for 5 hrs. The mixture was cooled, evaporated to dryness and redissolved in 1 mL AcOH. The crude solution was applied to a C₁₈ Sep-Pak® cartridge and eluted with a step gradient of 1% TFA/CH₃CN in H₂O in 10% increments. The product eluted in the 50% CH₃CN/H₂O/1% TFA fraction. The band was evaporated to dryness to yield 2 mg of (S)-1-[3-(2′,4′-difluoro-biphenyl-4-yloxymethyl)-benzyl]-pyrrolidine-2-carboxylic acid trifluoro-acetic acid salt as a clear gum. LC-MS (ES) calculated for C₂₅H₂₃F₂NO₃, 423.46; found m/z 424 [M+H]⁺.

### Example 55

### (R)-2-[3-(4′,5′-Difluoro-2′-methoxy-biphenyl-4-yloxymethyl)-benzyl]-1,1-dioxo-1lambda*6*-[1,2]thiazinane-3-carboxylic acid

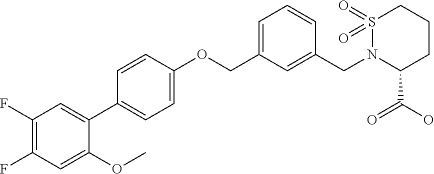

The 4′-(3-Bromomethyl-benzyloxy)-4,5-difluoro-2-methoxy-biphenyl, (120 mg, 0.29 mmol), 1,1-dioxo-1lambda*6*-[1,2]thiazinane-3-carboxylic acid tert-butyl ester (67.3 mg, 0.29 mmol) and potassium carbonate (79.1 mg, 0.57 mmol) were suspended in 5 ml of DMF. The mixture was stirred at room temperature overnight and then diluted with ethyl acetate and water. The organic layer was dried and solvents were evaporated. The crude product was purified by using an ISCO (40 g silica) column chromatography eluting with 5-30% ethyl acetate in hexanes to obtain 2-{[3-(4′,5′-difluoro-2′-methoxy-biphenyl-4-yloxymethyl)-benzyl]-1,1-dioxo-1lambda*6*-[1,2]thiazinane-3-carboxylic acid tert-butyl ester as a fluffy solid (120 mg, 73%). LC-MS (ES) calculated for C₃₀H₃₃F₂NO₆S, 573.66; found m/z 596.1 [M+Na]⁺.

Methylene chloride (2 mL) was added to the 2-{[3-(4′,5′-difluoro-2′-methoxy-biphenyl-4-yloxymethyl)-benzyl]-1,1-dioxo-1lambda*6*-[1,2]thiazinane-3-carboxylic acid tert-butyl ester to afford a clear solution. TFA (1 mL) was added and the mixture was stirred at room temperature for 1 h. Toluene (2×10 mL) was added and the mixture was concentrated to dryness.

Racemic 2-[3-(4′,5′-difluoro-2′-methoxy-biphenyl-4-yloxymethyl)-benzyl]-1,1-dioxo-1lambda*6*-[1,2]thiazinane-3-carboxylic acid was separated by preparative SFC in multiple runs in on a Diacel OJ column (50% MeOH, 30° C., 70 mL/min and 100 bar CO₂). The first band to elute was evaporated to give 41.6 mg (40%) of (R)-2-[3-(4′,5′-difluoro-2′-methoxy-biphenyl-4-yloxymethyl)-benzyl]-1,1-dioxo-1lambda*6*-[1,2]thiazinane-3-carboxylic acid as a white foam. LC-MS (ES) calculated for C₂₆H₂₅F₂NO₆S, 517.55; found m/z 516.2 [M−H]⁻.

### Example 56

### (S)-2-[3-(4′,5′-Difluoro-2′-methoxy-biphenyl-4-yloxymethyl)-benzyl]-1,1-dioxo-1lambda*6*-[1,2]thiazinane-3-carboxylic acid

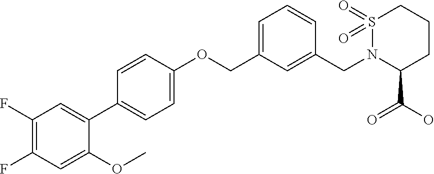

The second band to elute from the above SFC purification of racemic 2-[3-(4′,5′-difluoro-2′-methoxy-biphenyl-4-yloxymethyl)-benzyl]-1,1-dioxo-1lambda*6*-[1,2]thiazinane-3-carboxylic acid was evaporated to give 40.0 mg (39%) of (S)-2-[3-(4′,5′-difluoro-2′-methoxy-biphenyl-4-yloxymethyl)-benzyl]-1,1-dioxo-1lambda*6*-[1,2]thiazinane-3-carboxylic acid as a white foam. LC-MS (ES) calculated for C₂₆H₂₅F₂NO₆S, 517.55; found m/z 518.1 [M+H]⁺.

### Example 57

### (R)-1,1-Dioxo-2-[3-(2′,4′,5′-trifluoro-biphenyl-4-yloxymethyl)-benzyl]-1lambda*6*-[1,2]thiazinane-3-carboxylic acid

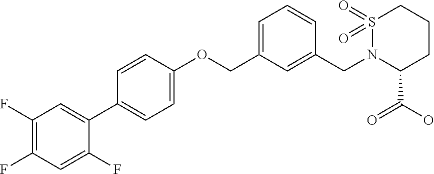

4′-(3-Bromomethyl-benzyloxy)-2,4,5-trifluoro-biphenyl, (110 mg, 0.27 mmol), 1,1-dioxo-1lambda*6*-[1,2]thiazinane-3-carboxylic acid tert-butyl ester (63.5 mg, 0.27 mmol) and potassium carbonate (74.6 mg, 0.54 mmol) were suspended in 5 ml of DMF. The mixture was stirred at room temperature overnight and then diluted with ethyl acetate and water. The organic layer was dried and solvents were evaporated. The crude product was purified by using an ISCO (40 g silica) column chromatography, eluting with 5-30% ethyl acetate in hexanes to obtain 1,1-dioxo-2-[3-(2′,4′,5′-trifluoro-biphenyl-4-yloxymethyl)-benzyl]-1lambda*6*-[1,2]thiazinane-3-carboxylic acid tert-butyl ester as a fluffy solid (58.9 mg, 38.8%). ES-MS calcd for C₂₉H₃₀F₃NO₅S (m/e) 561.6, obsd 560.1 (M−H).

Methylene chloride (2 mL) was added to the 1,1-dioxo-2-[3-(2′,4′,5′-trifluoro-biphenyl-4-yloxymethyl)-benzyl]-1lambda*6*-[1,2]thiazinane-3-carboxylic acid tert-butyl ester to afford a clear solution. TFA (1 mL) was added and the mixture was stirred at room temperature for 1 h. Toluene (2×10 mL) was added and the mixture was concentrated to dryness.

Racemic 1,1-dioxo-2-[3-(2′,4′,5′-trifluoro-biphenyl-4-yloxymethyl)-benzyl]-1lambda*6*-[1,2]thiazinane-3-carboxylic acid was separated by preparative SFC in multiple runs in on a Diacel OJ column (50% Hexane/EtOH, 30° C., 70 mL/min and 100 bar CO₂). The first band to elute was evaporated to give 10 mg (7.3%) of (R)-1,1-dioxo-2-[3-(2′,4′,5′-trifluoro-biphenyl-4-yloxymethyl)-benzyl]-1lambda*6*-[1,2]thiazinane-3-carboxylic acid as a white foam. LC-MS (ES) calculated for C₂₅H₂₂F₃NO₅S, 505.2; found m/z 504.1 [M−H]⁻.

### Example 58

### (S)-1,1-Dioxo-2-[3-(2′,4′,5′-trifluoro-biphenyl-4-yloxymethyl)-benzyl]-1lambda*6*-[1,2]thiazinane-3-carboxylic acid

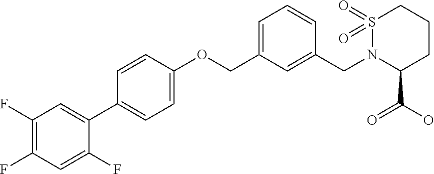

The second band to elute from the above SFC purification of racemic 2-[3-(2′,4′,5′-trifluoro-biphenyl-4-yloxymethyl)-benzyl]-1,1-dioxo-1lambda*6*-[1,2]thiazinane-3-carboxylic acid was evaporated to give 8.8 mg (6.5%) of (S)-1,1-dioxo-2-[3-(2′,4′,5′-trifluoro-biphenyl-4-yloxymethyl)-benzyl]-1lambda*6*-[1,2]thiazinane-3-carboxylic acid as a white foam. LC-MS (ES) calculated for C₂₅H₂₂F₃NO₅S, 505.2; found m/z 504.2 [M−H]⁻.

### Example 59

### (R)-1,1-Dioxo-2-[2-(2′,4′,5′-trifluoro-biphenyl-4-yloxymethyl)-thiazol-4-ylmethyl]-1lambda*V-isothiazolidine-3-carboxylic acid

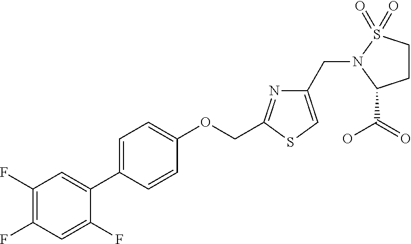

A mixture of 1,1-dioxo-1lambda*6*-isothiazolidine-3-carboxylic acid ethyl ester (174 mg, 0.901 mmol) and potassium carbonate (336 mg, 2.43 mmol) in DMF (4 mL) was heated to 50° C. and stirred for 2 min. A solution of 4-iodomethyl-2-(2′,4′,5′-trifluoro-biphenyl-4-yloxymethyl)-thiazole (373 mg, 0.809 mmol) in DMF (6 mL) was added and the reaction mixture was heated at 50° C. for 45 min. The reaction mixture was cooled, diluted with water and aqueous 2N HCl and extracted with ethyl acetate (3×). The organic layers were combined, washed with brine, dried (MgSO₄), filtered, concentrated, flash chromatographed (silica, 120 g, 40% ethyl acetate in hexanes) to give 1,1-dioxo-2-[2-(2′,4′,5′-trifluoro-biphenyl-4-yloxymethyl)-thiazol-4-ylmethyl]-1lambda*6*-isothiazolidine-3-carboxylic acid ethyl ester (312.1 mg, 73.3%) as a colorless oil. LC-MS (ES) calculated for C₂₃H₂₁F₃N₂O₅S₂, 526.56; found m/z 527 [M+H]⁺.

A solution of 1,1-dioxo-2-[2-(2′,4′,5′-trifluoro-biphenyl-4-yloxymethyl)-thiazol-4-ylmethyl]-1lambda*6*-isothiazolidine-3-carboxylic acid ethyl ester (308 mg, 0.585 mmol), lithium hydroxide monohydrate (121 mg, 2.88 mmol), THF (4 mL) and water (4 mL) was stirred at room temperature for 16 h. The reaction mixture was diluted with water, acidified with aqueous 2N HCl and extracted with ethyl acetate (3×25 mL). The organic layers were combined, washed with brine (25 mL), dried (MgSO₄), filtered and concentrated to give 1,1-dioxo-2-[2-(2′,4′,5′-trifluoro-biphenyl-4-yloxymethyl)-thiazol-4-ylmethyl]-1lambda*6*-isothiazolidine-3-carboxylic acid (259.9 mg, 89.1%) as a white solid. LC-MS (ES) calculated for C₂₁H₁₇F₃N₂O₅S₂, 498.50; found m/z 499 [M+H]

Racemic 1,1-dioxo-2-[2-(2′,4′,5′-trifluoro-biphenyl-4-yloxymethyl)-thiazol-4-ylmethyl]-1lambda*6*-isothiazolidine-3-carboxylic acid (200 mg) was separated by preparative SFC in multiple runs in on a RR Whelko column (Regis Technologies) (45% MeOH, 30° C., 2 mL/min and 100 bar CO₂) The first band to elute was evaporated to give 74.1 mg (37%) of (R)-1,1-dioxo-2-[2-(2′,4′,5′-trifluoro-biphenyl-4-yloxymethyl)-thiazol-4-ylmethyl]-1lambda*6*-isothiazolidine-3-carboxylic acid as a white foam. LC-MS (ES) calculated for C₂₁H₁₇F₃N₂O₅S₂, 498.50; found m/z 499 [M+H]⁺.

### Example 60

### (S)-1,1-Dioxo-2-[2-(2′,4′,5′-trifluoro-biphenyl-4-yloxymethyl)-thiazol-4-ylmethyl]-1lambda*6*-isothiazolidine-3-carboxylic acid

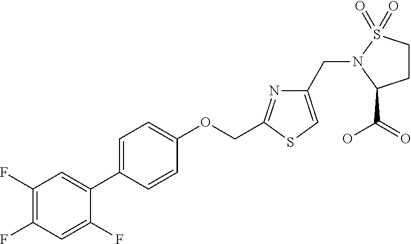

The second band to elute from the above SFC purification of racemic 1,1-dioxo-2-[2-(2′,4′,5′-trifluoro-biphenyl-4-yloxymethyl)-thiazol-4-ylmethyl]-1lambda*6*-isothiazolidine-3-carboxylic acid (200 mg) was evaporated to give 93 mg (47%) of (S)-1,1-dioxo-2-[2-(2′,4′,5′-trifluoro-biphenyl-4-yloxymethyl)-thiazol-4-ylmethyl]-1lambda*6*-isothiazolidine-3-carboxylic acid as a white solid. LC-MS (ES) calculated for C₂₁H₁₇F₃N₂O₅S₂, 498.50; found m/z 499 [M+H]⁺.

### Example 61

### (R)-2-[2-(4′,5′-Difluoro-2′-methoxy-biphenyl-4-yloxymethyl)-thiazol-4-ylmethyl]-1,1-dioxo-1lambda*6*-isothiazolidine-3-carboxylic acid

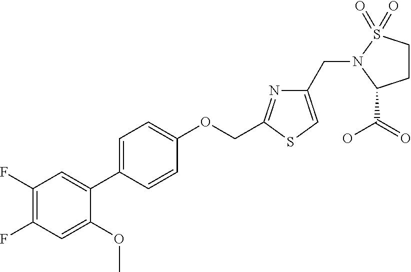

A mixture of 1,1-dioxo-1lambda*6*-isothiazolidine-3-carboxylic acid ethyl ester (100 mg, 0.518 mmol) and potassium carbonate (193 mg, 1.396 mmol) in DMF (4 mL) was heated to 40° C. and stirred for 2 min. A solution of 2-(4′,5′-difluoro-2′-methoxy-biphenyl-4-yloxymethyl)-4-iodomethyl-thiazole (220 mg, 0.465 mmol) in DMF (3 mL) was added and the reaction mixture was heated at 50° C. for 1 hr. The reaction mixture was cooled, diluted with water and aqueous 2N HCl and extracted with ethyl acetate (3×). The organic layers were combined, washed with brine, dried (MgSO₄), filtered, concentrated, to give 1,1-dioxo-2-[2-(2′,4′,5′-trifluoro-biphenyl-4-yloxymethyl)-thiazol-4-ylmethyl]-1lambda*6*-isothiazolidine-3-carboxylic acid ethyl ester (269 mg) which was used without further purification. LC-MS (ES) calculated for C₂₄H₂₄F₂N₂O₆S₂, 538.28; found m/z 539 [M+H]⁺

A solution of 1,1-dioxo-2-[2-(4′,5′-difluoro-2′-methoxy biphenyl-4-yloxymethyl)-thiazol-4-ylmethyl]-1lambda*6*-isothiazolidine-3-carboxylic acid ethyl ester (269 mg, 0.501 mmol), lithium hydroxide monohydrate (120 mg, 2.88 mmol), THF (4 mL) and water (4 mL) was stirred at room temperature for 16 h. The reaction mixture was diluted with water, acidified with aqueous 2N HCl and extracted with ethyl acetate (3×25 mL). The organic layers were combined, washed with brine (25 mL), dried (MgSO₄), filtered and concentrated to afford 1,1-dioxo-2-[2-(4′,5′-difluoro-2′-methoxy-biphenyl-4-yloxymethyl)-thiazol-4-ylmethyl]-1lambda*6*-isothiazolidine-3-carboxylic acid (194.9 mg, 76.1%) as a white solid.

Racemic 2-[2-(4′,5′-difluoro-2′-methoxy-biphenyl-4-yloxymethyl)-thiazol-4-ylmethyl]-1,1-dioxo-1lambda*6*-isothiazolidine-3-carboxylic acid (100 mg) was separated by preparative SFC in multiple runs in on a AD Diacel column (35% MeOH, 30° C., 70 mL/min and 100 bar CO₂). The first band to elute from the above SFC purification was evaporated to give (25.7, 26%) of (R)-2-[2-(4′,5′-difluoro-2′-methoxy-biphenyl-4-yloxymethyl)-thiazol-4-ylmethyl]-1,1-dioxo-1lambda*6*-isothiazolidine-3-carboxylic acid as a white solid. LC-MS (ES) calculated for C₂₂H₂₀F₂N₂O₆S₂, 510.54; found m/z 511 [M+H]⁺.

### Example 62

### (S)-2-[2-(4′,5′-Difluoro-2′-methoxy-biphenyl-4-yloxymethyl)-thiazol-4-ylmethyl]-1,1-dioxo-1lambda*6*-isothiazolidine-3-carboxylic acid

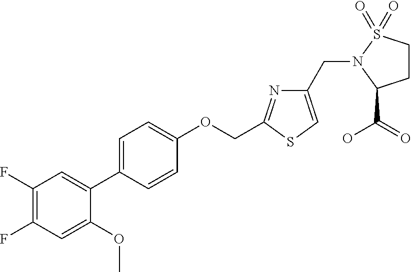

The second band to elute from the above SFC purification of racemic 2-[2-(4′,5′-difluoro-2′-methoxy-biphenyl-4-yloxymethyl)-thiazol-4-ylmethyl]-1,1-dioxo-1lambda*6*-isothiazolidine-3-carboxylic acid (100 mg) was evaporated to give (37.6, 38%) of (S)-2-[2-(4′,5′-difluoro-2′-methoxy-biphenyl-4-yloxymethyl)-thiazol-4-ylmethyl]-1,1-dioxo-1lambda*6*-isothiazolidine-3-carboxylic acid as a white solid. LC-MS (ES) calculated for C₂₂H₂₀F₂N₂O₆S₂, 510.54; found m/z 511 [M+H]⁺.

### Example 63

### 2-[5-(4′,5′-Difluoro-2′-methoxy-biphenyl-4-yloxymethyl)-pyridin-3-ylmethyl]-1,1-dioxo-1lambda*6*-isothiazolidine-3-carboxylic acid

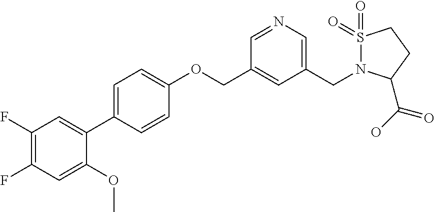

In a manner similar to above, from 3-(4′,5′-difluoro-2′-methoxy-biphenyl-4-yloxymethyl)-5-iodomethyl-pyridine there was produced 2-[5-(4′,5′-difluoro-2′-methoxy-biphenyl-4-yloxymethyl)-pyridin-3-ylmethyl]-1,1-dioxo-1lambda*6*-isothiazolidine-3-carboxylic acid ethyl ester. This ester was used without further purification.

From 2-[5-(4′,5′-difluoro-2′-methoxy-biphenyl-4-yloxymethyl)-pyridin-3-ylmethyl]-1,1-dioxo-1lambda*6*-isothiazolidine-3-carboxylic acid ethyl ester there was produced 2-[5-(4′,5′-difluoro-2′-methoxy-biphenyl-4-yloxymethyl)-pyridin-3-ylmethyl]-1,1-dioxo-1lambda*6*-isothiazolidine-3-carboxylic acid (1.03%) as a white solid. LC-MS (ES) calculated for C₂₄H₂₂F₂N₂O₆S, 504.51; found m/z 505 [M+H]⁺.

### Example 64

### 2-[5-(4′,5′-Difluoro-2′-methoxy-biphenyl-4-yloxymethyl)-pyridin-3-ylmethyl]-1,1-dioxo-1lambda*6*-isothiazolidine-3-carboxylic acid

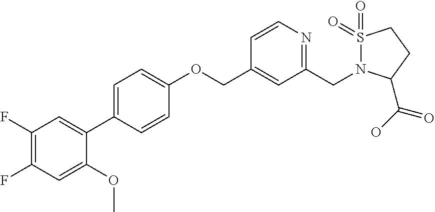

From 4-(4′,5′-difluoro-2′-methoxy-biphenyl-4-yloxymethyl)-2-iodomethyl-pyridine there was produced 2-[4-(4′,5′-difluoro-2′-methoxy-biphenyl-4-yloxymethyl)-pyridin-2-ylmethyl]-1,1-dioxo-lambda*6*-isothiazolidine-3-carboxylic acid ethyl ester which was used without further purification. LC-MS (ES) calculated for C₂₆H₂₆F₂N₂O₆S, 532.57; found m/z 533 [M+H]⁺.

From 2-[4-(4′,5′-difluoro-2′-methoxy-biphenyl-4-yloxymethyl)-pyridin-2-ylmethyl]-1,1-dioxo-lambda*6*-isothiazolidine-3-carboxylic acid ethyl ester there was produced 2-[4-(4′,5′-difluoro-2′-methoxy-biphenyl-4-yloxymethyl)-pyridin-2-ylmethyl]-1,1-dioxo-1lambda*6*-isothiazolidine-3-carboxylic (22.1 mg, 31%) as a white solid. LC-MS (ES) calculated for C₂₄H₂₂F₂N₂O₆S, 504.51; found m/z 505 [M+H]⁺.

### Example 65

### Glycogen Synthase (GS) Assay

The following tests were carried out in order to determine the activity of the compounds of formula (I).

Twelve μL per well of substrate solution containing glycogen (4.32 mg/ml), 2.67 mM UDP-glucose, 21.6 mM phospho(enol)pyruvate and 2.7 mM NADH in 30 mM glycylglycine, pH 7.3 buffer was added into a polystyrene 384-well assay plate (BD Biosciences).

Compound solutions (8 μL/well) at various concentrations (0-300 μM) were added to the assay plate (columns 5-24). Compound solution contains 30 mM glycylglycine, pH 7.3, 40 mM KCl, 20 mM MgCl₂, 9.2% DMSO, with (columns 15-24) or without (columns 5-14) 20 mM glucose 6-phosphate.

Enzyme solution (12 μL/well) containing glycogen synthase (16.88 μg/ml), pyruvate kinase (0.27 mg/ml), lactate dehydrogenase (0.27 mg/ml) in 50 mM Tris-HCl, pH 8.0, 27 mM DTT and bovine serum albumin (BSA, 0.2 mg/ml) was added to the assay plate (columns 3-24). As a blank control, enzyme solution without glycogen synthase was added into the top half wells of columns 1-2. To the bottom half wells of columns 1-2 were added a known activator, glucose 6-phosphate (at final concentration 5 mM) in addition to the enzyme solution. The reaction mixture was incubated at room temperature. The assay plate was then read for absorbance at 340 nm on an Envision reader every 3 minutes up to a total of 15 minutes.

The enzyme activity (with or without compound) was calculated by the reaction rate and represented by the optical density change (δOD) per minute. Percent stimulation of glycogen synthase activity by a compound at various concentrations was calculated by the following formula:

% stimulation=100*Rs/Rt,

Where Rs is the reaction rate of the enzyme in the presence of compound and Rt is the reaction rate of the enzyme in the absence of compound.

SC₂₀₀ is defined as the compound concentration that is needed to stimulate 200% of the enzyme activity. EC₅₀ is defined as the compound concentration that is needed to give 50% maximum activation.

Compounds from Examples 1 through Example 64 were assayed according to assay procedures described above and the result is listed in Table 1 below:

TABLE 1
Glycogen Synthase Activation Potency of Exemplified Compounds

| Example Number | GS SC200 (μM) | GS EC50 (μM) |
|---|---|---|
| 1 | 0.21 | 1.34 |
| 2 | 0.13 | 1.08 |
| 3 | 0.11 | 0.69 |
| 4 | 0.18 | 0.72 |
| 5 | 0.09 | 0.45 |
| 6 | 0.03 | 0.47 |
| 7 | 0.04 | 0.31 |
| 8 | 0.17 | 0.91 |
| 9 | 0.06 | 0.52 |
| 10 | 0.03 | 0.29 |
| 11 | 0.97 | 3.67 |
| 12 | 0.75 | 4.31 |
| 13 | 0.10 | 0.93 |
| 14 | 0.15 | 1.31 |
| 15 | 0.15 | 1.43 |
| 16 | 0.43 | 1.54 |
| 17 | 0.36 | 1.53 |
| 18 | 0.07 | 0.16 |
| 19 | 0.42 | 1.82 |
| 20 | 0.50 | 5.24 |
| 21 | 0.16 | 0.43 |
| 22 | 0.03 | 0.26 |
| 23 | 0.10 | 0.37 |
| 24 | 0.12 | 0.73 |
| 25 | 0.07 | 0.41 |
| 26 | 0.05 | 0.45 |
| 27 | 0.08 | 0.52 |
| 28 | 0.06 | 0.41 |
| 29 | 0.01 | 0.34 |
| 30 | 0.04 | 0.47 |
| 31 | 0.07 | 0.48 |
| 32 | 0.07 | 0.61 |
| 33 | 0.02 | 0.35 |
| 34 | 0.04 | 0.28 |
| 35 | 0.46 | 1.46 |
| 36 | 0.50 | 1.93 |
| 37 | 0.38 | 4.90 |
| 38 | 0.05 | 0.13 |
| 39 | 0.03 | 0.18 |
| 40 | 0.04 | 0.20 |
| 41 | 0.07 | 0.15 |
| 42 | 0.06 | 0.18 |
| 43 | 0.03 | 0.19 |
| 44 | 0.09 | 0.38 |
| 45 | 0.02 | 0.12 |
| 46 | 0.01 | 0.09 |
| 47 | 0.62 | 1.82 |
| 48 | 0.01 | 0.11 |
| 49 | 0.35 | 0.89 |
| 50 | 0.42 | 0.84 |
| 51 | 0.03 | 0.10 |
| 52 | 0.02 | 0.12 |
| 53 | 0.03 | 0.13 |
| 54 | 0.90 | 3.71 |
| 55 | 0.16 | 0.33 |
| 56 | 0.04 | 0.11 |
| 57 | 1.22 | 2.34 |
| 58 | 0.04 | 0.06 |
| 59 | 6.06 | 16.59 |
| 60 | 1.74 | 2.19 |
| 61 | 0.78 | 1.17 |
| 62 | 0.26 | 0.43 |
| 63 | 0.01 | 0.55 |
| 64 | 0.31 | 0.55 |

It is to be understood that the invention is not limited to the particular embodiments of the invention described above, as variations of the particular embodiments may be made and still fall within the scope of the appended claims.

What is claimed is:

1. A compound of formula (I): 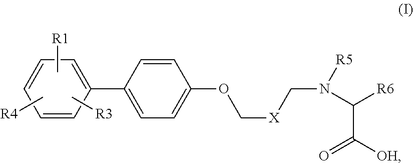
wherein:
R1, R2, R3, independently of each other, is hydrogen, halogen, lower alkyl or alkoxy;
X is pyridine, thiazole, unsubstituted phenyl or phenyl substituted with R4;
R4 is halogen;
R5 is hydrogen, an acyl moiety, —SO₂-lower alkyl, —SO₂-aryl, —SO₂-cycloalkyl, or unsubstituted lower alkyl or lower alkyl substituted with phenyl;
R6 is hydrogen or lower alkyl; or
R5 and R6, together with the nitrogen atom to which they are attached, form a 5- or 6-membered heterocyclic ring, optionally containing a further heteroatom selected from oxygen or sulfur, said heterocyclic ring being unsubstituted or mono- or bi-substituted with (═O),
or a pharmaceutically acceptable salt thereof.

2. The compound according to claim 1, wherein X is unsubstituted phenyl or phenyl substituted with R4; R5 is hydrogen, an acyl moiety, —SO₂-lower alkyl, —SO₂-aryl, —SO₂-cycloalkyl, unsubstituted lower alkyl or lower alkyl substituted with phenyl; and R6 is hydrogen.

3. The compound according to claim 1, wherein X is unsubstituted phenyl or phenyl substituted with R4; and R5 and R6, together with the nitrogen atom to which they are attached, form a 5- or 6-membered heterocyclic ring, optionally containing a further heteroatom selected from oxygen or sulfur, said heterocyclic ring being unsubstituted or mono- or bi-substituted with (═O).

4. The compound according to claim 1, wherein X is thiazole or pyridine; R5 is hydrogen, an acyl moiety, —SO₂-lower alkyl, —SO₂-aryl, —SO₂-cycloalkyl, unsubstituted lower alkyl or lower alkyl substituted with phenyl; and R6 is hydrogen.

5. The compound according to claim 1, wherein X is thiazole or pyridine; and R5 and R6, together with the nitrogen atom to which they are attached, form a 5- or 6-membered heterocyclic ring, optionally containing a further heteroatom selected from oxygen or sulfur, said heterocyclic ring being unsubstituted or mono- or bi-substituted with (═O).

6. The compound according to claim 1, wherein R1, R2, R3, independently of each other, is hydrogen, fluoro, chloro, methyl or methoxy.

7. The compound according to claim 1, wherein R1 is hydrogen or fluoro.

8. The compound according to claim 1, wherein R2 is fluoro.

9. The compound according to claim 1, wherein R3 is fluoro, chloro or methoxy.

10. The compound according to claim 1, wherein X is unsubstituted phenyl.

11. The compound according to claim 1, wherein X is thiazole.

12. The compound according to claim 1, wherein X is pyridine.

13. The compound according to claim 1, wherein R4 is fluorine.

14. The compound according to claim 1, wherein R5 is an acyl moiety.

15. The compound according to claim 1, wherein R5 is an acyl moiety selected from the group consisting of: —C(O)-lower alkyl, branched or unbranched, unsubstituted or substituted with alkoxy or cycloalkyl, —C(O)-cycloalkyl, —C(O)-heterocycloalkyl, unsubstituted or substituted with methyl, —C(O)-aryl, —C(O)-alkoxy, and —C(O)-heteroaryl, unsubstituted or substituted with methyl.

16. The compound according to claim 1, wherein R5 is an acyl moiety selected from the group consisting of: —C(O)C(CH₃)₃, —C(O)CH₂CH(CH₃)₂, —C(O)-morpholine, —C(O)-cyclobutane, —C(O)-phenyl, —C(O)OCH(CH₃)₂, —C(O)-methylimidazole, —C(O)-pyridine, —C(O)C(CH₃)CH₂OCH₃, —C(O)OCH₃, —C(O)OCH₂CH₃, —C(O)CH₃, —C(O)-cyclopropane, —C(O)CH₂CH₃, —C(O)CH₂-cyclopropane, —C(O)-tetrahydrofuran, —C(O)CH₂CH₂OCH₃, —C(O)-thiazole, —C(O)CH₂OCH₃ and —C(O)-methylpiperidine.

17. The compound according to claim 1, wherein R5 is hydrogen, —SO₂-lower alkyl, —SO₂-aryl, —SO₂-cycloalkyl, unsubstituted lower alkyl or lower alkyl substituted with phenyl.

18. The compound according to claim 1, wherein R5 is hydrogen, —SO₂CH₂CH₃, —SO₂-phenyl, —SO₂-cyclopentane, —SO₂CH₃, —CH₂-phenyl or —CH₂CH₃.

19. The compound according to claim 1, wherein R5 and R6, together with the nitrogen atom to which they are attached, form a 5- or 6-membered heterocyclic ring, optionally containing a further heteroatom selected from oxygen or sulfur, said heterocyclic ring being unsubstituted or mono- or bi-substituted with (═O).

20. The compound according to claim 1, wherein said 5- or 6-membered heterocyclic ring is dioxo-isothiazolidine, oxo-pyrrolidine, pyrrolidine or dioxo-thiazinane.

21. The compound according to claim 1, wherein R6 is hydrogen.

22. The compound according to claim 1, wherein said compound is:
[[3-(4′,5′-Difluoro-2′-methoxy-biphenyl-4-yloxymethyl)-benzyl]-(2,2-dimethyl-propionyl)-amino]-acetic acid;
[[3-(4′,5′-Difluoro-2′-methoxy-biphenyl-4-yloxymethyl)-benzyl]-(3-methyl-butyryl)-amino]-acetic acid;
[[3-(4′,5′-Difluoro-2′-methoxy-biphenyl-4-yloxymethyl)-benzyl]-(morpholine-4-carbonyl)-amino]-acetic acid;
{Cyclobutanecarbonyl-[3-(4′,5′-difluoro-2′-methoxy-biphenyl-4-yloxymethyl)-benzyl]-amino}-acetic acid;
{Benzoyl-[3-(4′,5′-difluoro-2′-methoxy-biphenyl-4-yloxymethyl)-benzyl]-amino}-acetic acid;
{[3-(4′,5′-Difluoro-2′-methoxy-biphenyl-4-yloxymethyl)-benzyl]-isopropoxycarbonyl-amino}-acetic acid;
{[3-(4′,5′-Difluoro-2′-methoxy-biphenyl-4-yloxymethyl)-benzyl]-ethanesulfonyl-amino}-acetic acid;
{Benzenesulfonyl-[3-(4′,5′-difluoro-2′-methoxy-biphenyl-4-yloxymethyl)-benzyl]-amino}-acetic acid;
{Cyclopropanesulfonyl-[3-(4′,5′-difluoro-2′-methoxy-biphenyl-4-yloxymethyl)-benzyl]-amino}-acetic acid;
{[3-(4′,5′-Difluoro-2′-methoxy-biphenyl-4-yloxymethyl)-benzyl]-methanesulfonyl-amino}-acetic acid;
[[3-(2′,4′-Difluoro-biphenyl-4-yloxymethyl)-benzyl]-(2,2-dimethyl-propionyl)-amino]-acetic acid;
{Cyclopropanecarbonyl-[3-(2′,4′-difluoro-biphenyl-4-yloxymethyl)-benzyl]-amino}-acetic acid;
[[3-(2′,4′-Difluoro-biphenyl-4-yloxymethyl)-benzyl]-(morpholine-4-carbonyl)-amino]-acetic acid;
[[3-(2′,4′-Difluoro-biphenyl-4-yloxymethyl)-benzyl]-(1-methyl-imidazole-2-carbonyl)-amino]-acetic acid;
[[3-(2′,4′-Difluoro-biphenyl-4-yloxymethyl)-benzyl]-(pyridine-3-carbonyl)-amino]-acetic acid;
[[3-(2′,4′-Difluoro-biphenyl-4-yloxymethyl)-benzyl]-(pyridine-2-carbonyl)-amino]-acetic acid;
[[3-(2′,4′-Difluoro-biphenyl-4-yloxymethyl)-benzyl]-(3-methoxy-2-methyl-propionyl)-amino]-acetic acid;
{Ethanesulfonyl-[3-(2′,4′,5′-trifluoro-biphenyl-4-yloxymethyl)-benzyl]-amino}-acetic acid;
{[3-(4′,5′-Difluoro-2′-methoxy-biphenyl-4-yloxymethyl)-benzyl]-methoxycarbonyl-amino}-acetic acid; or
{[3-(4′,5′-Difluoro-2′-methoxy-biphenyl-4-yloxymethyl)-benzyl]-ethoxycarbonyl-amino}-acetic acid.

23. The compound according to claim 1, wherein said compound is:
{Acetyl-[3-(4′,5′-difluoro-2′-methoxy-biphenyl-4-yloxymethyl)-benzyl]-amino}-acetic acid;
{Cyclopropanecarbonyl-[3-(4′,5′-difluoro-2′-methoxy-biphenyl-4-yloxymethyl)-benzyl]-amino}-acetic acid;
{[3-(4′,5′-Difluoro-2′-methoxy-biphenyl-4-yloxymethyl)-benzyl]-propionyl-amino}-acetic acid;
{(2-Cyclopropyl-acetyl)-[3-(4′,5′-difluoro-2′-methoxy-biphenyl-4-yloxymethyl)-benzyl]-amino}-acetic acid;
[[3-(4′,5′-Difluoro-2′-methoxy-biphenyl-4-yloxymethyl)-benzyl]-((S)-tetrahydro-furan-2-carbonyl)-amino]-acetic acid;
[[3-(4′,5′-Difluoro-2′-methoxy-biphenyl-4-yloxymethyl)-benzyl]-(3-methoxy-propionyl)-amino]-acetic acid;
[[3-(4′,5′-Difluoro-2′-methoxy-biphenyl-4-yloxymethyl)-benzyl]-(thiazole-4-carbonyl)-amino]-acetic acid;
[[3-(4′,5′-Difluoro-2′-methoxy-biphenyl-4-yloxymethyl)-benzyl]-(2-methoxy-acetyl)-amino]-acetic acid;
[[3-(4′,5′-Difluoro-2′-methoxy-biphenyl-4-yloxymethyl)-benzyl]-(1-methyl-1H-imidazole-4-carbonyl)-amino]-acetic acid;
[[3-(4′,5′-difluoro-2′-methoxy-biphenyl-4-yloxymethyl)-benzyl]-(pyridine-3-carbonyl)-amino]-acetic acid;
[3-(4′,5′-Difluoro-2′-methoxy-biphenyl-4-yloxymethyl)-benzyl]-(3-methoxy-2-methyl-propionyl)-amino]-acetic acid;
[[3-(4′,5′-Difluoro-2′-methoxy-biphenyl-4-yloxymethyl)-benzyl]-(1-methyl-piperidine-4-carbonyl)-amino]-acetic acid;
[[3-(4′,5′-Difluoro-2′-methoxy-biphenyl-4-yloxymethyl)-benzyl]-((R)-tetrahydro-furan-2-carbonyl)-amino]-acetic acid;
[[3-(4′,5′-Difluoro-2′-methoxy-biphenyl-4-yloxymethyl)-benzyl]-(tetrahydro-furan-3-carbonyl)-amino]-acetic acid;
{Benzyl-[3-(4′,5′-difluoro-2′-methoxy-biphenyl-4-yloxymethyl)-benzyl]-amino}-acetic acid;
{[3-(4′,5′-Difluoro-2′-methoxy-biphenyl-4-yloxymethyl)-benzyl]-ethyl-amino}-acetic acid;
[3-(4′,5′-Difluoro-2′-methoxy-biphenyl-4-yloxymethyl)-benzylamino]-acetic acid;
1,1-Dioxo-2-[3-(2′,4′,5′-trifluoro-biphenyl-4-yloxymethyl)-benzyl]-1lambda*6*-isothiazolidine-3-carboxylic acid;
2-[3-(4′,5′-Difluoro-2′-methyl-biphenyl-4-yloxymethyl)-benzyl]-1,1-dioxo-1lambda*6*-isothiazolidine-3-carboxylic acid; or
2-[3-(2′-Chloro-4′,5′-difluoro-biphenyl-4-yloxymethyl)-benzyl]-1,1-dioxo-1 lambda*6*-isothiazolidine-3-carboxylic acid.

24. The compound according to claim 1, wherein said compound is:
2-[3-(2′,4′-Difluoro-biphenyl-4-yloxymethyl)-4-fluoro-benzyl]-1,1-dioxo-1lambda*6*-isothiazolidine-3-carboxylic acid;
2-[5-(2′,4′-Difluoro-biphenyl-4-yloxymethyl)-2-fluoro-benzyl]-1,1-dioxo-1lambda*6*-isothiazolidine-3-carboxylic acid;
2-[3-(4′,5′-Difluoro-2′-methoxy-biphenyl-4-yloxymethyl)-benzyl]-1,1-dioxo-1lambda*6*-isothiazolidine-3-carboxylic acid;
(R)-2-[3-(4′,5′-Difluoro-2′-methoxy-biphenyl-4-yloxymethyl)-benzyl]-1,1-dioxo-1lambda*6*-isothiazolidine-3-carboxylic acid;
(S)-2-[3-(4′,5′-Difluoro-2′-methoxy-biphenyl-4-yloxymethyl)-benzyl]-1,1-dioxo-1lambda*6*-isothiazolidine-3-carboxylic acid;
(S)-1,1-Dioxo-2-[3-(2′,4′,5′-trifluoro-biphenyl-4-yloxymethyl)-benzyl]-1lambda*6*-isothiazolidine-3-carboxylic acid;
(R)-1,1-Dioxo-2-[3-(2′,4′,5′-trifluoro-biphenyl-4-yloxymethyl)-benzyl]-1lambda*6*-isothiazolidine-3-carboxylic acid;
(S)-2-[3-(4′,5′-Difluoro-2′-methyl-biphenyl-4-yloxymethyl)-benzyl]-1,1-dioxo-1lambda*6*-isothiazolidine-3-carboxylic acid;
(R)-2-[3-(4′,5′-Difluoro-2′-methyl-biphenyl-4-yloxymethyl)-benzyl]-1,1-dioxo-1lambda*6*-isothiazolidine-3-carboxylic acid;
(R)-2-[3-(2′-Chloro-4′,5′-difluoro-biphenyl-4-yloxymethyl)-benzyl]-1,1-dioxo-1lambda*6*-isothiazolidine-3-carboxylic acid;
(S)-2-[3-(2′-Chloro-4′,5′-difluoro-biphenyl-4-yloxymethyl)-benzyl]-1,1-dioxo-1lambda*6*-isothiazolidine-3-carboxylic acid;
(S)-1-[3-(4′,5′-Difluoro-2′-methoxy-biphenyl-4-yloxymethyl)-benzyl]-5-oxo-pyrrolidine-2-carboxylic acid;
(S)-5-Oxo-1-[3-(2′,4′,5′-trifluoro-biphenyl-4-yloxymethyl)-benzyl]-pyrrolidine-2-carboxylic acid;
(S)-1-[3-(2′,4′-Difluoro-biphenyl-4-yloxymethyl)-benzyl]-pyrrolidine-2-carboxylic acid trifluoro-acetic acid salt;
(R)-2-[3-(4′,5′-Difluoro-2′-methoxy-biphenyl-4-yloxymethyl)-benzyl]-1,1-dioxo-1lambda*6*-[1,2]thiazinane-3-carboxylic acid;
(S)-2-[3-(4′,5′-Difluoro-2′-methoxy-biphenyl-4-yloxymethyl)-benzyl]-1,1-dioxo-1lambda*6*-[1,2]thiazinane-3-carboxylic acid;
(R)-1,1-Dioxo-2-[3-(2′,4′,5′-trifluoro-biphenyl-4-yloxymethyl)-benzyl]-1lambda*6*-[1,2]thiazinane-3-carboxylic acid;
(S)-1,1-Dioxo-2-[3-(2′,4′,5′-trifluoro-biphenyl-4-yloxymethyl)-benzyl]-1lambda*6*-[1,2]thiazinane-3-carboxylic acid;
(R)-1,1-Dioxo-2-[2-(2′,4′,5′-trifluoro-biphenyl-4-yloxymethyl)-thiazol-4-ylmethyl]-1lambda*6*-isothiazolidine-3-carboxylic acid;
(S)-1,1-Dioxo-2-[2-(2′,4′,5′-trifluoro-biphenyl-4-yloxymethyl)-thiazol-4-ylmethyl]-1lambda*6*-isothiazolidine-3-carboxylic acid;
(R)-2-[2-(4′,5′-Difluoro-2′-methoxy-biphenyl-4-yloxymethyl)-thiazol-4-ylmethyl]-1,1-dioxo-1lambda*6*-isothiazolidine-3-carboxylic acid;
(S)-2-[2-(4′,5′-Difluoro-2′-methoxy-biphenyl-4-yloxymethyl)-thiazol-4-ylmethyl]-1,1-dioxo-1lambda*6*-isothiazolidine-3-carboxylic acid;
2-[5-(4′,5′-Difluoro-2′-methoxy-biphenyl-4-yloxymethyl)-pyridin-3-ylmethyl]-1,1-dioxo-1lambda*6*-isothiazolidine-3-carboxylic acid; or
2-[5-(4′,5′-Difluoro-2′-methoxy-biphenyl-4-yloxymethyl)-pyridin-3-ylmethyl]-1,1-dioxo-1lambda*6*-isothiazolidine-3-carboxylic acid.

25. A pharmaceutical composition, comprising a therapeutically effective amount of a compound according to claim 1 and a pharmaceutically acceptable carrier and/or adjuvant.
